(19) United States
(12) Patent Application Publication
(10) Pub. No.: US 2014/0288541 A1
(43) Pub. Date: Sep. 25, 2014

(54) LESION TREATMENT DEVICE AND METHODS FOR TREATING LESIONS

(71) Applicant: ASYMMETRIC MEDICAL LTD., Shdema (IL)

(72) Inventors: Moshe ESHKOL, Harutzim (IL); Ori WEISBERG, Shdema (IL)

(73) Assignee: ASYMMETRIC MEDICAL LTD., Shdema (IL)

(21) Appl. No.: 14/293,058

(22) Filed: Jun. 2, 2014

Related U.S. Application Data
(63) Continuation-in-part of application No. PCT/IL2012/050496, filed on Dec. 4, 2012.
Application No. PCT/IL2012/050496 is a continuation-in-part of application No. PCT/IL2013/050099, filed on Jan. 31, 2013.
(60) Provisional application No. 61/566,667, filed on Dec. 4, 2011.
(60) Provisional application No. 61/592,602, filed on Jan. 31, 2012.
(60) Provisional application No. 61/847,090, filed on Jul. 17, 2013.

(51) Int. Cl. A61B 18/24; G06F 17/50

(52) U.S. Cl. CPC A61B 18/245; G06F 17/50; A61B 2018/00422
USPC 606/7; 703/7

(57) ABSTRACT

Tissue and vessel treatment devices and respective methods are provided. Devices comprise a flexible treatment layer that comprises a plurality of operable elements configured to be activated to apply the treatment to the obstruction upon bending of the flexible treatment layer beyond a specified curvature threshold and to be de-activated upon a specified decrease of the bending. The treatment layer may comprise optical fiber(s) with the operable elements as emission regions that emit electromagnetic radiation from the core upon bending the optical fiber beyond a specified bending threshold. Devices may be configured as vessel sealing tips for surgical forceps, in which the treatment layer is configured to yield vessel welding and vessel cutting effects.

### CROSS REFERENCE TO RELATED APPLICATIONS

This application is a continuation-in-part of International Patent Application No. PCT/IL2012/050496, filed on Dec. 4, 2012, which claims the benefit of U.S. Provisional Patent Application No. 61/566,667, filed Dec. 4, 2011. This application also claims priority as a continuation-in-part to International Patent Application No. PCT/IL2013/050099, filed on Jan. 31, 2013, which claims priority from U.S. Provisional Patent Application No. 61/592,602, filed Jan. 31, 2012. This application also claims the benefit of U.S. Provisional Patent Application No. 61/847,090, filed Jul. 17, 2013. Each such noted application is herein incorporated by reference in its entirety.

### BACKGROUND OF THE INVENTION

1. Technical Field

The present invention relates to the field of treating flow obstructions or protrusions in vessels and/or passages in biological systems, and more particularly, to cardiovascular lesion treatment.

2. Discussion of Related Art

Atherosclerosis, which is a major cause of cardiovascular disease resulting in myocardial infarction, stroke and other major medical complications, is characterized by the progressive accumulation of atherosclerotic deposits (known as plaque) on the inner walls of arteries. Consequently, blood flow is obstructed and there is increased likelihood of clot formation that can partially or completely block or occlude an artery. Even in cases where occlusion doesn't occur, plaque is a risk factor as certain non-stable deposits, known as vulnerable plaque, may dislodge and cause a stroke or myocardial infarction. Arteries narrowed as a result of atherosclerosis that cannot be treated effectively by drug therapy are treated by medical procedures designed to restore blood flow, including highly invasive procedures such as coronary artery bypass surgery and less invasive procedures such as balloon angioplasty, atherectomy and stenting.

Bypass surgery involves opening the patient's chest and transferring a vein cut from the patient's leg to the heart to construct a detour around the occluded artery. Bypass surgery requires prolonged hospitalization and an extensive recuperation period. Furthermore, bypass surgery also exposes the patient to a risk of major surgical complications.

Balloon angioplasty is a less invasive and costly alternative to bypass surgery and is performed in a hospital cardiac catheterization laboratory by an interventional cardiologist. In this procedure, a balloon-tipped catheter is inserted into a blood vessel through a small incision in the patient's arm or leg. The physician uses a guide catheter to feed the balloon through the patient's blood vessels to the occluded artery. At that point, a guidewire is inserted across the deposits of atherosclerotic plaque, known as lesions, to provide a pathway for the balloon catheter. The deflated balloon is advanced over the guidewire, positioned within the occluded area and inflated and deflated several times. This inflation and deflation usually tears the plaque and expands the artery beyond its point of elastic recoil. Thus, although no plaque is removed the opening through which the blood flows is enlarged.

Atherectomy employs a rotating mechanical device mounted on a catheter to cut and remove plaque from a diseased artery.

Another approach to treating atherosclerosis or thrombosis is to degrade thrombi and remove plaque using various pharmacologic agents. Many techniques currently exist to deliver medicaments and other active agents to body tissues. These include oral administration, direct injection to the tissue and intravenous administration. These mechanisms are systemic in that they deliver the active agent via the bloodstream throughout the entire body. Effective pharmacologic or drug therapy requires achieving adequate concentrations of the active drug at the desired treatment site without producing drug concentrations elsewhere in the body that may cause undesirable and even dangerous side effects.

Laser angioplasty removes plaques using light in varying wavelengths ranging from ultraviolet to infrared that is delivered to the lesion by a fiber-optic catheter. Early attempts to develop a laser angioplasty system used continuous wave thermal lasers that generated heat to vaporize plaque. These laser systems caused charring and significant thermal damage to healthy tissue surrounding the lesion. As a result, thermal laser systems have generally been regarded as inappropriate for use in coronary arteries. Conversely, excimer lasers use ultraviolet light to break the molecular bonds of the atherosclerotic plaque, a process known as photoablation. Excimer lasers use electrically excited xenon and chloride gases to generate an ultraviolet laser pulse with a wavelength of 308 nm. This UV light wavelength is absorbed by the proteins and lipids that comprise plaque, resulting in precise plaque disintegration and thus, blood flow restoration without significant thermal damage to surrounding tissue. The ablated plaque is converted into carbon dioxide and other gases, as well and minute particulate matter that can be easily eliminated. Similarly, Ultrasound and RF are sometimes used for the ablation of plaque.

The challenge remains to detect and treat vascular lesions in an effective and safe manner.

Vessel manipulation is a commonly encountered challenge, especially in minimally invasive procedures. The variety of encountered vessels and the need to manipulate vessels without causing additional damage and bleeding require time and skill which may challenge procedure success and place a significant obstacle to the further development of such procedures.

Optical fibers are a common way of delivering electromagnetic radiation, e.g. laser light, to a target. Optical fibers deliver the radiation in a tight beam originating from the end face directly or projected sideways by a mirror. An optical fiber assembly generally consists of four parts: core, cladding, coating and jacket. The core is the region in which light is guided; it is usually covered by a lower index cladding, in the case of a Total Internal Reflection (TIR) fiber. In case of a Photonic Crystal Fiber (PCF), e.g. having a beehive-like structure, an OmniGuide having photonic bandgap mirrors, a Bragg fiber etc., the cladding actually consists of a complex structure of higher index of refraction materials (disclosed e.g., in U.S. Pat. No. 7,142,756 which is incorporated herein by reference in its entirety). In a metallic waveguide, the core is lower index and the cladding is a coated metal, usually Ag coated by silver-iodide AgI. In all these fiber types light is confined to the core region under normal conditions, while in extreme bends light escapes the core and can even escape the entire fiber.

It is well known that waveguides and fibers suffer losses in bends. These losses are caused by the rays' higher angle of incidence on the core/clad (mirror) interface. This is often a major limitation most manufacturers try to avoid; see for example the Corning ClearCurve Fiber (disclosed e.g., in U.S. Pat. No. 5,278,931 which is incorporated herein by reference in its entirety). Another approach is to use outside cabling, as in U.S. Pat. No. 4,078,853, which is incorporated herein by reference in its entirety, to limit fiber bending and thus loss. Other devices take advantage of the optical power loss in the construction of sensors based on the loss in bends (disclosed e.g., in U.S. Pat. No. 4,770,047 which is incorporated herein by reference in its entirety).

In another example, U.S. Pat. No. 5,138,676 which is incorporated herein by reference in its entirety, discloses tight bending with low loss that is achieved by reducing fiber outer diameter (OD) and confining light with respect to the lower-index surrounding. OD reduction is performed by either removing part of the cladding or drawing down the fiber in the bent section. Other aspects are disclosed by U.S. Pat. No. 5,278,931, U.S. Pat. No. 4,078,853, U.S. Pat. No. 4,770,047 and U.S. Pat. No. 5,138,676, which are incorporated herein by reference in their entirety.

Tissue cutting and treatment are performed in three major methods: scalpels (cold steel techniques), electro- and laser-surgery. The main use of laser surgery is in ablation or coagulation of soft tissue. In laser surgery, an optical fiber is often used to deliver the laser energy to the desired site with minimal losses on the way.

In the case of soft tissue cutting and most other medical uses of fibers, laser light is emitted from the tip of the fiber at the treatment site. The fiber is used to safely deliver the energy to the exact location in spot form as in VersaPulse by Lumenis. In some cases local redirection of the fiber tip is used for improved targeting as disclosed e.g., in U.S. Pat. No. 7,238,180, which is incorporated herein by reference in its entirety.

In other cases, the fiber is manipulated to allow laser emission at a 90° angle to the fiber tip, term “side firing”, which makes it easier to use in cases where it is geometrically harder to bend the fiber tip in the required direction. The straight angle is achieved using a mirror or a side fire mechanism. Sometimes when the side fire approach is used, specific treatment of the fiber edge is required in order to prevent local damage and facilitate effective tip side firing as disclosed e.g., in U.S. Patent Publication No. 20070106286, which is incorporated herein by reference in its entirety. In some cases tip side fire is enabled by tapering the fiber core as disclosed e.g., in U.S. Patent Publication No. 20110002584, which is incorporated herein by reference in its entirety. Alternatively, side firing is achieved by increasing the index of refraction of the cladding on one side by preferential heating, using an external laser source (disclosed e.g., in U.S. Pat. No. 6,606,431, which is incorporated herein by reference in its entirety).

The following US patents teach devices that utilize laser radiation for cutting. These patents are incorporates herein by reference in their entirety. U.S. Pat. No. 4,266,547 teaches a laser knife acting linearly between an emitter and an acceptor; U.S. Pat. No. 5,151,097 teaches a laser emitter that emits radiation through an uncovered area in an element that is otherwise covered by a light impervious material; U.S. Pat. No. 5,470,331 teaches a laser light transmissive probe system that is provided with an opposed pair of laser light transmissive probes for pinching tissue; U.S. Pat. No. 6,283,962 teaches a medical ablation device and U.S. Pat. No. 6,221,069 teaches an apparatus having an outer probe having a form of hook and an inner probe for contacting the outer probe and heating a target tissue disposed therebetween. U.S. Pat. Nos. 5,531,741 and 6,409,719, which are incorporates herein by reference in their entirety, teach devices that utilize an optical fiber to illuminate a scene in which stents are implants.

### SUMMARY OF THE INVENTION

One aspect of the present invention provides a device for applying a treatment to an obstruction, comprising a flexible treatment layer that comprises a plurality of operable elements configured to be activated to apply the treatment to the obstruction upon bending of the flexible treatment layer beyond a specified curvature threshold and to be de-activated upon a specified decrease of the bending, resulting from the treatment of the obstruction.

One aspect of the present invention provides a flexible treatment layer having one or more optical fibers. The fibers comprise a core and a cladding, and have at least one specified region of the cladding that is arranged to emit electromagnetic radiation from the core upon bending the optical fiber at the at least one specified region beyond a specified bending threshold. The emission regions are operable elements of the treatment layer configured to treat the obstruction.

One aspect of the present invention provides a device such as a vessel sealing tip for surgical forceps, the vessel sealing tip comprising at least one energy delivering element arranged to deliver, upon actuation, energy to a vessel to yield a vessel welding effect in a specified sealing section of the vessel and to cut the vessel within the specified sealing section, the energy delivery being carried out via the operable elements.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A-1C are high level schematic illustrations of a cross section of a vessel with an obstruction, contacted by a device for applying a treatment to the obstruction, according to some embodiments of the invention;

FIGS. 2A-2D are high level schematic illustrations of a partial longitudinal section of a vessel with a lesion, contacted by a device for applying a treatment to an obstruction such as a vascular lesion, according to some embodiments of the invention;

FIGS. 3A and 3B are high level schematic illustrations of a device configuration having a non-active state and a bending-activated state, respectively, according to some embodiments of the invention;

FIGS. 4A and 4B are high level schematic illustrations of a device for perforating the lesion, having a non-active state and a bending-activated state, respectively, according to some embodiments of the invention;

FIGS. 5A and 5B are high level schematic illustrations of a device for perforating the lesion, having a non-active state and a bending-activated state, respectively, and including an actuator, according to some embodiments of the invention;

FIGS. 6A and 6B are high level schematic illustrations of a device for delivering a drug to the lesion, having a non-active state and a bending-activated state, respectively, according to some embodiments of the invention;

FIGS. 7A and 7B are high level schematic illustrations of a device for delivering a drug to the lesion, having a non-active state and a bending-activated state, respectively, according to some embodiments of the invention;

FIGS. 8A and 8B are high level schematic illustrations of a device having a flexible network with varying mesh openings, according to some embodiments of the invention;

FIGS. 9A and 9B are high level schematic illustrations of a device for delivering a drug to the lesion, according to some embodiments of the invention;

FIGS. 10A and 10B are high level schematic illustrations of an obstruction grinding device, according to some embodiments of the invention;

FIGS. 11A-11E are high level schematic illustrations of a one layered treatment device, according to some embodiments of the invention;

FIGS. 12A-12C are high level schematic illustrations of openings as operable elements, according to some embodiments of the invention;

FIG. 13A is a high level schematic illustration of a balloon implemented device for treating lesions at vessel bifurcations, according to some embodiments of the invention;

FIG. 13B is a high level schematic illustration of a device for treating flow obstructions by a supportive structure, according to some embodiments of the invention.

FIGS. 13C-13K are high level schematic illustrations of a device having optical fibers exhibiting bend emission wound on a supportive structure such as a balloon, according to some embodiments of the invention;

FIGS. 14A-14C are high level schematic illustrations of a parachute implemented device in stages of operation, according to some embodiments of the invention;

FIGS. 15A and 15B are high level schematic illustrations of a device for focusing energy at the lesion, having a non-active state and a bending-activated state, respectively, according to some embodiments of the invention; FIGS. 15C and 15D demonstrate bend emission for the fiber configuration illustrated in FIG. 1711 (see below, serrated fiber), according to some embodiments of the invention;

FIG. 16A is a high level schematic block diagram of an emitting region of an optical fiber, according to some embodiments of the invention;

FIG. 16B is a schematic illustration of the dependency of the emitted radiation through the specified region on the bending radius, according to some embodiments of the invention;

FIG. 16C is a schematic illustration of the dependency of the emitted radiation through the specified region on the bending radius (y axis) and on the ratio n_{E}/n_{K} (x axis), according to some embodiments of the invention;

FIGS. 17A-17I and 17L are high level schematic illustrations of various embodiments of the operable elements of the optical fiber having an asymmetric cladding in cross section, according to some embodiments of the invention;

FIG. 17J illustrates an embodiment of the fiber with a coating that has a gap or is missing at the specified emission region, according to some embodiments of the invention;

FIG. 17K illustrates an embodiment of a metallic waveguide having a gap at the specified emission region, according to some embodiments of the invention;

FIG. 18A is a high level schematic illustration of the fiber with a flat supportive structure attached on an opposite side of the fiber with respect to the emitting region and the target, according to some embodiments of the invention

FIG. 18B is a high level schematic illustration of the fiber with lateral circle segments, according to some embodiments of the invention;

FIG. 18C is a high level schematic illustration of the fiber with a circular segment at the fiber tip, according to some embodiments of the invention;

FIGS. 19A-19C, 19E, 19F, 19H and 19I are high level schematic illustrations of devices using bend emission, according to some embodiments of the invention;

FIG. 19D illustrates an experimental comparison between a fiber snare device according to some embodiments of the invention and an electrosurgical snare regarding the emission profile;

FIGS. 20A-20C illustrate configurations of controlling the electromagnetic radiation transmitted through the fiber, according to some embodiments of the invention;

FIG. 21A-21E illustrate the cladding of the fiber, becoming asymmetric in cross section upon application of a strain at the specified region, according to some embodiments of the invention;

FIGS. 22A-22C schematically illustrate operable elements on a fiber having three or more emission sectors in the emission region and a hook device using this fiber, according to some embodiments of the invention;

FIGS. 23A-23F schematically illustrate a device configured as tweezers, according to some embodiments of the invention; Note 23F

FIGS. 24A and 24B schematically illustrate a device with a feedback loop for controlling emitted radiation, according to some embodiments of the invention;

FIG. 25 schematically illustrates a device for cutting vessels, according to some embodiments of the invention; and

FIGS. 26A and 26B illustrate a device for treating target by pressing device upon it, according to some embodiments of the invention.

FIGS. 27A-27C are high level schematic illustrations of a vessel sealing tip for surgical forceps according to some embodiments of the invention;

FIGS. 28A-28C are high level schematic illustrations of a vessel sealing tip for surgical forceps having focusing elements, according to some embodiments of the invention;

FIGS. 29A and 29B are high level schematic illustrations of a vessel sealing tip for surgical forceps having vessel piercing elements, according to some embodiments of the invention;

FIGS. 30A and 30B are high level schematic illustrations of a vessel sealing tip for surgical forceps having transversely expanding elements, according to some embodiments of the invention;

FIG. 31 is a high level schematic illustration of a vessel sealing tip for surgical forceps enabling extension of the vessel sealing region, according to some embodiments of the invention;

FIG. 32 is a high level schematic illustration of a vessel sealing tip for surgical forceps with variable intensity treatment, according to some embodiments of the invention; and

FIG. 33 is a high level flowchart illustrating a method, according to some embodiments of the invention.

### DETAILED DESCRIPTION OF THE INVENTION

Prior to setting forth the detailed description, it may be helpful to set forth definitions of certain terms that will be used hereinafter.

The term “obstruction” as used herein in this application refers to any piece of matter that retards flow through a vessel. In particular, the term “obstruction” as used herein in this application includes lesions, or vascular lesions, which are deposits on arteries (e.g., plaque) as well as other distractions to normal blood flow. The terms “obstruction” and “lesion” are used interchangeably throughout the specifications, in a non-limiting manner.

The term “vessel” as used herein in this application refers to any hollow container or pipe for moving fluids. For example, the term “vessel” refers to a blood vessel, especially an artery, parts of the gastrointestinal tract, fallopian tubes, urinary or bile tracts, airways and bronchi and other bodily vessels, as well as to pipes and tubes for industrial uses. The term “vessel” is used in this application in two contexts, first in referring to vessels that enclose treated tissue such as an obstruction (e.g., a lesion in a blood vessel) and second in referring to the vessel as the treated tissue itself (e.g., welding or cutting vessels). The term “vessel” is denoted by numerals 90 or 95, depending on the context.

The terms “energy” or “treatment energy” as used herein in this application refer to any type of energy which is usable for treating or affecting vessels, for example mechanical energy, electromagnetic energy in any form (e.g., optical energy, laser energy in any effective bandwidth, radiofrequency radiation—RF etc.), electrical or magnetic energy (e.g., electric currents or magnetic fields), heat, ultrasonic radiation etc.

The terms “treatment layer” and “supporting layer” as used herein in this application refer to device elements which are involved, respectively, in energy emission and in structural support. The term “operable elements” as used herein in this application refers to any element that is associated with the treatment layer and delivers energy of any type. The term “operable elements” is further used herein in this application in a broader sense to refer to sensing and material delivery functions of the operable elements, in addition to energy delivery. The association between the operable elements and the treatment layer may be of different kinds. Non-limiting examples are mechanical structures (e.g., a surface with pores, a flexible sheet with orienting structures), radiation emitting structures (e.g., optical fibers which emit by bending), and combinations thereof (e.g., structures which direct bending and bend-emitted energy). The supporting layer may be present (e.g., a balloon, forceps jaws, a stent etc.) or be embodied into the treatment layer (e.g., balloon skin or stent surface as the treatment layer, optical fiber with emitting regions). Specifically, the terms “bend-emitting region”, “emitting region” etc. of a fiber, as used herein in this application refer to is a region of the fiber which is implemented as an operable element, according to the principles specified below.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

Tissue and vessel treatment devices and respective methods are provided. Devices comprise a flexible treatment layer that comprises a plurality of operable elements configured to be activated to apply the treatment to the obstruction upon bending of the flexible treatment layer beyond a specified curvature threshold and to be de-activated upon a specified decrease of the bending. The treatment layer may comprise optical fiber(s) with the operable elements as emission regions that emit electromagnetic radiation from the core upon bending the optical fiber beyond a specified bending threshold. Devices may be configured as vessel sealing tips for surgical forceps, in which the treatment layer is configured to yield vessel welding and vessel cutting effects.

The present invention enables removing and/or treating obstructions in vessels and in some cases simultaneously delivering pharmacologic therapy to a selected site in the body lumen in a simple way. The invention may treat any type of obstruction in any type of vessel, combines an automatic mechanical obstruction identification mechanism with a treatment procedure to remove and/or treat the obstruction. In an example of the obstruction being a vascular lesion, the invention may be used to remove plaque without generating embolic material which can create a risk of ischemic stroke. Furthermore, the devices described by this invention automatically identify and restrict treatment to the sclerotic area, leaving adjacent tissue unharmed.

Among other, this invention relates to methods and apparatuses for excising and/or treating mobile and non-mobile atheromas. The device includes an automated lesion identification mechanism and a treatment mechanism. In general both automated lesion detection unit and the treatment head may be inserted into the body via a catheter which may operate in different regions of the human body. For example, the aorta, common carotid artery, external and internal carotid arteries, brachiocephalic trunk, middle cerebral artery, anterior cerebral artery, posterior cerebral artery, vertebral artery, basilar artery, subclavian artery, brachial artery, axillary artery, Iliac artery, renal artery, femoral artery, popliteal artery, celiac artery, superior mesenteric artery, inferior mesenteric artery, anterior tibial artery, posterior tibial artery, the coronary arteries and all other arteries. The catheter may optionally include a blood filter means which enable to capture plaque inadvertently dislodged during an atherectomy procedure.

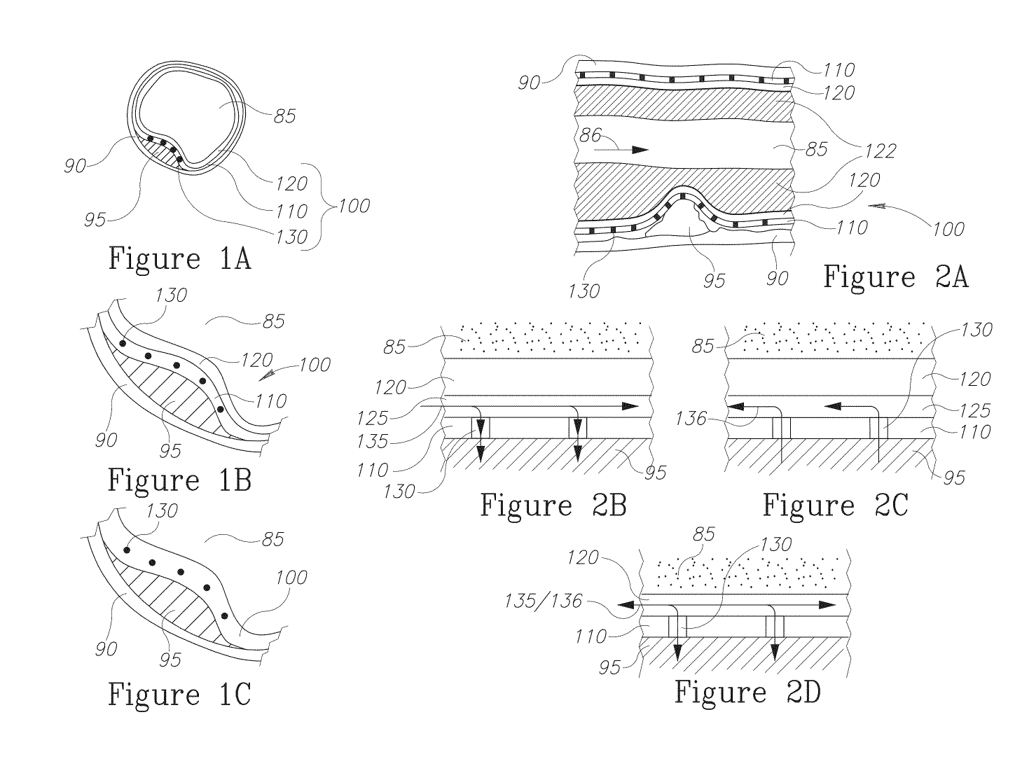

FIGS. 1A-1C are high level schematic illustrations of a cross section of a vessel 90 with an obstruction 95, contacted by a device 100 for applying a treatment to obstruction 95, according to some embodiments of the invention. FIGS. 2A-2D are high level schematic illustrations of a partial longitudinal section of vessel 90 contacted by device 100 for applying a treatment to obstruction 95 such as a vascular lesion, according to some embodiments of the invention.

Device 100 comprises a flexible supporting layer 120 and a treatment layer 110 comprising a plurality of operable elements 130 attached to flexible supporting layer 120 and configured to be mechanically activated to apply the treatment to obstruction 95 upon bending of flexible supporting layer 120 due to its contact with obstruction 95. For example, operable elements 130 may be mechanically activated upon bending of flexible supporting layer 120 beyond a specified curvature threshold, as illustrated in examples below. Generally, a smaller curvature radius causes a stronger bending which activated device 100. Upon reduction if the curvature radius, de-activation may commence when the curvature radius is larger than the threshold. Hence the expression “beyond a specified curvature threshold” in the application should be interpreted as relating to a larger curvature and a smaller curvature radius.

The bending of device 100 may result from its contact with obstruction 95 or by active pressing of device 100 upon obstruction 95 to activate operable elements 130.

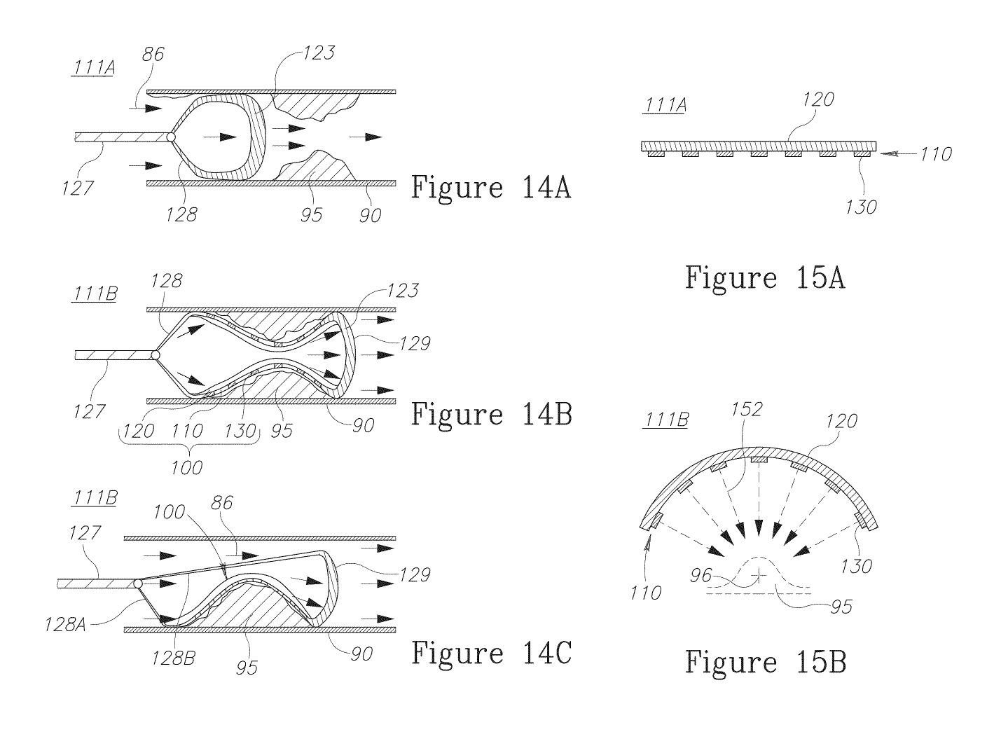

In embodiments, device 100 comprises a single layer having operable elements 130 such as openings going through the whole thickness of device 100. Such embodiment is illustrated in FIGS. 1C, 14A-E and 15A-C. In embodiments, the material of device 100 itself may be used to treat obstruction 95.

In embodiments, operable elements 130 are further configured to be mechanically de-activated upon a specified decrease of the bending, due to the treatment of obstruction 95. For example, after the size of obstruction 95 decreases due to the treatment, the treatment may be mechanically interrupted as the curvature of device 100 decreases to a certain degree, e.g. below the specified curvature threshold or below a different threshold, determined according to the structure of the surrounding tissue.

As illustrated in FIGS. 1A and 1B, device 100 is configured to bend upon contact with obstruction 95. The bending causes a mechanical activation of operable elements 130, as illustrated below. In some embodiments, operable elements 130 may comprise or be sensors. Device 100 may be hollow, and enable blood flow through cavity 85 in device 100.

The following are some non-limiting examples for curvature thresholds with respect to various obstructions. It should be noted, as illustrated below, that generally there are two dimensions related to the curvature radius. One is in the radius of vessel 90 (in a plane perpendicular to the vessel) and the other is related to the longitudinal way along vessel 90 (in a longitudinal cross section of vessel 90). As a result, operable elements 130 (such as openings 114) may be asymmetric and have different dimensions in different directions, or be oriented in different ways along device 100 (and with respect to vessel 90). Curvature thresholds may differ in different directions, as in the longitudinal axis the reference radius (that of vessel 90 without any obstruction 95) is very large, while in the cross sectional axis the reference radius (without obstruction 95) is much smaller. Hence, different activation and de-activation thresholds may be defined for these directions and may be used to apply coarser of finer treatments, possibly simultaneously in different regions or sequentially and complementary in one region of obstruction 95.

In non-limiting examples, the following may be typical parameters for the curvature threshold in cases of different obstructions. In case of coronary lesions having a radius between 0.1 mm and 2 mm, the specified curvature threshold may be about two times the lesion radius, or up to five times the lesion radius to achieve a more thorough removal of the lesion. Device 100 may be adapted or selected according to the specific lesion that is to be treated therewith.

In case of vascular lesions having a radius between 1 mm and 10 mm, the specified curvature threshold may be about two times the lesion radius, or up to five times the lesion radius to achieve a more thorough removal of the lesion. Device 100 may be adapted or selected according to the specific lesion that is to be treated therewith.

In case of airway obstruction having a radius between 0.5 mm and 5 mm for small obstructions and having a radius between 5 mm and 20 mm for large obstructions, the specified curvature threshold may be about two times the obstruction radius, or up to five times the obstruction radius to achieve a more thorough removal of the obstruction. Device 100 may be adapted or selected according to the specific obstruction that is to be treated therewith.

In case of gastrointestinal obstructions having a radius between 10 mm and 100 mm, the specified curvature threshold may be about two times the obstruction radius, or up to five times the obstruction radius to achieve a more thorough removal of the obstruction. Device 100 may be adapted or selected according to the specific obstruction that is to be treated therewith.

In case of obstructions in water or sewage pipes, having a radius between 50 mm and 500 mm, the specified curvature threshold may be about two times the obstruction radius, or up to five times the obstruction radius to achieve a more thorough removal of the obstruction. Device 100 may be adapted or selected according to the specific obstruction that is to be treated therewith.

Device 100 may be part of an arterial catheter system which includes a flexible elongate member or catheter with an outer surface, a distal region adapted to enter an artery and a proximal region extending from a patient's vessel, permitting control outside the patient's body by a physician. At the distal region of the catheter is the atherosclerosis treating assembly which may include suction and/or drug administration surface and a balloon expansion unit. Alternatively, in some embodiments it may include an atherectomy assembly which includes a cutting blade, abrasive member, snare etc. and a trapping mechanism which in certain embodiments comprises of openings in the trapping surface which extend to the proximal region of the catheter and are attached to a vacuum source. Alternatively, in some embodiments it may include an energy source like ultrasound, laser or RF.

The physician typically determines the presence and location of the plaque using one or several visualization techniques. The distal end of the arterial catheter is inserted and deployed through an incision in the femoral or brachial artery in a manner widely used in coronary and other arteries angioplasty, atherectomy and ultrasonography catheters. The catheter's distal region is advanced within the femoral or brachial artery until the distal end reaches the region of interest. Advancement of the catheter may be facilitated by X-ray fluoroscopy and the distal region of the catheter may include one or more fluoroscopic markers to enable such visualization. Advancement may also be facilitated by IVUS, TEE or by conventional guidewire and/or guiding catheter. In some cases, a distal protection unit or other filtration means will be used, either as part of the device or as a separate tool.

Once filtration/distal-protection and general lesion identification are established, the precise lesion identification mechanism is deployed. The precise lesion identification mechanism then enables the simple delivery of treatment via drug-administration, suction and/or one of several available energy sources.

FIGS. 2A-2D schematically illustrate variations of treatment application by operable elements 130. For example, operable elements 130 may comprise openings that are configured to be opened mechanically by the bending of flexible supporting layer 120 beyond the specified curvature threshold. Through the openings, drugs 135 may be eluted (FIG. 2B) or suction 136 may be applied to lesion 95. Drug elution or suction may be applied through a cavity 125 that is additional to flexible supporting layer 120 or within flexible supporting layer 120. As illustrated in FIGS. 2B and 2C respectively, device 100 may comprise a drug delivery layer 125 or a suction layer 126 in fluid communication with the openings. In embodiments (FIG. 2D), flexible supporting layer 120 itself may comprise a drug delivery volume or a suction volume in fluid communication with the openings.

The precise obstruction identification by the bending of flexible supporting layer 120 upon contact of treatment layer 110 with obstruction 95 may be facilitated by a compliant balloon 122. Balloon 122 may be inflated such that its surface takes the shape of obstruction 95. Balloon 122 may be inflated in a manner that permits blood flow while inflated. The balloon's surface may enable the administration of treatment by mechanical, electrical, chemical or other means only at the areas made concave due to the occlusion, as illustrated in the following examples.

During the operation of device 100, lesions 95 such as plaque is reduced by suction, mechanical wear, laser, RF radiation, ultrasound or drug administration. The removal of lesion material results in a reduction of surface concavity to a level in which the bending of flexible supporting layer 120 is reduced, e.g. below the specified curvature threshold. As a result, treatment of lesion 95 may be stopped and concluded. Alternatively or additionally, the occlusion status may be monitored by sensing a status of operative elements 130 (e.g. the size of openings, the position of perforators etc. as explained below) or by detecting radio-opaque marking on device 100 the outline of a radio-opaque balloon supporting device 100. When the treatment of lesion 95 (e.g. of an occlusion) is completed, device 100 is removed, e.g. by deflation of balloon 122.

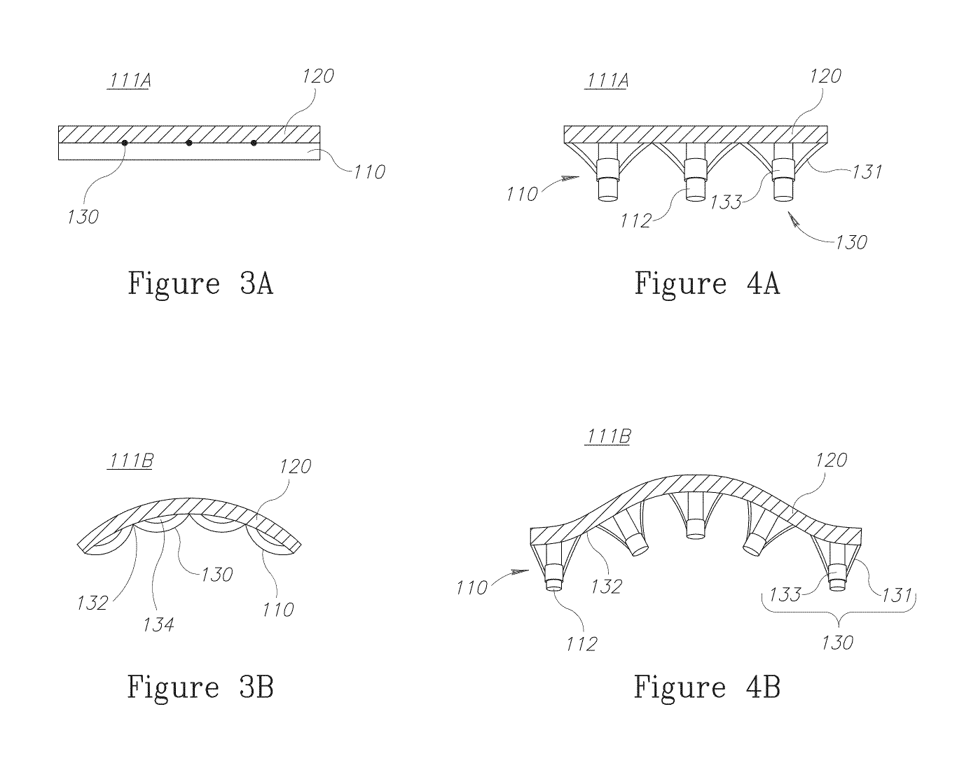

FIGS. 3A and 3B are high level schematic illustrations of device 100 configuration having a non-active state 111A and a bending-activated state 111B, respectively, according to some embodiments of the invention. In state 111B, treatment layer 110 is connected to flexible supporting layer 120 only at specified connection areas 132, leaving the rest of treatment layer 110, including operable elements 130, to be separated from flexible supporting layer 120 upon inwards bending thereof. As a result, spaces 134 are formed between treatment layer 110 and flexible supporting layer 120, which allow application of a treatment (e.g. drug elution) through operable elements 130.

FIGS. 4A and 4B are high level schematic illustrations of device 100 for perforating lesion 95, having non-active state 111A and bending-activated state 111B, respectively, according to some embodiments of the invention. Operable elements 130 may comprise perforators 133 configured to penetrate vascular lesion 95 upon bending of flexible supporting layer 120 beyond the specified curvature threshold. The perforation may be the sole treatment of lesion 95 or it may instrumental for delivering drugs to lesion 95, e.g. through openings in treatment layer 110 as described above and below, or for applying suction to fluids in lesion 95 via suction 136 as described above.

In the illustrated example, perforators 133 may be connected by supports 131 to connecting areas 132 on flexible supporting layer 120. Upon bending flexible supporting layer 120, perforators 133 may extend into lesion 95, guided by supports 131. Perforators may be hollow and extend distally to openings in flexible supporting layer 120, in drug delivery layer 125 or in suction layer 126 and function as miniature needles for drug delivery or suction. Alternatively, perforators 133 may be mounted on guides 112 for stabilization and protection in non-operative state 111A. Perforators 133 may then slide along guides 112 and extend beyond them in operative state 111B. In embodiments, guides 112 may be hollow and deliver drugs or apply suction by connection to openings in flexible supporting layer 120, in drug delivery layer 125 or in suction layer 126.

In embodiments, perforators 133 may be extended into lesion 95 by mechanical or electrical means which cause perforators 133 to penetrate the artery's inner surface to enable easy access to lesion 95. As the bending of flexible supporting layer 120 occurs only upon contact with lesion 95, all other potential administration areas remain closed and do not affected the artery's surface during the procedure. This basically serves as an “automated” procedure in the sense that it only affects the occluded areas. The holes made by perforators 133 on the surface of lesion 95 may serve as openings for drug administration, suction or mechanical mining devices designated for the treated area mainly. Referring to FIG. 4B, in the case of the mechanical apparatus, the surface's concave structure covering balloon 122 may be twisted due to side leverage by supports 131. Referring to FIG. 3B, a sliding or a radial movement between flexible supporting layer 120 and treatment layer 110 may be generated due to the varying radii of curvature of the layers. Either movement may be used, by appropriate configuration of device 100, to facilitate an opening through treatment layer 110 for application of the various treatment methods. An electrical apparatus may also be used to perforate lesion 95, by using curvature sensitive wiring to sense the concave surface of device 100 and electrically or mechanically open an opening in treatment layer 110 to access the plaque or perforate lesion 95. In embodiments, drugs 135 may be eluted or suction 136 may be applied through perforators 133.

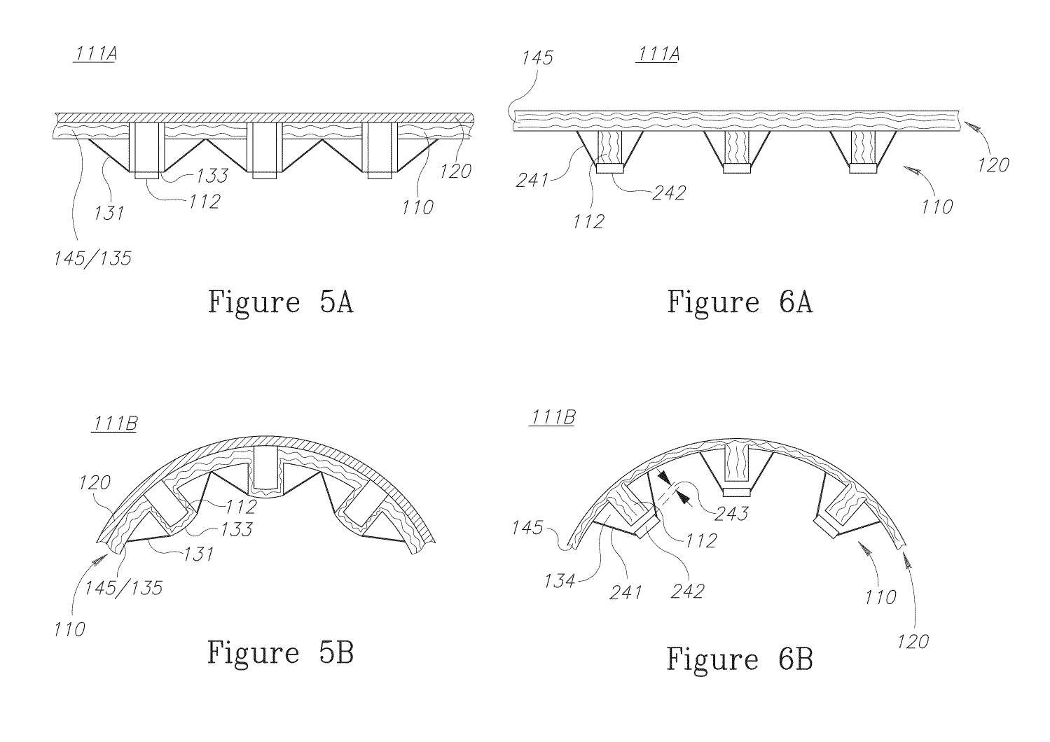

FIGS. 5A and 5B are high level schematic illustrations of device 100 for perforating obstruction 95, having non-active state 111A and bending-activated state 111B, respectively, and including an actuator 145, according to some embodiments of the invention. Actuator 145 is arranged to control mechanically the bending of device 100 and thereby to control mechanically the operation of operable elements 130. In this example, actuator 145 controls the extent to which perforators 133 are exposed (by withdrawal of tubes 112 due to the bending) and hence the depth of perforation applied to obstruction 95.

Similar actuators 145 may be incorporated in other embodiments of the invention to add a control to the extent of bending experienced by device 100 and the operation of operable elements 130. For example, actuator 145 may control the extent to which openings (see below) are opened, drugs are eluted etc. Actuator 145 may be drugs 135 themselves, that may be pressurized to control the activation of operable elements 130. Actuator 145 may incorporate sensors for the bending of device 100 that may be used to monitor the treatment. In case drugs 135 are eluted in activated state 111B, drugs 135 may be stored in non-activated state 111A, e.g. within treatment layer, instead or in addition to actuator 145 as illustrated in FIG. 5A. Alternatively, drugs 135 may be stored in non-activated state 111A in supporting layer 120 as illustrated below.

Actuator 145 may be part of treatment layer 110, of flexible supporting layer 120, or be implemented as an additional layer of device 100 at any location with respect to layers 120 and 110. Actuator 145 may be implemented as an elastic or a rigid wire, may be made of the same material as tubes 112 to control the extent of perforation resiliently or be associated with supports 131.

FIGS. 6A and 6B are high level schematic illustrations of device 100 for delivering a drug to lesion 95, having non-active state 111A and bending-activated state 111B, respectively, according to some embodiments of the invention. In the illustrated embodiment, operable elements 130 comprise openings that are mechanically controlled by the extent of bending of device 100.

Treatment layer 110 comprises tubes 112 that are covered by caps 242 in non-active state 111A. Caps 242 may be supported by supports 241 or may be connected to the edges of tubes 112 or to supporting later 120. Upon bending flexible supporting layer 120, caps 242 move to create a gap 243 with respect to tubes 112. Gap 243 may operate as an opening for eluting drugs 135. Drugs 135 may be delivered by flexible supporting layer 120, drug delivery layer 125 or treatment layer 110, the latter e.g. via spaces 134 (FIG. 6B).

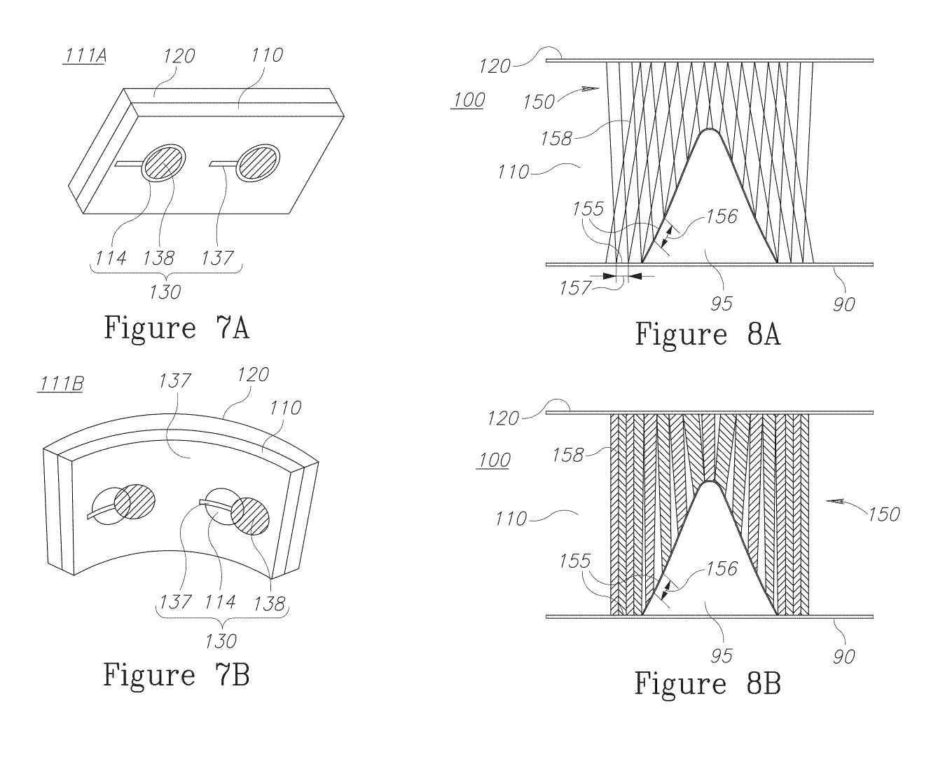

FIGS. 7A and 7B are high level schematic illustrations of device 100 for delivering a drug to lesion 95, having non-active state 111A and bending-activated state 111B, respectively, according to some embodiments of the invention. In the illustrated embodiment, operable elements 130 comprise openings 114 connected to flexible supporting layer 120, drug delivery layer 125 or suction layer 126, as explained above, and configured to elute drugs 135 or enable suction 136 there through.

In embodiments, operable elements 130 may comprise openings 114 that are covered by corresponding discs 138, configured to expose at least one of openings 114 upon the bending of flexible supporting layer 120 beyond the specified curvature threshold. Each disc 138 may be connected to treatment layer 110 or to flexible supporting layer 120 by a holder 137, that keeps disc 138 in place while opening 114 moves behind it due to the bending of flexible supporting layer 120. Upon straightening of flexible supporting layer 120, or in areas where flexible supporting layer 120 is not bend (e.g. as device 100 is not in contact with lesion 95), discs 138 cover openings 114 and prevent drug elution or suction.

The density of openings 114 on the surface of obstruction 95 may vary according to the type of obstruction, type of treatment, type and size of openings, type of mechanical activation etc. A minimum of 5-10 openings 114 per obstruction is required. In non-limiting examples, the following may be typical parameters for the density of openings 114 of different types, in cases of different obstructions.

In case of coronary lesions having a radius between 0.1 mm and 2 mm, the distance between adjacent openings 114 may be between 0.02 mm and 0.2 mm. In case of vascular lesions having a radius between 1 mm and 10 mm, the distance between adjacent openings 114 may be between 0.2 mm and 1 mm. In case of airway obstruction having a radius between 0.5 mm and 5 mm for small obstructions and having a radius between 5 mm and 20 mm for large obstructions, the distance between adjacent openings 114 may be between 0.1 mm and 0.5 mm for small obstructions and between 1 mm and 5 mm for large obstructions. In case of gastrointestinal obstructions having a radius between 10 mm and 100 mm, the distance between adjacent openings 114 may be between 2 mm and 10 mm. In case of obstructions in water or sewage pipes, having a radius between 50 mm and 500 mm, the distance between adjacent openings 114 may be between 0.1 mm and 0.5 mm. In all these application cases, the density of openings 114 in device 100 and device 100 itself may be adapted or selected according to the specific obstruction or lesion that is to be treated therewith. Some of the applications involve continuous activation of operable elements 130 over a range of curvature radii (e.g. drug elution or suction), while other applications may involve activating the operable elements discretely (e.g. energy sources activation).

FIGS. 8A and 8B are high level schematic illustrations of device 100 having a flexible network 150 with varying mesh openings 155, according to some embodiments of the invention.

Treatment layer 110 may comprise flexible network 150 and have mesh openings 155 in network 150 as operable elements 130. Network 150 is arranged to flex upon the bending to increase an opening size in a bended area of flexible network 150. For example, network 150 may be compliant to the bending by lesion 95 and designed to widen its mesh openings 155 upon the network's compliance to the lesion's form.

In the illustrated example, mesh openings 155 in the bended regions are of a width 156 that is larger than a width 157 of mesh openings 155 in non-bended regions of network 150. For example, width 156 may be three times width 157. As illustrated in FIG. 8A, network's wires 158 may be configured to be wide enough to prevent fluid communication through mesh openings 155 having width 157 but allow fluid communication through mesh openings 155 having width 156. In another example, network wires 158 may have flaps of width 157 (not shown, the wires are shown schematically to illustrate mesh openings only), that are separated from each other only when the distance between wires 158 exceeds width 157 significantly, e.g. reach width 156.

As illustrated in FIG. 8B, wires 158 may be wide enough to leave no gap 155 between them at a low radius of curvature (left side of FIG. 8B, on vessel 90), but spread apart to expose gap 155 upon bending beyond the curvature threshold due to obstruction 95 (middle of FIG. 8B, on lesion 95).

In embodiments, the space within network 150 and/or treatment layer 110 may be used to elute drugs 135 or apply suction 136 to lesion 95.

In embodiments, eluted drugs 135 may be photo activated to apply photodynamic therapy (PDT). Either or both drugs 135 and activating light may be delivered to lesion 95 via openings 114 or gap 155.

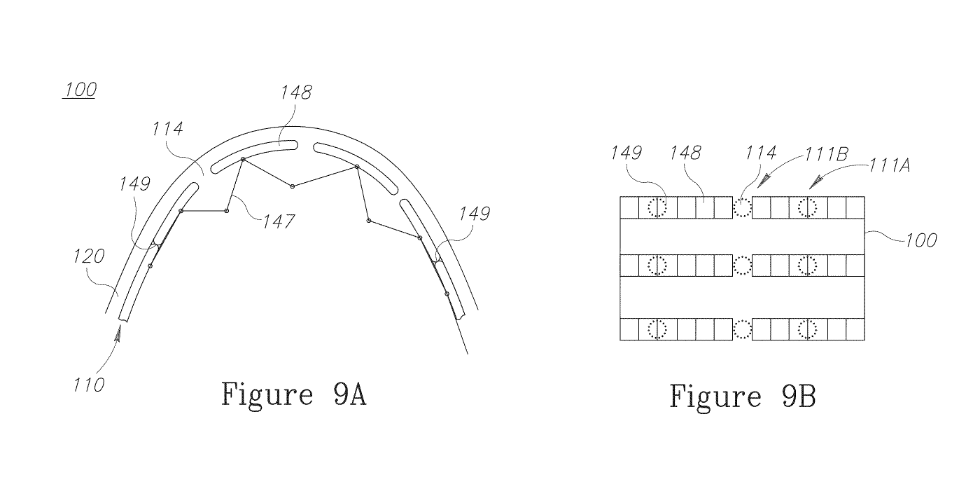

FIGS. 9A and 9B are high level schematic illustrations of device 100 for delivering drug 135 to lesion 95, according to some embodiments of the invention. In the illustrated embodiment, openings 114 in treatment layer 110 are operable elements 130. treatment layer 110 may comprise multiple segments 148 which are held together by an expandable scaffold 147 that interconnects them and allow their separation upon bending of device 100 (FIG. 9A). In a view from above, FIG. 9B illustrates the interface between operable elements 130 and obstruction 95—below the bending threshold segment 148 are close together and conceal closed openings 149, while beyond bending threshold segment 148 are spread apart to expose openings 114.

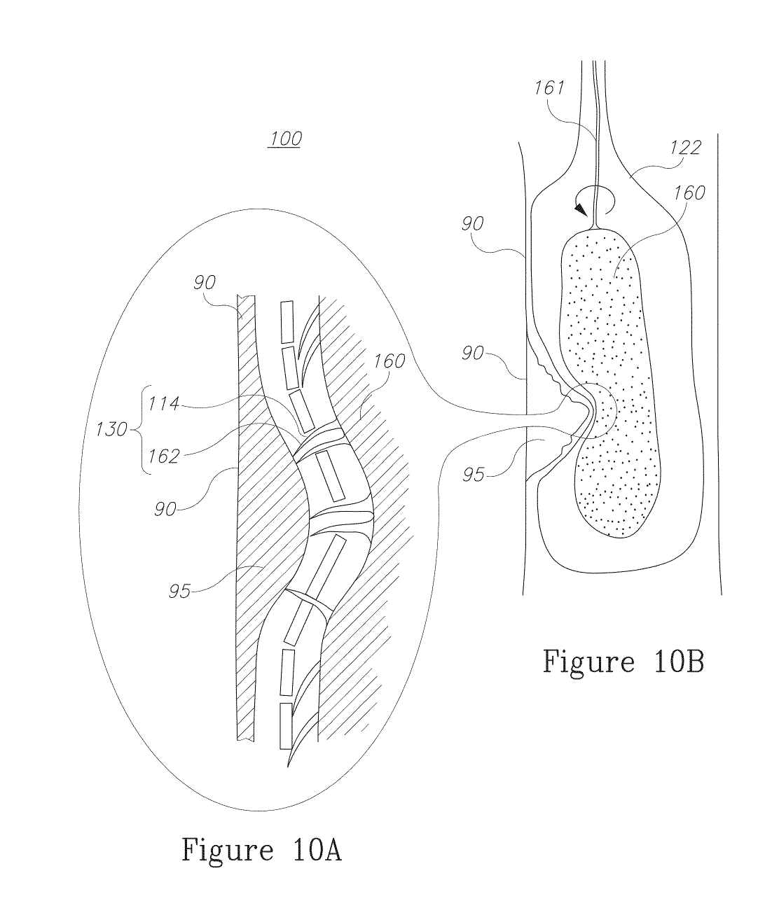

FIGS. 10A and 10B are high level schematic illustrations of an obstruction grinding device 100, according to some embodiments of the invention. Device 100 is designed to erode obstruction 95 through openings 114 in treatment layer 110.

For example, a grinder 160 may be inserted into balloon 122 and rotated via handle 161. Grinder 160 may have grinding protrusions 162 that may pass through openings 114 in activated state 111B and erode obstruction 95. Protrusions 162 may either be foldable in a deactivated state or spatially removed from layer 110 in inactive state 111A.

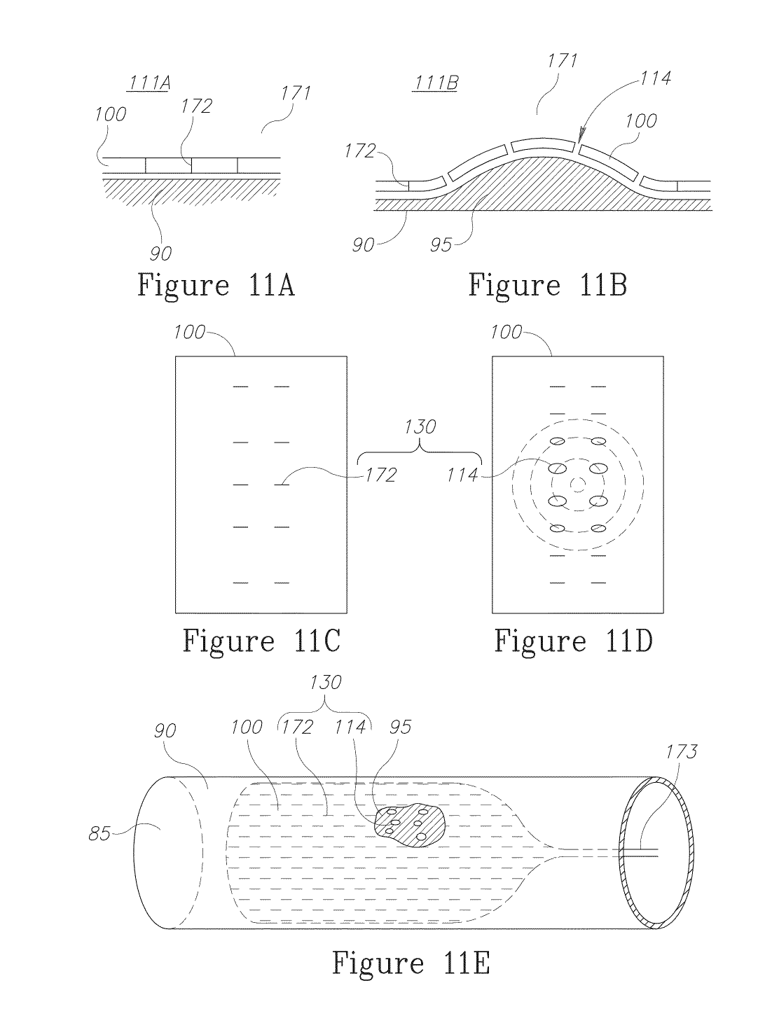

FIGS. 11A-11E are high level schematic illustrations of a one layered treatment device 100, according to some embodiments of the invention. FIGS. 11A-11E illustrate in more detail an embodiment of device 100 depicted in FIG. 1C, having openings 114 as operable elements. One layered treatment device 100 may be configured to be a part of a balloon skin, and activating openings 114 may create fluid communication between the balloon's interior 171 and obstruction 95, that may be used for drug elution or application of suction, e.g. via channel 173.

FIGS. 11A, 11C and 11B, 11D, respectively, illustrate inactive state 111A and activated state 111B, the former with slits 172 that prevent fluid communication therethrough, and the latter with openings 114 resulting from the expansion of device 100 that turns slits 172 into openings 114 and provides fluid communication therethrough, for application of the treatment.

FIG. 11E illustrates both states, the activated state over obstruction 95 and the inactive state over level areas of vessel 90. The bending of device 100 over obstruction 95 yields the stretching and expansion of device 100 that turns slits 172 into openings 114 and provides fluid communication therethrough, for application of the treatment.

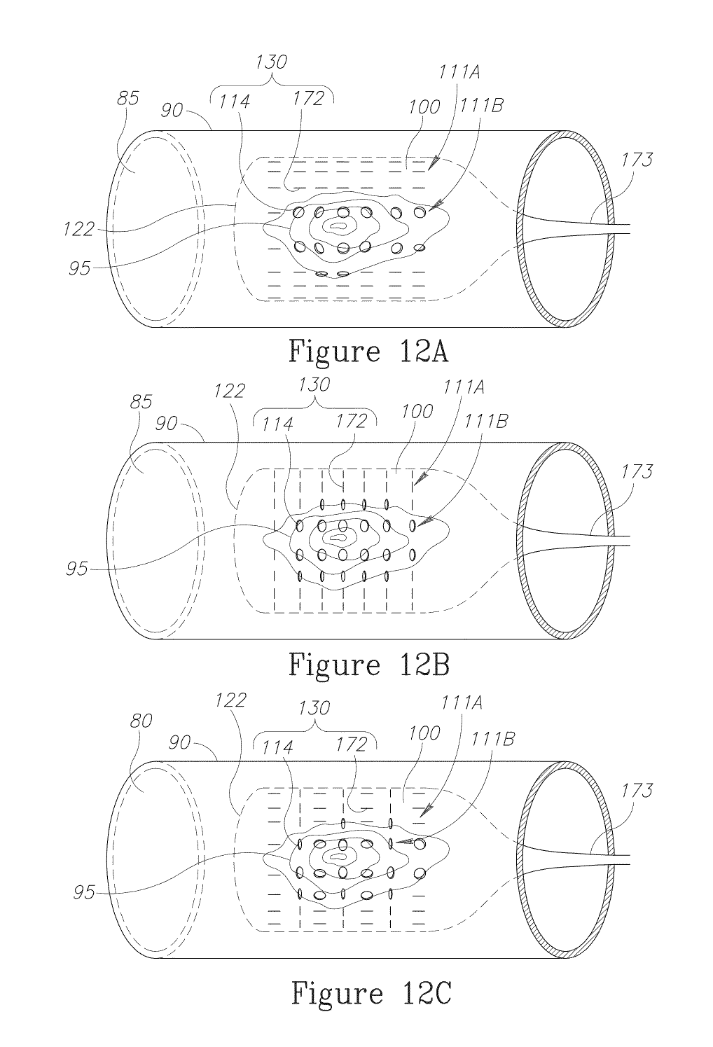

FIGS. 12A-12C are high level schematic illustrations of openings 114 as operable elements, according to some embodiments of the invention. FIG. 12A illustrates a design of device 100 (one or two layered) with slits 172 and corresponding openings 114 along the direction of vessel 90 (and of flow 85 along vessel 90). FIG. 12B illustrates a design of device 100 (one or two layered) with slits 172 and corresponding openings 114 that are perpendicular to the direction of vessel 90 (and of flow 85 along vessel 90). FIG. 12C illustrates a design of device 100 (one or two layered) with a mixed configuration of slits 172 and corresponding openings 114, some of slits 172 along the direction of vessel 90 and some perpendicular thereto.

The direction of slits 172 influences and allows spatial control of the mechanical activation of operable elements 130 (in the illustrated case openings 114, but not limited thereto). In particular, the specified curvature threshold is defined with respect to slit direction—the radius of curvature of obstruction 95 must decrease below the given threshold curvature radius along a direction that is perpendicular to slit 172 to induce the maximal opening thereof. A direction of radius of curvature decrease that is tangential to the direction of slit 172 generates much less spreading of opening 114, if at all (depending on the threshold values). Hence, the direction of slits 172 and openings 114 may be selected according to the local topography of obstruction 95 to apply the selected treatment. Any mixture of slits 172 having different orientations induces a different level of activation of operable elements with respect to a given obstruction topography, and hence allow a spatially differentiated treatment of obstruction 95, i.e. certain regions in obstruction 95 may be treated at different intensities, or a given region may be treated at different intensities sequentially by different areas of device 100.

In embodiments, the material of device 100 itself may be used to treat obstruction 95. For example, device 100 may be implemented as balloon 122 or as the parachute itself and activate the treatment upon bending of device 100 as balloon or parachute skin upon obstruction 95.

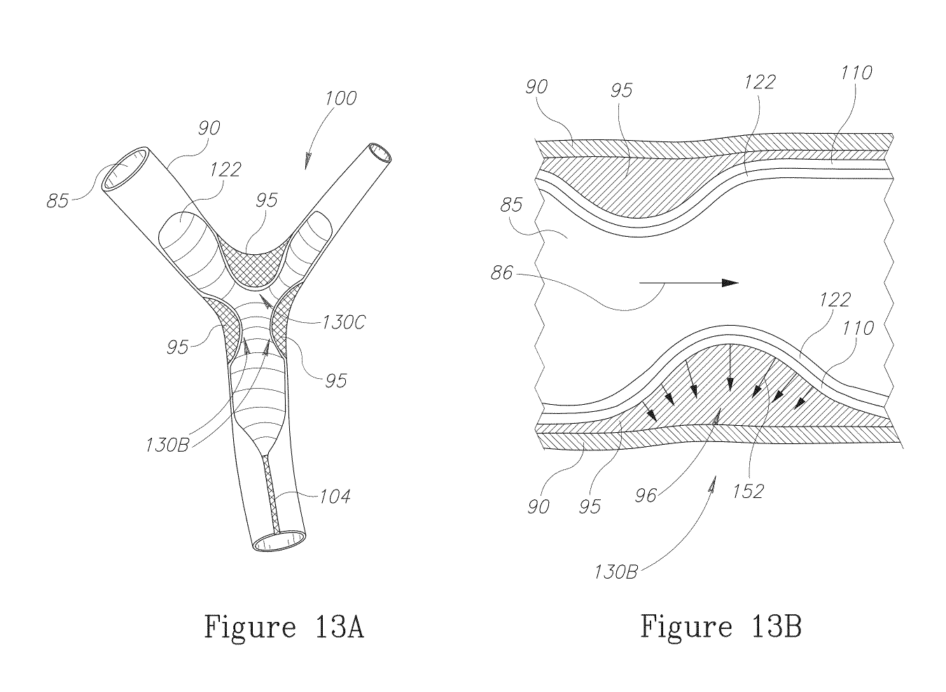

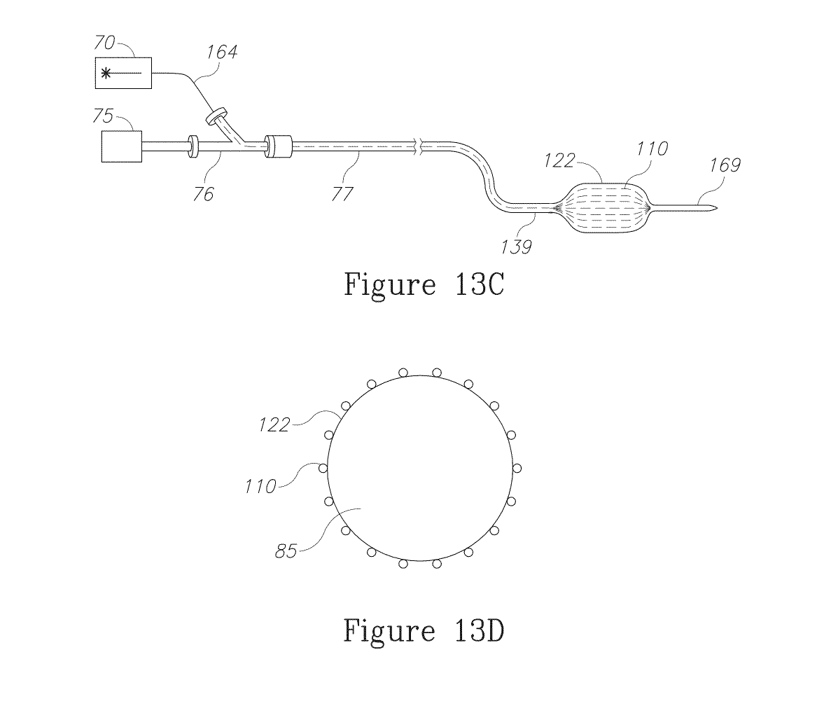

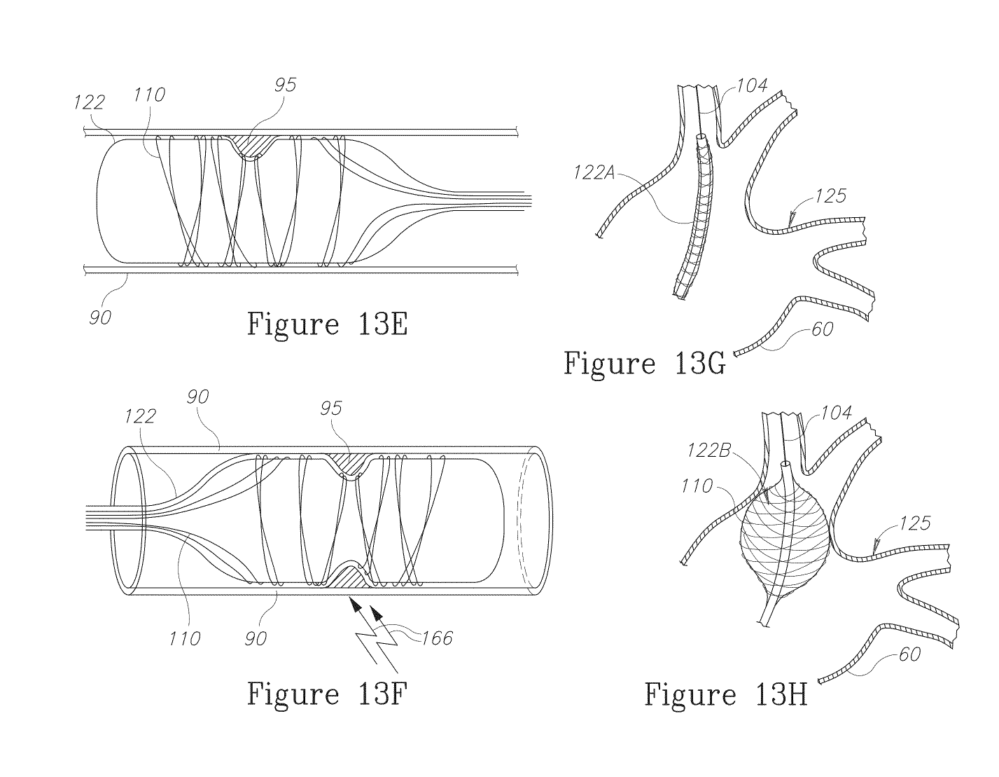

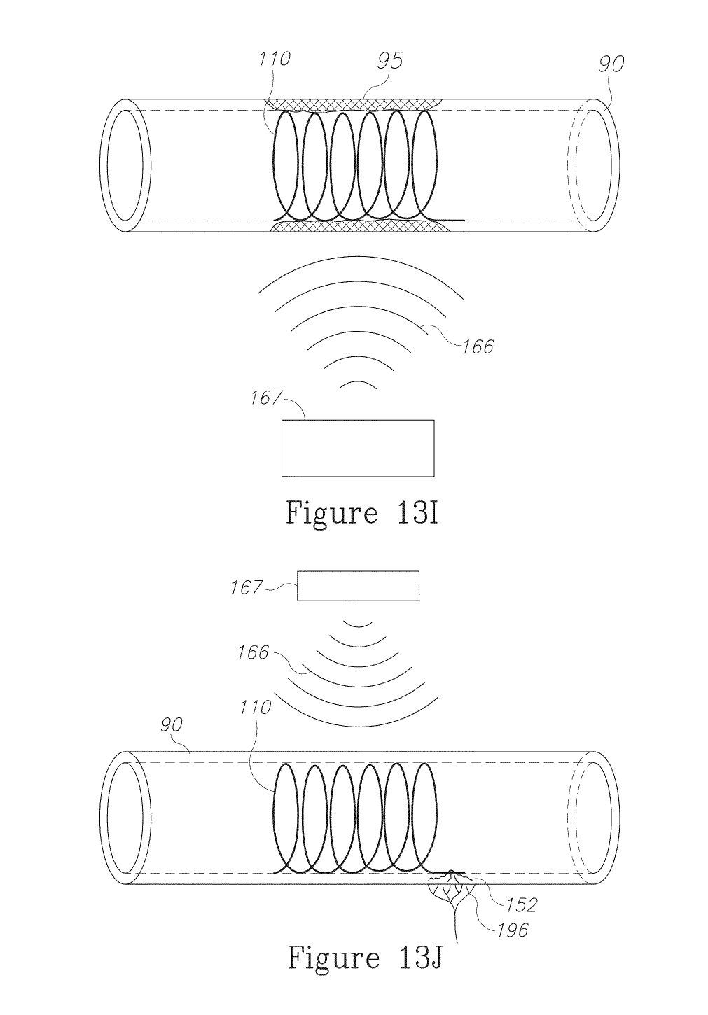

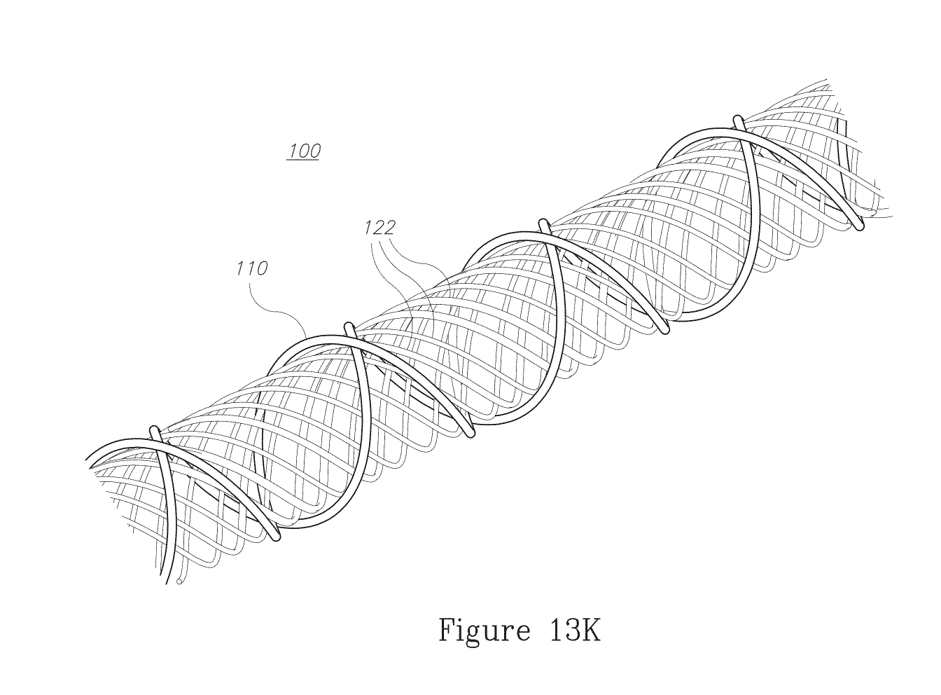

FIG. 13A is a high level schematic illustration of balloon implemented device 100 for treating lesions 95 at vessel bifurcations, according to some embodiments of the invention. FIG. 13A illustrates an embodiment with fibers 110 wound transversely to the direction of application of balloon 122

Device 100 may be configured as balloon 122 (see also FIG. 2A for a hollow balloon, allowing flow such as blood flow through cavity 85 in the balloon) with flexible supporting layer 130 being part of or externally attached to a skin of balloon 122. In such embodiment, the bending of flexible supporting layer 120 is carried out by inflating balloon 122 against vascular lesion 95 and device 100 thus utilizes the lesion's natural curvature to activate operable elements 130.

Balloon 122 may be connected to a delivery system 104 that is in fluid communication with drug delivery layer 125, suction layer 126 or flexible supporting layer 120 to elute drugs 135 or apply suction 136.

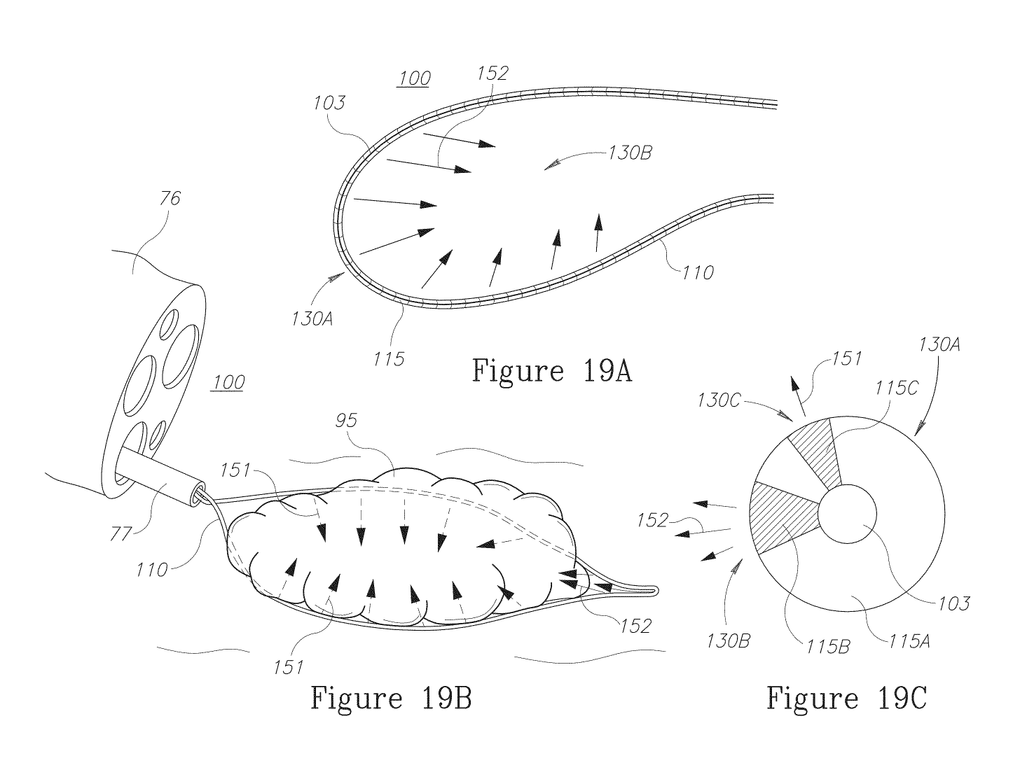

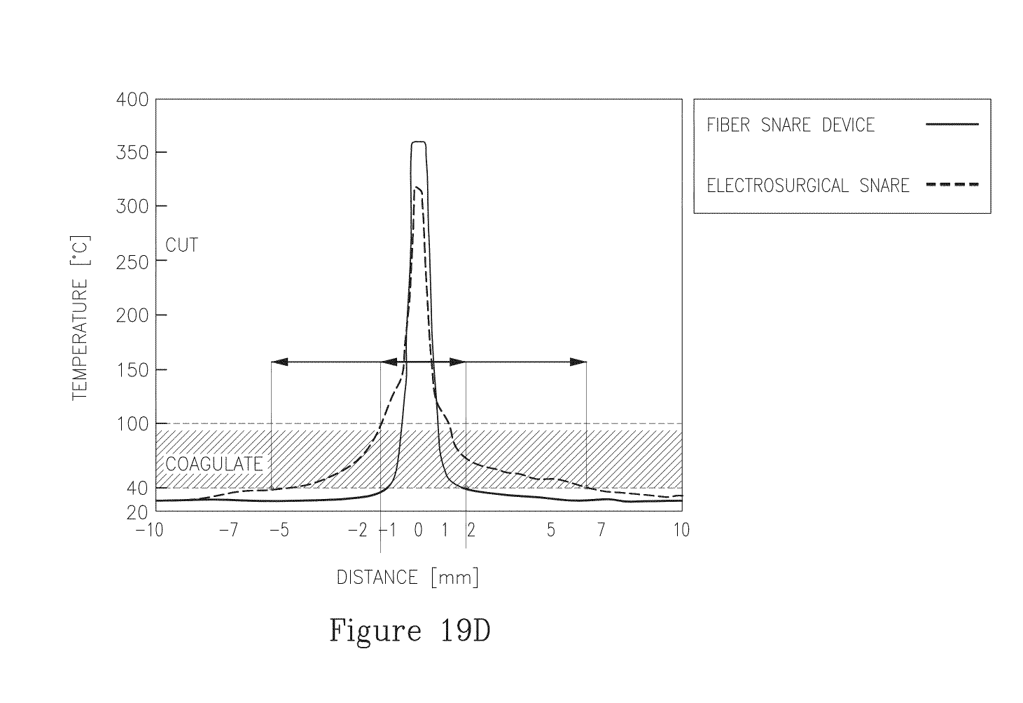

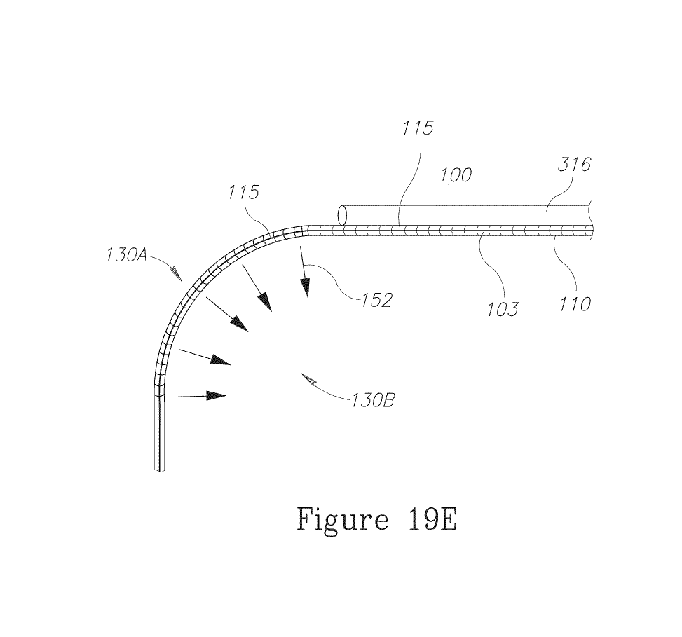

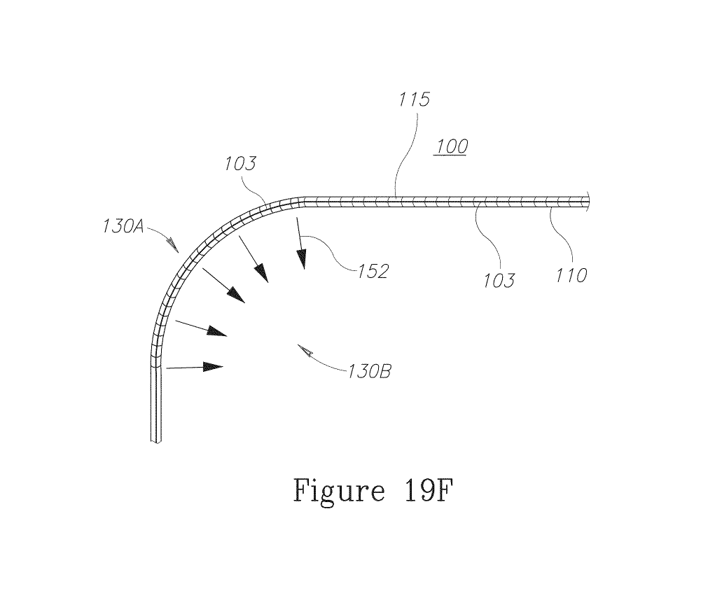

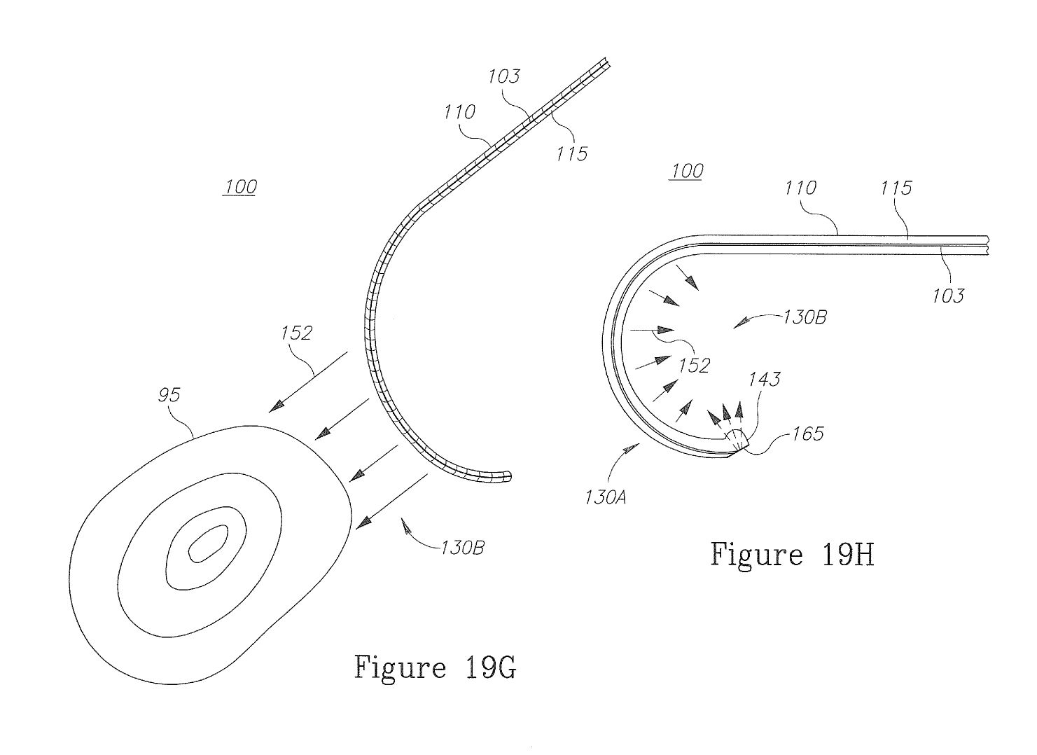

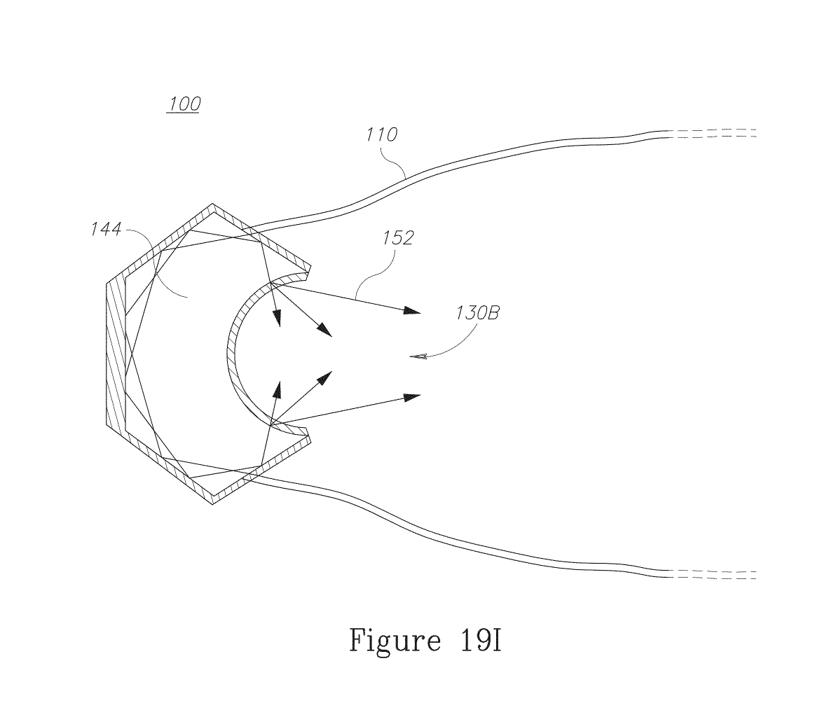

FIG. 13A illustrates flow obstructions 95 such as vascular lesions in an arterial bifurcation. While prior art treatment of lesions in bifurcations is particularly difficult due to the geometry of the treated area, device 100 utilizes the difficult geometry to enhance the treatment, as the bending of balloon 122 and fibers 110 attached to it is enhanced by the difficult geometry and thus facilitates treatment. In embodiments, removal of obstructions 95 may complete the treatment simply by the resulting increase in the bending radius beyond the specified threshold. Device 110 clearly allows treatment of several obstructions, as illustrates in FIGS. 19F and 13A. Lesion 95 is located at sides of vessel 85 and at the apex of the bifurcation. Emission regions 130B, 130C may be multiple, and fibers 110 may be configured to emit radiation based on different thresholds in regions 130B, 130C and thus enable specialized treatment of the bifurcation. For example, parameters of different emission regions may be determined by geometrical considerations of target 95 and operation surrounding and its relation to the structure of device 100 as well as by treatment considerations (types of applied treatments at different regions, radiation intensity, safety considerations etc.).

Furthermore, while vessel bifurcations are especially hard to treat with customary devices (because the prior art device must be shaped according to the bifurcation in advance), device 100 is particularly efficient is such cases as the curvature is enhanced by the bifurcation. In addition, device 100 is particularly efficient in treating the apex of the bifurcation, as the prior art disadvantage of the apex lesion having a small curvature radius become an advantage for the present invention, which utilizes the small curvature radius to enhance activation and treatment efficiency.

FIG. 13B illustrates a device for treating flow obstructions and treatment of flow obstructions 95 by a supportive structure 122 that allows continued flow 86 through lumen 85, such as of a vessel 90 (e.g., an artery), according to some embodiments of the invention. Bending of fibers 110 attached to supportive structure 122 applies radiation 152 from operable elements 130 (represented by arrows) as treatment of obstructions 95, possibly focused 96, and upon their removal, the bending flattens and treatment ceases.

FIGS. 13C-13K are high level schematic illustrations of device 100 having optical fibers 110 exhibiting bend emission, that are wound on a supportive structure 122 such as a compliant or non-compliant balloon, according to some embodiments of the invention.

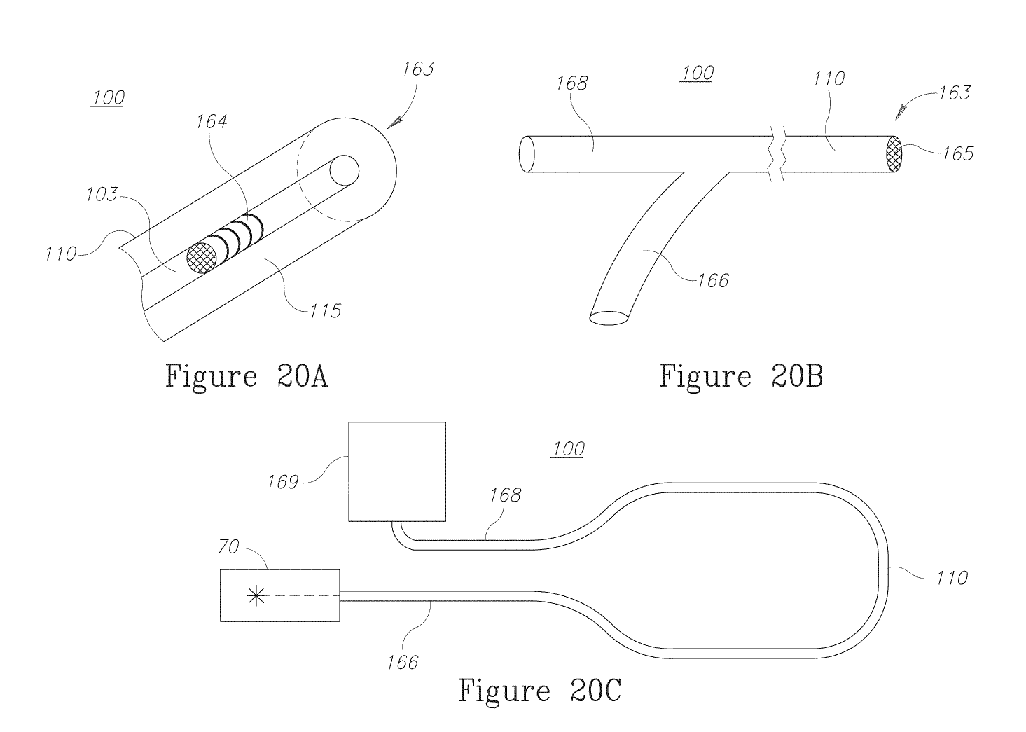

FIG. 13C illustrates supportive structure 122 embodied as balloon 122, to which fibers 110 are attached. Balloon 122 may be inflated by pump 75 and inserted via delivery system 76 and channel 77, such as an endo scope, possibly guided by a guide wire 138 (see FIG. 13G). Balloon 122 may be fixated and controlled by a guide 104 close thereto. Balloon 122 may comprise absorptive sink 169 at its end, or wound fibers 110 may be circular and guide back excessive radiation (as illustrated schematically in FIG. 20C).

Fibers 110 may be wound and attached to balloon 122 in different configurations. FIG. 13D illustrates a balloon cross section with fibers 110 evenly distributed on the skin of balloon 122. FIGS. 13E, 13F, 13I and 13J illustrate more complex winding patterns involving spiral windings in two directions. Winding of fibers 110 may be transverse or longitudinal with respect to the length axis of balloon 122 or exhibit any combination thereof or other form. In general, optical fibers 110 may be wound in a configuration that is perpendicular, parallel or oblique to a longitudinal axis of balloon 122 or in a combination thereof.

Winding of fibers 110 may be stent-like, having a collapsible stent-like or balloon-like configuration 122A (FIG. 13G) and an expanded stent-like or balloon-like configuration 122B (FIG. 1311). FIGS. 13I and 13J may also be implemented in a stent-like design. FIGS. 13I and 13J illustrate implanted stents 100 comprising active fibers 110 which upon energy transfer 166 from a source 167 within or outside the body emit energy 152 through bended emission regions 130B (not shown in this figure). FIG. 13I illustrates device 100 which may be implanted in a region of obstruction 95 such as plaque or other diseased region in e.g. the esophagus (Gastroesophageal reflux disease GERD) or bronchi (asthma), to enable gradual ablation of plaque/tissue 95, or localized treatment by drug activation PDT. For example, one or more fibers 110 may be incorporated in a stent of any kind to deliver radiation that activates a drug that is associated with the stent. Furthermore, device 100 may comprise in any of the embodiments, a drug eluting means such as a needle, arranged to elute a drug in the vicinity of the radiation emission. The radiation may be then utilized to activate the drug or the drug may be used to enhance the effect of the laser treatment. FIG. 13J illustrates device 100 in which emitted energy 152 may be used to inhibit or activate nerve firing 196, for example in the renal arteries to block sympathetic outflow from the kidney and thus reduce systemic blood pressure, to block pain signals in certain cases, or to control the sensation of hunger in eating disorders or obesity. Such states may also be treated by fibers 110 and devices 100 illustrated in other embodiments. FIG. 13K illustrates device 100 comprising fibers 110 wound around a stent 112 or replacing one or more of the wires of braided stent as supporting structure 122. Bended fiber 110 may be arranged to emit radiation that supports the placing of stent 122, ablates tissue surrounding stent 122 (e.g. before, during or after the healing process) or interacting with eluted drugs.

In embodiments, any optical fiber may be used in combination with a stent for activating a treatment such as ablation, activation of a radiation sensitive drug or activation of nerve endings for various purposes. Regular emission from fiber tips may be used in addition to or in place of bend emission to deliver the required radiation and apply specified treatments.

Supportive structure 122 of device 100 is configured to position optical fibers 110 with respect to a target 95 (such as a flow obstruction) in a way that causes bending of all, some or at least one optical fiber 110 beyond the specified bending threshold thereof over target 95, to emit electromagnetic radiation 152 at target 95. In the case of balloon 122, inflating balloon 122 may cause fibers 110 to bend upon contacting target 95 beyond the emission threshold.

The topographically induced bending can be utilized to deliver energy specifically to those areas where the treatment is required, such as flow obstructing plaque (see e.g., FIGS. 13A and 13B). Balloon 122, which may be delivered with a standard catheter delivery system, may be inflated in-situ, causing fibers 110 on the surface of balloon 122 to be pressed against the artery and plaque and be imprinted with the topography. The imprint causes bends in the fiber that induce the desired bend emission which is used to treat the lesions.

FIG. 13F illustrates an embodiment of device 100 that is operated by an external source 166. External source 166 may activate device 100 by any kind of radiation or field applied to device 100. Such device 100 may have a balloon as supportive structure 122, or fibers 110 may be stent-like, having a collapsible stent-like configuration (FIG. 13G) and an expanded stent-like configuration (FIG. 1311). Device 100 may be used acutely during procedure or be implanted for permanent use.

In embodiments, fibers 110 may be arranged as resonators that amplify induced radiation from source 166 and thus generate the required radiation for treatment without using an internal light source 70.

FIGS. 13G and 13H illustrate a collapsed configuration and an expanded configuration of device 100 (configured either as a balloon or as a stent) in treating an obstruction in a heart 60. Such embodiments may comprise a stop point for fibers energy or an energy sink, as illustrated e.g., in FIGS. 13C and FIGS. 20A-20C below.

FIGS. 14A-14C are high level schematic illustrations of a parachute implemented device 100 in stages of operation, according to some embodiments of the invention. The parachute form allows utilizing patterns of fluid flow 86 (e.g. blood flow through arteries) to deploy device 100 upon obstruction 95 by utilizing the flow distracting characteristics of obstruction 95, in addition to utilizing the form of obstruction 95 for applying the treatment itself. Device 100 is configured to contact obstruction such as vascular lesion 95 by being carried along by blood flow 86 until being obstructed by vascular lesion 95.

In embodiments, parachute 123 comprises a holder 127 connected via strings 128 to device 100 and configured to guide device 100 to bend upon obstruction 95 by utilizing fluid flow patterns in the vicinity of obstruction 95. In embodiments, parachute 123 may further comprise a frontal leading area 129 configured to maintain a force resulting from fluid flow 86 and acting to position device 100 on obstruction 95. In embodiments, leading area 129 may be part of device and may be used to apply treatment. Using parachute 123 further ensures maintaining fluid flow 86 through vessel 90 during the treatment, which may be an essential requirement, e.g. when treating lesions in the carotid arteries.

In embodiments, the parachute may be replaced by a torus shaped balloon (not shown) that allows blood flow therethrough. The operation of the torus shaped balloon may similarly utilize blood flow to place device 100 upon the lesion, or generally utilize fluid flow to place device 100 upon obstruction 95.

FIGS. 14B and 14C illustrate examples for deployment of parachute implemented device 100, in a case of opposing lesions 95 or bilateral narrowing of vessel 90, and in a case of a single lesion 95, respectively. In the former case (FIG. 14B), strings 128 may stabilize device 100 on both lesions 95 while frontal leading area 129 may ensure an appropriate coverage of lesions 95 by device 100. In the latter case (FIG. 14C), strings 128 may act asymmetrically to stabilize device 100 on lesion 95 while frontal leading area 129 may ensure an appropriate coverage of lesions 95 by device 100. A string 128B farther from lesion 95 may expand or be released to allow a far end of device drift further and cover lesion 95, while a string 128A closer to lesion 95 may shrink or be kept short to place a close end of device onto the beginning of lesion 95. Such asymmetric operation utilizes fluid flow 86 to place device 100 onto lesion 95 and utilize its bending to apply the treatment.

Parachute 123 may be deployed by first releasing device 100 into the blood stream far enough from lesion 95, such that flow is not affected by lesion 95. Second, device 100 is deployed such that regions of device 100 that are in low flow or low flow resistance regions are anchored onto lesion 95 and regions of device 100 that are in high flow regions are not restricted. In this way device 100 tends to physically cover and deploy itself over the regions of lesion 95 which obstruct flow 86 the most. Parachute 123 may then be designed to include a therapeutic agent, an electric source, an ultrasound source or have light energy conducting properties to facilitate treatment by operable elements 130 at lesion 90 and prevent treatment or protect adjacent healthy regions. Furthermore, parachute 123 may be anchored to a catheter system for deployment and consequent retraction after treatment.

In embodiments, device 100 further comprises a flow sensor (not shown) for measuring the flow patterns near lesion 95. The flow measurements may be used to plan the application of parachute implemented device 100.

In embodiments, holder 127 may further comprise delivery system 139 in fluid communication with drug delivery layer 125, suction layer 126 or flexible supporting layer 120 to elute drugs 135 or apply suction 136.

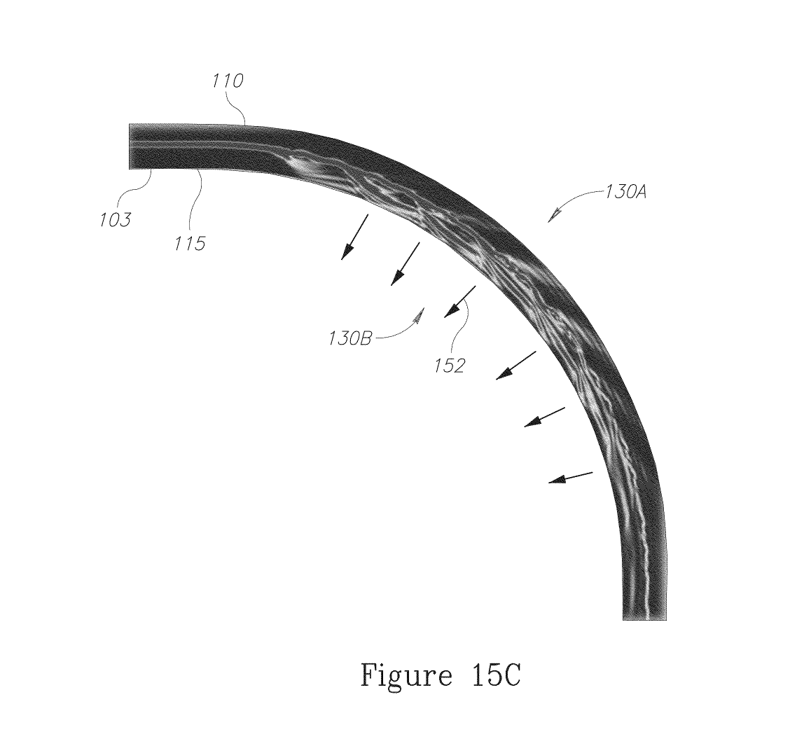

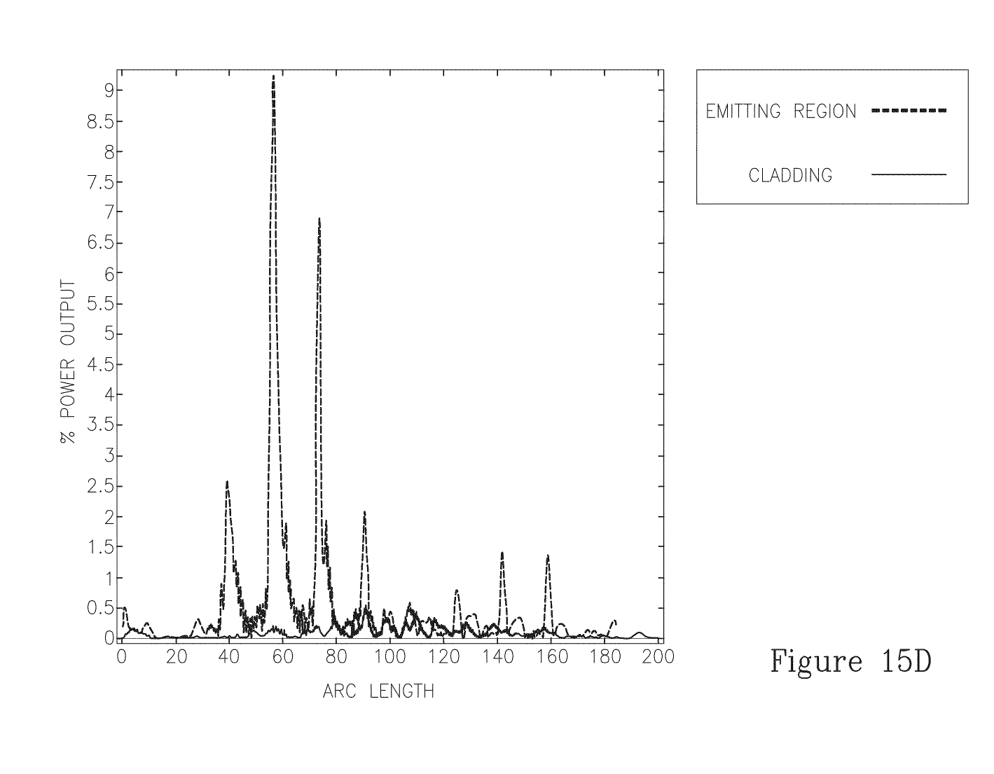

FIGS. 15A and 15B are high level schematic illustrations of device 100 for focusing energy at obstruction 95, having non-active state 111A and bending-activated state 111B, respectively, according to some embodiments of the invention.

In embodiments, operable elements 130 may comprise energy sources configured to deliver energy to vascular lesion 95. For example, the energy sources may deliver electromagnetic radiation to obstruction 95 such as laser energy. Operable elements 130 may comprise the energy sources themselves or optical elements that are configured to direct and deliver the electromagnetic radiation from an external energy source (not shown) to obstruction 95. In another example, the energy sources may deliver mechanical energy, e.g. in the form of ultrasound pressure waves, or electric energy, e.g. in the form of current. Operable elements 130 may comprise the energy sources themselves or transducers that receive energy from an external source (not shown).

The energy sources may be activated by the bending or may radiate at a low level that doesn't harm the surrounding. The effective energy level may be reached only upon adding several sources by the bending. Alternatively or complementary, the energy sources may be covered by treatment layer 110 and exposed to obstruction 95 upon widening of openings 114 in the cover, as described in other embodiments.

The concavity of device 100 may be used as a focusing mechanism of the energy sources. Emitted energy 152—from multiple operable elements 130 such as openings or bending regions of treatment layer 110 such as an energy delivering device, e.g., an optical fiber 110—may be concentrated due to the bending of device 100 at a focal point 96 on lesion 95. Curving treatment layer 110 over obstruction 95 points the energy sources to the same point 96 in the plaque body (in case of a vascular lesion), e.g., to the center of the plaque buildup.

Operable elements 130 may be positioned at such spaces and orientations that, upon the bending of flexible supporting layer 120 at a certain curvature, operable elements 130 deliver energy 152 to specified focal areas 96 on obstruction 95. In this way, energy 152 from different energy sources is added and may be configured to be beyond a specified treatment threshold. In such a configuration, energy 152 delivered from any single operable elements 130 (such as an opening in an energy delivering device, or bending regions of respective fiber 110) may be selected to be harmless to surrounding tissue (in state 111A), while upon concentration of several energy sources, due to the bending of device 100 on obstruction 95 (in state 111B), an effective treatment is applied. In embodiments, operable elements 130 may comprise sensors that detect the bending of obstruction 95 and activate an external energy source (not shown) to deliver energy to operable elements 130 only upon detection of obstruction 95.

FIGS. 15C and 15D demonstrate bend emission for the fiber configuration illustrated in FIG. 1711 (see below, serrated fiber), according to some embodiments of the invention. FIGS. 15C and 15D thus illustrate an embodiment of operable elements 130 as features of fiber 110 as treatment layer 110 or as part thereof. In certain embodiments, operable elements 130 may be embodied in fiber 110 as treatment layer 110 by various means, such as configuration of any of coating 140, cladding 115, emitting regions 130A, 130B, core 103 or supporting structures 122. Operable elements 130 as configurations of fiber(s) 110 may be associated with different supportive layers 120, as illustrated in any of the embodiments disclosed herein, and elements of supportive layers 120 may be configured to support the configuration and/or operation of fiber based operable elements 130.

FIG. 15C is a direct photograph of transmitted light in fiber 110, showing emission through the bended side 130B of fiber 110. Generally speaking, the mechanical tension in the bent fiber lowers the refractive index on the external side and increases the refractive index on the inner side, to generate bend emission as a combined result of the geometry and material influence of the bending.

FIG. 15D illustrates the power output as percent of the transmitted radiation from the specified region upon bending (broken line) in comparison to emitted radiation through cladding 115A outside emitting sector 130B (solid line). Clear emission peaks are evident at an arc length range of 40-160 μm of the circular fiber cross section, i.e. in the specified bended sector.

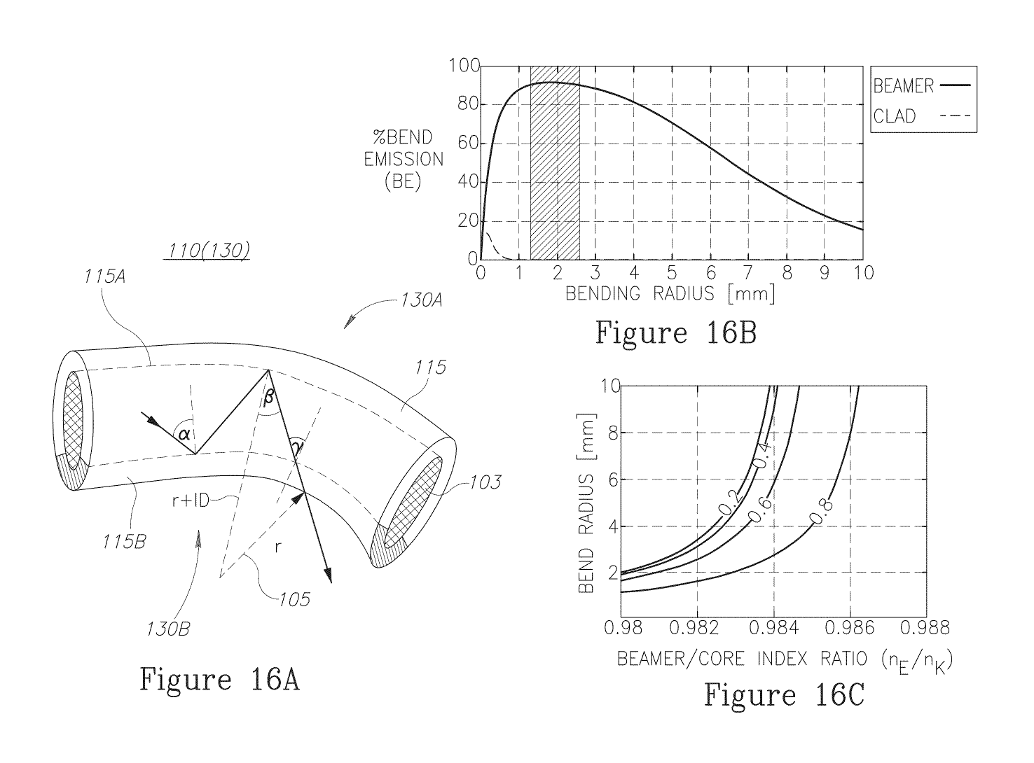

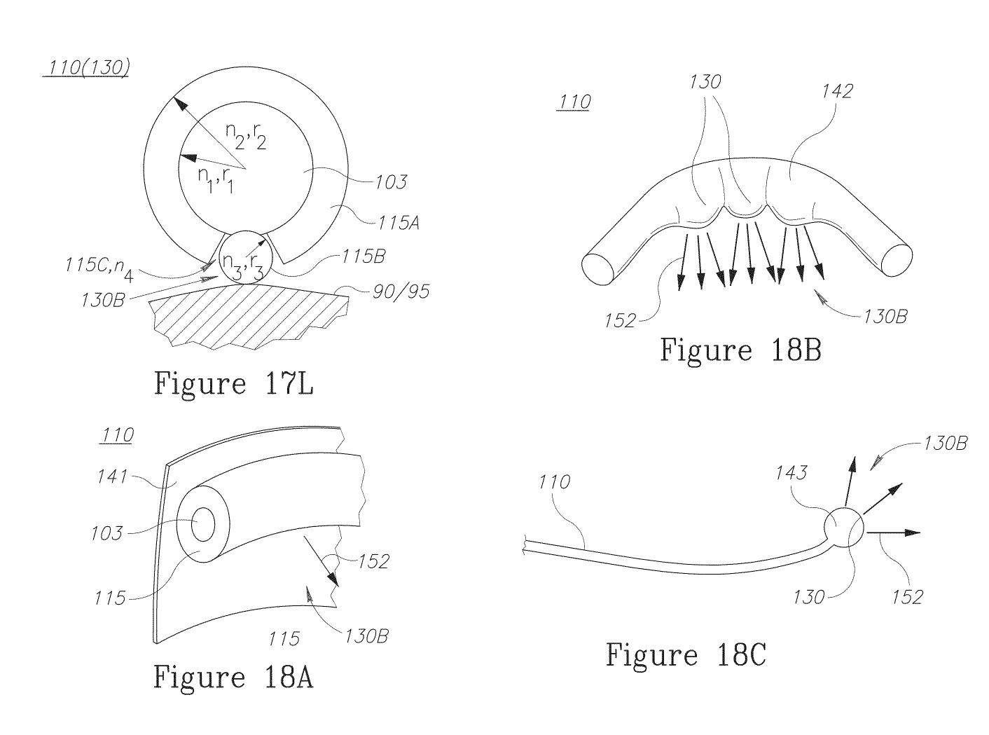

It is noted that operable elements 130 as illustrated in FIGS. 15A and 15B may be implemented in many of the embodiments which illustrate bending regions 130B of optical fibers 110 in following illustrations (as non-limiting examples, FIGS. 18B, 19A, 19E, 19G, 22A, 23B, 27B, 27C, 28A-C). As explained below (FIGS. 16A-C and 17A-K), implementation of energy emitting operable elements 130 as emitting fiber regions 130B may be used in various implementations. However, other disclosed embodiments of operable elements 130 in such implementations are likewise included within the scope of the present invention.

FIG. 16A is a high level schematic block diagram of an emitting region of an optical fiber 110, according to some embodiments of the invention. Optical fiber 110 comprises a core 103 having a refractive index n_{K} and a cladding 115 having a refractive index n_{M}. Optical fiber 110 has at least one specified region of cladding 115 that is arranged to emit electromagnetic radiation from core 103 upon bending optical fiber 110 at the specified region beyond a specified bending threshold with respect to a bending radius r 105. The emission of electromagnetic radiation from core 103 upon bending optical fiber 110 is related to in the following as bend-emission (BE). The bend emission depends on various fiber and radiation characteristics such as the size, structure and materials of the fiber, bending radius 105, radiation frequency and so forth. The fiber is designed to enable bend emission in the specified region and sectors only, while continuing to prevent transmission through the cladding in other parts of the fiber.

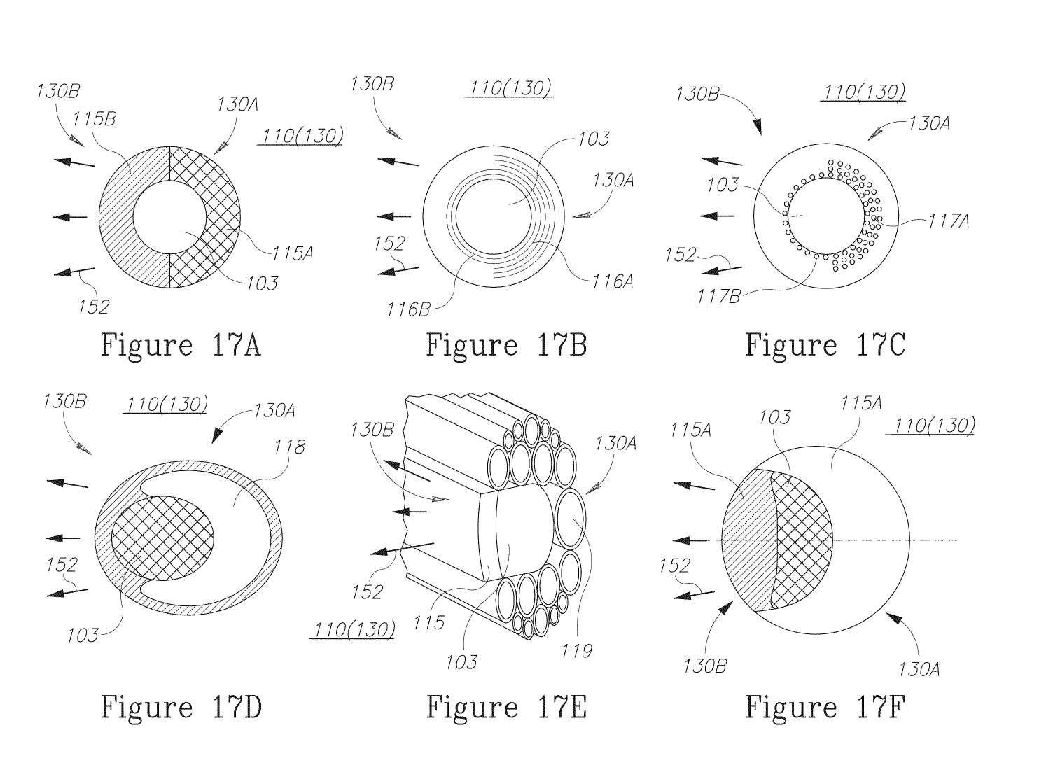

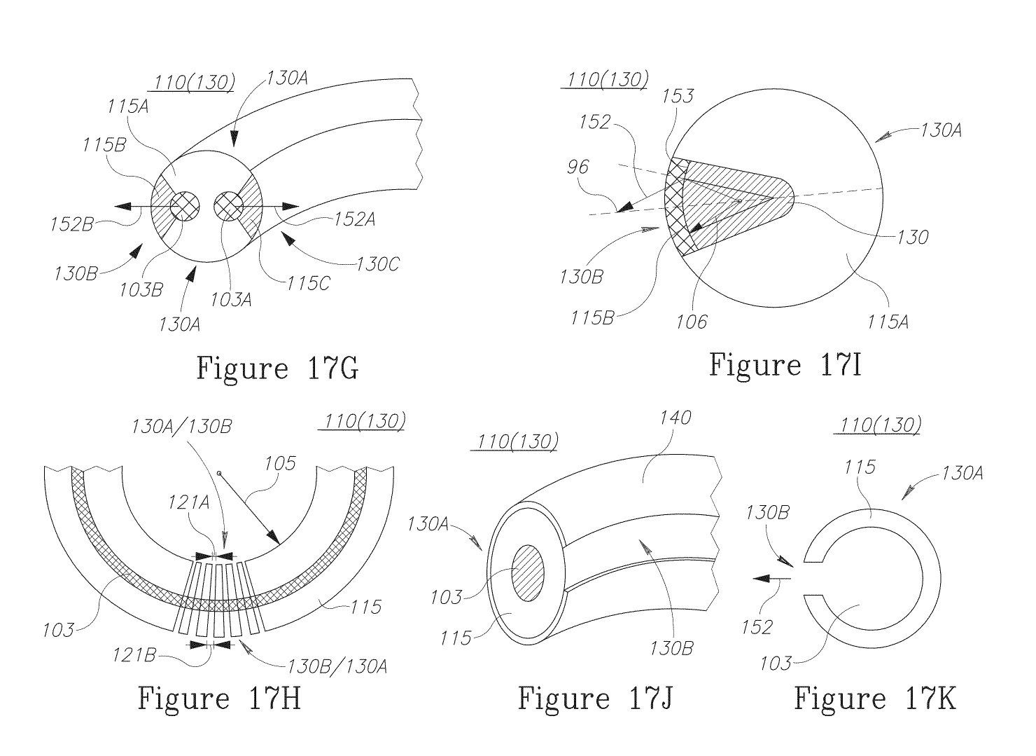

It is noted that bend-emission may be configured to occur inwards or outwards with respect to the direction of bending. It is further noted that the disclosed principles are also applicable to other types of waveguides, e.g., RF waveguide (see e.g., FIG. 17K illustrating a metallic waveguide), which may be tailored for specific geometrical parameters allowing highly controlled and specific emission patterns.

Bend emission may be achieved by bending fiber 110 prior to an actual application thereof, e.g. bending fiber 110 to have a snare-like form, and angled form, a stent-like form etc. (see examples below), and then controlling the bend emission by the light source upon placing the bended regions of fiber 110 in an operative position. Alternatively or complementary, bend emission may be under geometrical control, achieved by making use of the natural curvature of the targeted object to generate the desirable energy discharge profile from the waveguide. Certain regions in fiber 110 may be designed to bend-emit upon curving in contact with the target, as exemplified below, and the energy that is emitted in bends in these regions is actually used to achieve the desired goal. In such case, emission may be controlled by the actual bending, in addition or in place of controlling the light source.

Any type of fiber 110 may be arranged to emit radiation upon a specific bending, e.g. a waveguide (which may comprise metallic waveguides), a solid core optical fiber, a hollow fiber and a photonic crystal fiber (such as a holey fiber, a Bragg fiber or any other micro-structured fiber). The non-emitting sector(s) may be micro-structured (e.g. with a grating or air holes) to reduce an effective refractive index thereof below a refractive index of the emission sector and/or to direct radiation toward the emission sector.

Optical fiber 110 may be single-mode or multi-mode, in the latter case, the specified emission region and bending threshold may be selected with respect to the required modes, to control the emitted energy. In addition, the specified emission region and bending threshold may be selected with respect to, and controlled by, the beam polarization.

Bends in fiber 110 that may be used in emitting regions 130B include both micro-bends (local deviations from the fiber's linearity, with relative small bending radii) and macro-bends (changes of angle of the fiber's direction, usually larger bending radii). For example, the emitted radiation from a macro-bend may be estimated, for single mode fibers, by the expression: Exp (8.5−519·D·(λ_{ce}/(2λ·MFR))³) in dB/m, where D is the bending radius in mm, λ is the wavelength in μm, λ_{ce} is the fiber cut-off wavelength in μm and MFR is the mode fiber radius in μm.

FIG. 16A illustrates the condition for bend emission, according to some embodiments of the invention, by illustrating an example of a possible trajectory of light travelling down fiber 110. The light reaches the beginning of the bended specified region 130B at an angle α that is not yet sufficient for BT due to the bend radius which is not sufficiently small at this point (i.e. a is still larger than θ_{emitting region}=sin⁻¹(n_{E}/n_{K})). The light is thus reflected to cladding side 115A and is internally reflected at angle which is larger than θ_{cladding}=sin⁻¹(n_{M}/n_{K}), and stays within core 103. Upon reflection from cladding 115A the light reaches sector 130B with cladding 115B at an angle γ which is now smaller than θ_{emitting region}=sin⁻¹(n_{E}/n_{K}), as the bending radius reaches at this point the threshold radius and goes beyond a specified bending threshold of the specified region of fiber 110 (i.e. bending radius becomes smaller than the threshold radius). The transmitted light exits region 130B at an angle θ. The exact calculation follows Wang et al. (2007) “Investigation of Macrobending Losses of Standard Single Mode Fiber with Small Bend Radii”, in Microwave and optical letters 49:9, 2133-2138. Bend-emission can be approximated as starting to occur at angle θ_{bend} that is defined using bending radius r 105 as: θ_{bend}=sin⁻¹(r_{bend}(r_{bend}+ID)), ID being the internal diameter i.e. core diameter. The condition for bend-emission is that sin⁻¹(n_{M}/n_{K})=θ_{cladding}<θ_{bend}<θ_{emitting region}=sin⁻¹(n_{E}/n_{K}).

Other than the prior art, the present invention utilizes conditional and controllable side emissions from an optical fiber. In contrast to side firing fibers, fibers of the present invention do not emit any radiation when straight or bended below the bending threshold. The side emission is activated only upon the bending of the fiber at a predetermined bending radius, for example by an obstruction that is to be removed by the fiber, or according to a specific device design.

During treatment by emitted radiation 152, parts of the treated target (e.g. a flow obstruction or a polyp) are removed, causing the target to be decimated and flattened. In some embodiments, target flattening reduced the bending of fiber 110 (increases the bending radius thereof) and causes a reduction in bend emission until conclusion of the treatment. Such effect may be desired and taken into account when selecting the bending threshold. In some embodiments, a different specified region may take over the treatment, and be activated by a different bending threshold to allow multi-stage treatment.

FIG. 16B is a schematic illustration of the dependency of the emitted radiation through the specified region on bending radius 105, according to some embodiments of the invention. FIG. 16B illustrates a broad peak of emitted radiation at about 1-3 mm of bending radius, in which over 90% of the electromagnetic radiation transferred through optical fiber 110 is emitted through the specified region. Furthermore, FIG. 16B illustrates that radiation is emitted from cladding 115 upon bending, not specifically from the specified region (dashed line), is much smaller in intensity and occurs at much smaller bending radii (mostly under 0.5 mm). Hence, FIG. 16B illustrates the good controllability of the emitted radiation by the design of the specified region.

In one example, the specified region has a cladding 115A having a refractive index n_{M} in a non-emitting sector 130A and a cladding 115B having a refractive index n_{E} in an emission sector 130B. In case of a solid core fiber, the refractive indices satisfy n_{K}≧n_{E}>n_{M}. In case of a hollow (air) core fiber, the refractive indices satisfy n_{M},n_{E}>n_{K}. In the latter case, embodiments may comprise either n_{M}≧n_{E} or n_{E}≧n_{M} depending on the indices of refraction and on the material's absorption, scattering and micro-structure.

FIG. 16C is a schematic illustration of the dependency of the emitted radiation through the specified region on bending radius 105 (y axis) and on the ratio n_{E}/n_{K} (x axis), according to some embodiments of the invention. The lines in FIG. 16C indicate ratios of electromagnetic radiation emitted through the specified region to electromagnetic radiation transferred through optical fiber 110, namely 0.2, 0.4, 0.6 and 0.8, which are also the values indicated on the y axis of FIG. 16B. FIG. 16C illustrates increasing emission with n_{E}/n_{K} ratio nearing 0.99 (with constant radius 105) and increasing emission with decreasing bending radius 105 (with constant n_{E}/n_{K} ratio), i.e. with stronger bending of optical fiber 110.

In embodiments, one or more optical fibers 110 may be incorporated in a device 100 (see e.g. FIGS. 20C, 13A and 13C) having at least one light source arranged to transmit electromagnetic radiation through one or more optical fibers 110. The following description starts with embodiments of optical fiber 110 and continues with embodiments of device 100. For the sake of brevity of description, it is understood that any one of the optical fiber embodiments may be implemented in any one of the device embodiments, and all possible combinations are included therefore in the present invention.

FIGS. 17A-17I are high level schematic illustrations of various embodiments of operable elements 130 of optical fiber 110 having an asymmetric cladding 115 in cross section, according to some embodiments of the invention. FIGS. 17A-17I illustrate cross sections through the specified region. Generally, operable element(s) 130 as emitting sector(s) is numbered 130B (and 130C, in case of several emitting sectors) while the non-emitting sector is numbered 130A. The respective coating sections are numbered 115B and 115A respectively. FIGS. 17A-17I illustrate one or two emitting sectors as examples, operable element(s) 130 of fiber 110 may be arranged to comprise more than two emitting sectors 130B, 130C, multiple different sectors at different specified regions along fiber 110 and any other region and sector configuration according to given requirements.

FIG. 17A illustrates fiber 110 with two differing cladding types 115A, 115B having different refraction indices n_{M}, n_{E} respectively. n_{E} is generally closer to n_{K} so that bending fiber 110 at the specified region beyond the bending threshold results is emission 152 of radiation through section 130B in the specified region, as exemplified in the calculations below.

In embodiments, the normalized refractive index difference between the cladding of the specified emission region and the cladding of non-emitting regions may be larger than ca. 0.1% (e.g. as Δ=(n_{E}−n_{M})/n_{E}), and may be larger than ca. 0.5%. Differences may be larger or smaller, depending on the exact materials and structures used and operational (predefined or resulting) bending radii 105 (as a non-limiting rule of thumb, the larger the difference in refractive indices, the smaller is the threshold bending radius).

Two non-limiting examples, which refer to commercial SiO₂ fibers having a refractive index of n_{K}=1.457 (at 633 nm) are (i) n_{M}=1.456 and n_{E}=1.457 and (ii) n_{M}=1.000 and n_{E}=1.450.

FIG. 17B illustrates fiber 110 with two differing cladding types 115A, 115B having different types of microstructures 116A, 116B respectively, in the illustrated cases different Bragg type structuring of cladding 115. In the illustrated example, Bragg structures 116A are more extensive in cladding 115A than Bragg structures 116B in cladding 115B, resulting in emission 152 through sector 120B upon bending beyond the threshold. Other embodiments may comprise one sided Bragg micro-structuring or multiple zones with differing Bragg micro-structuring. Furthermore, structure 116B may have no Bragg layers and may be constructed of a single material.

FIG. 17C illustrates fiber 110 with two differing cladding types 115A, 115B the latter having microstructures 117A, 117B such as holes (e.g. air holes), that reduce n_{M} in non-emitting region 130A with respect to n_{E} in emitting region 130B. In general, optical fiber 110 may be an asymmetric photonic crystal fiber, which can also be used for additional purposes. Optical fiber 110 may be an asymmetric Photonic Crystal Fiber (PCF), e.g. according to one of the illustrated embodiments, which can also be used for additional purposes. Microstructures 117A, 117B may be present in emitting region 130B and in non-emitting region 130A to different extents (regarding the number of microstructures 117A, 117B, their parameters and their spatial expansion), or be present on one side only, e.g. only microstructures 117A in non-emitting region 130A, to prevent emission therefrom.

FIG. 17D illustrates fiber 110 having cladding 115 designed to have a large air gap 118 in non-emitting region 130A and additionally core 103 that may be asymmetrically positioned (i.e. off-center or eccentrically) with fiber 110. Core 103 may further be non-circular (e.g. elliptic in FIG. 17D) to define the bending threshold according to requirements. The form and extent of air gap 118, the specific design of cladding 115 and of core 103 may be adapted to specific bend-emission parameters and specifications.

FIG. 17E illustrates another asymmetric fiber configuration, comprising asymmetric core 103 positioned off-center of fiber 110 and cladding 115 comprising multiple air holes 119 arranged to define non-emitting region 130A. The exact configuration of core 103 and cladding 115 may be selected according to specific bend-emission parameters and specifications.

FIG. 17F illustrates another example of asymmetric fiber 110, namely an embodiment having a specified form and position of the emitting sector 115B of cladding 115 and of core 103.

FIG. 17G illustrates optical fiber 110 having multiple emission sectors, that may be designed at various positions in the cross section of fiber 110. In the illustrated case as a non-limiting example, two opposite emission sectors 130B, 130C, that may have similar or different emission characteristics. Emission sectors 130B, 130C may be at different angles to each other and may comprise more than two emission sectors. Emission sectors 130B, 130C may be associated with different cores 103B, 103A respectively, e.g. to be configured to emit electromagnetic radiation 152B, 152A with different parameters (e.g. wavelength, intensity). Emission sector 130C may be associated with additional core 103A. Emission sectors 130B, 130C may have different bending thresholds, also in respect to the direction of emission (may emit upon inwards or outwards bending).

Electromagnetic radiation, such as laser energy, may be delivered to either or both cores 103B, 103A simultaneously or sequentially. Without loss of generality, fiber 110 may have several specified regions and/or several emission sectors 130B, 130C etc. which may be constructed axially so as to deliver energy along fiber 110 to different specified regions and/or the emission sectors may be constructed tangentially to deliver energy to several regions along fiber 110 simultaneously or sequentially.

FIG. 17H illustrates optical fiber 110 having radially serrated cladding 115 at the specified region, in which spaces 121A, 121B between the serrations are defined according to the specified bending threshold. Upon bending fiber 110, serrations on one side of fiber 110 move closer to each other (spaces 121A become smaller with respect to a straight fiber 110), while serrations on the opposite side of fiber 110 spread apart (spaces 121B become larger with respect to a straight fiber 110). The cladding serrations may thus be configured to define distinct operable elements 130, as illustrated in FIGS. 15A-D. Operable elements 130 as emitting region 130B may be on either side of fiber 110, depending on the relation between n_{K} and n_{M}. For n_{K}>n_{M} (solid core), the crowding together of the serrations increases the effective refractive index of cladding 115 to near n_{K} and emitting region 130B is hence on the concave side, while the spreading apart of the serrations reduces the effective refractive index of cladding 115 and non-emitting region is hence on the convex side of fiber 110.

The serrations on the concave side can guide the energy in a periodic manner (see FIG. 15C), focusing energy to desired regions along the specified region. FIG. 15D (see above) illustrates the energy delivery in a simulated 90° bend in optical fiber 110. The energy delivery through the cladding is in solid line and the radiation through the specified region is in dashed line. The emitted radiation is focused periodically due to the serration in the concave bended region. In addition, serrations also enable mechanically the bending of fiber 110 in smaller bend radii.

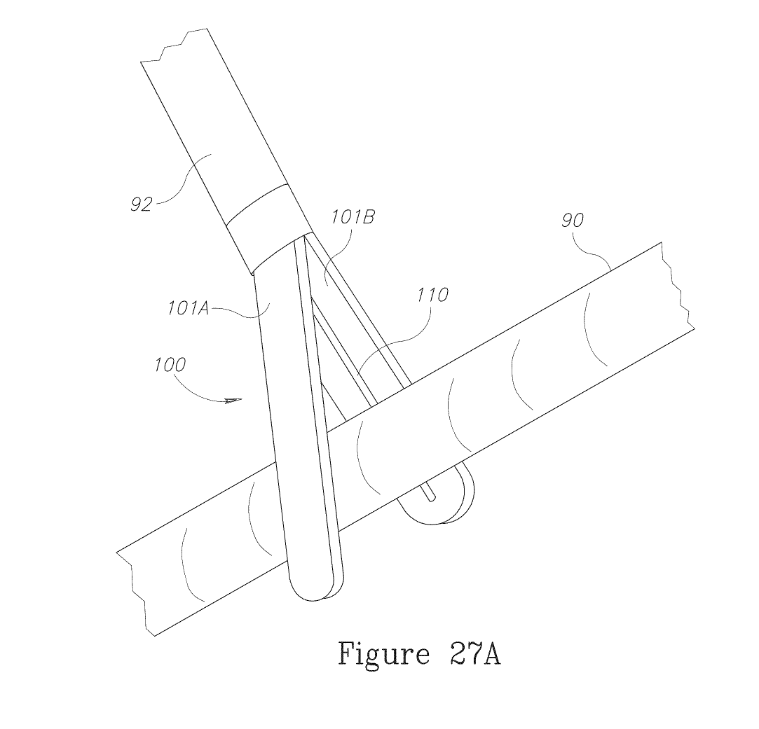

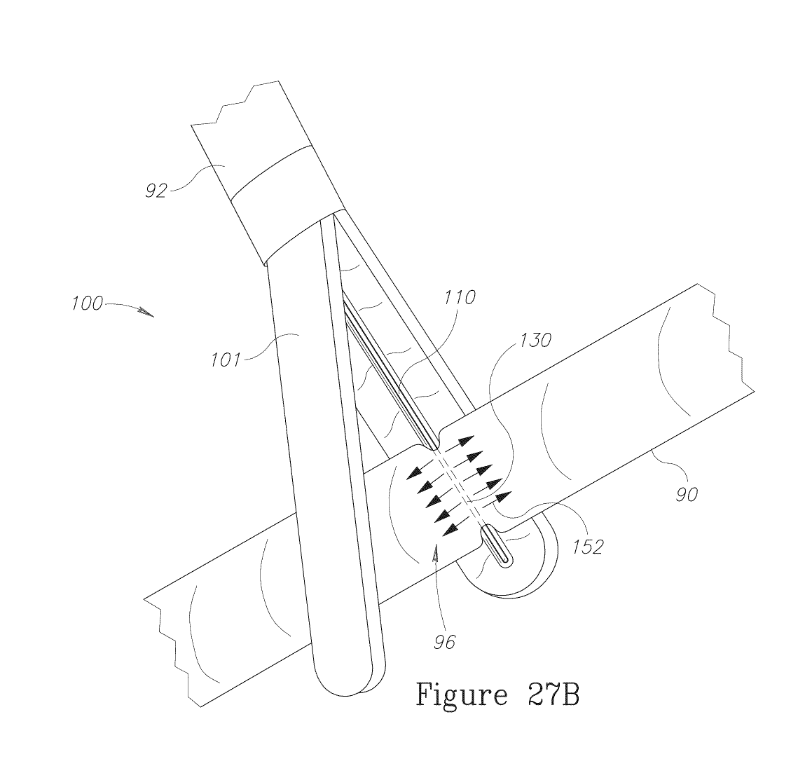

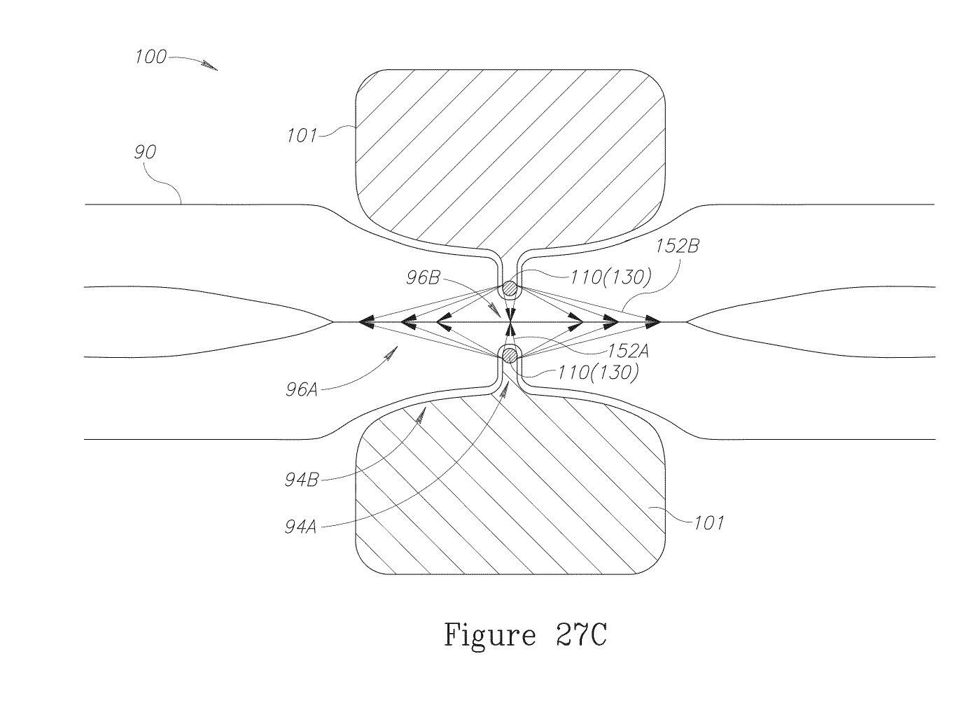

FIG. 17I illustrates another example of asymmetric fiber 110, namely an embodiment having a specified form and position of the emitting sector 115B of cladding 115 and of core 103. Core 103 is elongated and eccentric relative to cladding 115, and has an effective radius 131. The cross sectional structure may be designed to direct emitted radiation 152 to focal point 96 (see also FIGS. 15B, 27C, etc.), e.g., on a radial symmetry axis of emitting sector 115B. A core-cladding interface 153 may be shaped to influence the emission characteristics.

The fiber's cross section may be configured to emit radiation 152 not only in a specific direction but also in a specific profile. The light from core 103 may be optically manipulated to exit the waveguide in a narrow flat profile or alternatively in a dispersive profile as needed. Core-cladding interface 153 may be designed to serve as a lens to focus the naturally dispersive energy. For example, FIG. 17I illustrates a cross-sectional view of one of the profiles that serves to focus the energy. In this case, trajectory 153 of emitted radiation 152 reaches core-cladding interface 153 and is then focused inward to focal point 96 closer thereto than would have been emitted radiation from a radially symmetric interface, due to the shorter effective radius 131 of core 103. The unique assembled structure allows to asymmetrically emit the energy away from core 103 to one side only and also manipulates interface 153 in a very specific manner that dictates the exact emission profile. Other profiles may be generated by fiber cross sectional profiles exemplified in FIGS. 17D-17F.

The index of refraction also depends on temperature. Generally, an increase of temperature causes an increase in the index of refraction. In one embodiment, in which fiber 110 is inherently asymmetric as in FIG. 17I, energy discharge 152 may be asymmetric as well. Since energy 152 is discharged on the inner side of the bend, this side gets warmer, creating a positive feedback loop which increases discharge 152 and thus the accuracy and safety as well since this effect occurs only at the treatment site. The increase of temperature leads to an increase in the index of higher refraction in emitting section 130B and thus to increased energy discharge and more heating, reinforcing the discharge mechanism. Straightening the bend halts and reverses the process. The precise target energy of the tissue's cutting/heating is tuned to enhance and increase the effectiveness of bend emitted radiation 152. The cladding material may be chosen with specific absorption to control the effect of bend-emission.

FIG. 17J illustrates an embodiment of fiber 110 with a coating 140 that has a gap or is missing at specified emission region 130B, according to some embodiments of the invention. Coating 140 may have an inner reflective surface that yields or enhances the bend emission and its directionality. Generally, coating 140 may be asymmetric in cross section at specified region 130B. Other parts of fiber 110 or the device (see below) may be configured to enhance beam emission.

FIG. 17K illustrates an embodiment of a metallic waveguide having a gap at specified emission region 130B, according to some embodiments of the invention. The design and applicative concepts for fiber 110 and device 100 that are presented herein, may be applied to metallic waveguides, delivering e.g. electromagnetic radiation. The gap in the metallic waveguide may be used to emit radiation 152 therethrough for applying a treatment.

FIG. 17L illustrates an embodiment of fiber 110 with an emitting region 115B at specified emission region 130B, according to some embodiments of the invention. Emitting region 115B is connected at the vertical dimension with core 103 of fiber 110 and may contact tissue 90 and/or 95; and is separated at the horizontal dimension from the rest of cladding 115A, e.g., by an air gap 115C. The configuration of dimensions r₁, r₂, r₃ and width of gap 115C and of refractive indices n₁, n₂, n₃, n₄ may be selected to yield side emission at specified conditions, such as upon bending fiber 110. In a non-limiting example, r₁=100 μm n₁=1.445, r₂=150 μm n₂=1.42, r₃=50 μm n₃=1.438 and n₄=1 may be used to yield required bend-emission under specified conditions. In certain embodiments, fiber 110 may be configured to have substantially r₂=r₁+r₃ and n₄<<n₂<n₃<n₁ to yield effective side emission upon bending. In certain embodiments, the refractive indices may be selected according to the following relations: n₁>1.005·n₂; 0.999·n₁≧n₃>1.001·n₂ and n₄<0.99·n₂.

FIG. 18A is a high level schematic illustration of fiber 110 with a supportive structure 141 attached on an opposite side of fiber 110 with respect to emitting region 130B and the target, according to some embodiments of the invention. Supportive structure 141 may be used to orient fiber 110 to position emitting region 130B at a correct orientation with respect to the target and reflect excessive energy upon the target. Supportive structure 141 may be associated in various embodiments of device 100 (see below).

For construction purposes, fiber 110 may be attached to or embedded in reinforcing structure or stabilizer 141, to ensure stability and prevent breakage. Reinforcing structure 141 may be a semi-tube (e.g., similar to coating 140 in FIG. 17J) or a ribbon (e.g. as in FIG. 18A), made of plastic or metal. In some cases fiber 110 may be inserted into stabilizer 141 after it is set in the desired location, for example, after stabilizer 141 has been inserted around obstruction 95 (e.g., a polyp or a tumor) to be excised. Furthermore, reinforcing structure 141 may serve for inward reflection of emitted energy 152.

FIG. 18B is a high level schematic illustration of fiber 110 with lateral circle segments 142, according to some embodiments of the invention. Segments 142 have arcs that define the emission sectors and further emit electromagnetic radiation 152 at a plane defined by the arcs. Fiber 110 may comprise one or more such segments 142 at the specified region. Segments 142 may be part of cladding 115, e.g. produced by cutting into cladding material, be part of coating 140 or be attached structures. Segments 142 may further focus radiation 152 onto the target.

FIG. 18C is a high level schematic illustration of fiber 110 with a circular segment 143 such as a disc, at the fiber tip, according to some embodiments of the invention. Circular segment 143 has at least one circle segment 143 having an arc defining at least one tip emission sector emitting electromagnetic radiation 152 at a plane defined by the arc. Tip emission sector may operate upon bending of fiber 110 or independently therefrom.

Embodiments of the invention further comprise optical fibers with an asymmetric cross section, and in particular optical fibers with asymmetric cladding, in which the asymmetry defines at least one emission sector and at least one non-emitting sector in cross section. The difference in the refractive indices between the emitting and non-emitting sectors may be larger than 0.1%, larger than 0.5% or larger than the difference in the refractive indices between the emitting sector and the fiber's core.

The following illustrations depict examples for devices 100 which implement optical fibers 110 exhibiting bend-emission. Each device 100 may be implemented using any embodiment of optical fiber 110 described above according to the device's specifications, under constraints resulting from manufacturing issues but not from conceptual issues.

FIGS. 19A-19C and 19E-19I are high level schematic illustrations of devices 100 using bend emission, according to some embodiments of the invention. FIGS. 19A-19C illustrate a fiber snare device 100, FIG. 19D illustrates an experimental comparison between a fiber snare device and an electro surgical snare regarding the emission profile; FIGS. 19E-19H illustrate fiber hook devices and FIG. 19I illustrates a device with an optical element 144 designed to generate plane emission from the fiber. Optical element 144 may be one or more of operable elements 130.

Device 100 comprises at least one optical fiber 110 and at least one light source 70 (see e.g., FIG. 20C), arranged to transmit electromagnetic radiation through the at least one optical fiber 110. Optical fiber 110 comprises at least one core 103 and cladding 115. Each optical fiber 110 has at least one specified region that is arranged to emit electromagnetic radiation from core 103, upon bending optical fiber 110 at the region(s) beyond a specified bending threshold. Emitting regions 130B may be single or multiple, and may be used to deliver radiation at different intensities and wavelength ranges, possibly for different purposes, such as cutting, ablation, different kinds of treatment and marking.

For example, optical fiber 110 may be formed as a snare (FIGS. 19A, 19B) with an apical bended region arranged to emit electromagnetic radiation 152 in a plane defined by the snare. Device 100 may improve on prior art electrosurgical snares by creating a much more precise and cleaner cut of an obstruction 95, e.g. of polyps, as illustrated in FIG. 19B. The plane of emission 152 is in the inner part of the snare and emission 152 results from the bending of optical fiber 110 at the apical region of the snare. In embodiments, no or very low radiation is emitted outwards, in region 130A. As an applicative example, FIG. 19B illustrates snare-like optical fiber 110 applied through a channel 77 of an endo scope 76.

In embodiments, optical fiber 110 of the snare may have an additional emitting region 130C, e.g. defined by a sector 115C of the cladding at an angle to main emitting sector 115B of the cladding. Region 130C may emit electromagnetic radiation 151 having different characteristics than electromagnetic radiation 152 used for cutting, for example, weaker radiation or radiation in a different wavelength range, that may be useful e.g. for ablating the edges of obstruction 95 to prevent bleeding and prevent infection. It is noted that such ablation is more effective than prior art laser ablation, as radiation 151 is applied smoothly and in the correct direction due to the structure of the snare itself (see FIG. 19B).

A further advantage of fiber snare 110 with respect to a prior art electrosurgical snare is illustrated in experimental results depicted in FIG. 19D, namely the cutting plane of fiber snare 110 is much narrower than the cutting plane of electro surgical snare, causing hence less damage to adjacent tissue and producing a cleaner cut.

FIGS. 19E and 19F illustrate fiber hook device 100 in which optical fiber 110 is bended at an angle and held by a holder 316 or self-sustained (respectively) and the bend is arranged to emit electromagnetic radiation 152 in a plane defined by the predefined bending angle (respectively). Such fiber hook device 100 may operate similarly to the fiber snare under different geometries of obstruction 95. For example, fiber hook may replace a similar electrosurgical hook in treating upper GI lesions by Endoscopic Submucosal Dissection (ESD).

In embodiments of device 100, light source 70 may comprise at least two light sources 70 configured to transmit at least one treatment beam and at least one guiding beam through at least one optical fiber 110. For example, a guiding beam may be used to mark the treatment plane of fiber snare 110 or fiber hook 110 without affecting obstruction 95. Such a guiding beam may be very useful for surgical planning and may further enhance the accuracy of operation using device 100.

FIGS. 19G and 19H illustrate fiber 110 as a J shaped device in which emitted radiation 152 is emitted in a plane from the curved part of the “J”, either outwardly, as illustrated in FIG. 19G, or inwardly, as illustrated in FIG. 1911, selected according to clinical needs and design consideration (a single device 100 may have both functions, e.g., have a different fiber for each purpose, or operate differently at different wavelength regions, or different devices may be designed for these emission patterns). FIG. 1911 further illustrates an embodiment in which the fiber tip is designed to direct energy emitted therefrom sideways, in the plane of radiation, e.g. by designing a reflective element 165 (e.g. a coating or an attached mirror) at the tip which is coupled to circular segment 143 designed to direct emitted tip energy sideways in the emission plane.

FIG. 19I illustrates device 100 with an optical element 144 designed to generate plane emission 152 from fiber 110, according to some embodiments of the invention. For example, optical element 144 may be designed to reflect radiation 152 emitted from the bended emission region 130B of fiber 110 in a designated plane (e.g. as an enhancement of the snare-like device described above).

FIGS. 20A-20C illustrate configurations of controlling the electromagnetic radiation transmitted through fiber 110, according to some embodiments of the invention. With respect to the design of device 100, the transmitted radiation that is not emitted must be controlled and regulated.

In an embodiment illustrated in FIG. 20C, transmitted radiation may go through fiber 110 from source 70 and introductory fiber 166 to exiting fiber 168 and an absorptive sink 169 at the fiber's end (of course introductory fiber 166, treatment fiber 110 and exiting fiber 168 may be implemented as a single fiber, with only fiber 110 exhibiting bend emission in the specified region). Such configuration may also be applied when fibers 110 are weaved around balloon 122 (see below) and then guide the transferred radiation to exiting fiber 168. Absorptive sink 169 may also be located at the end of device 100, as illustrated in FIG. 13C above.

In embodiments illustrated in FIGS. 20A and 20B, a tip 163 of optical fiber 110 is reflective to reflect non-emitted electromagnetic radiation. In FIG. 20A, the reflectance is achieved by a Bragg grating 164 of appropriate characteristics, in FIG. 20B, the reflectance is achieved by a coating 165 (or an attached mirror 165) and exiting fiber 168 is arranged to receive reflected electromagnetic radiation. Using reflective tip 163 not only helps dealing with excessive radiation, but also doubles the potential bending emission, by running the radiation twice through the specified region.

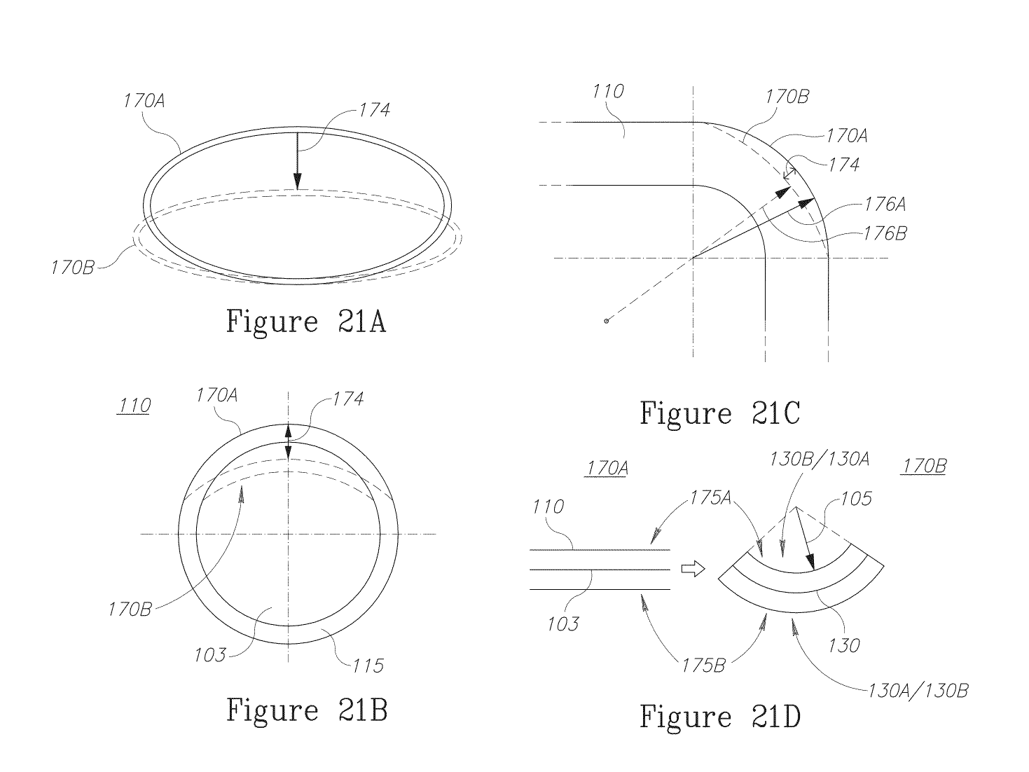

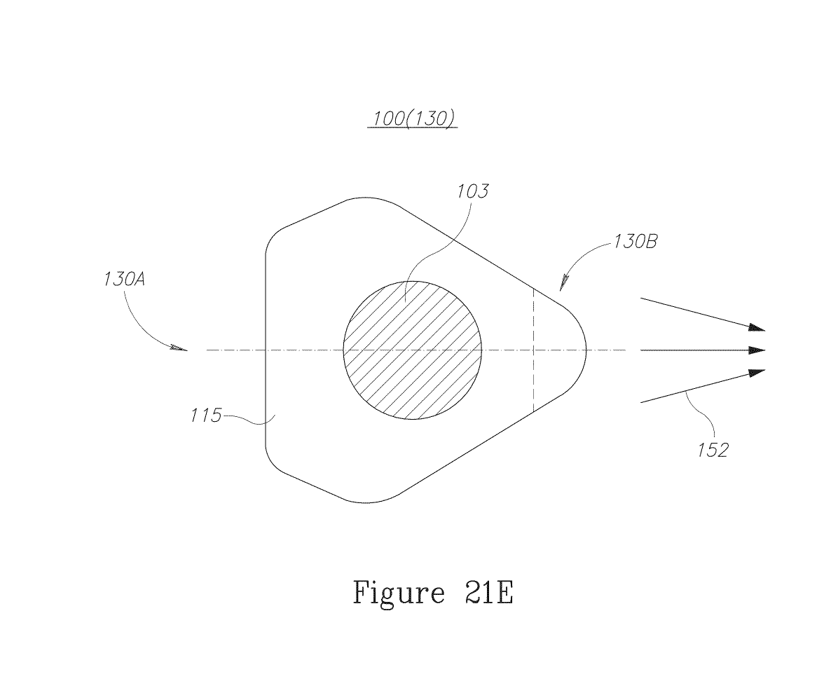

FIG. 21A-21E illustrate cladding 115 of fiber 110, becoming asymmetric in cross section upon application of a strain 174 at the specified region, according to some embodiments of the invention.

Normally, fiber materials like silica tend to have lower values of tensile stress than compressive stress. Basically this means that when bending a symmetrical fiber the breaking point is typically determined by reaching the tensile stress limit. In one embodiment, the effective tensile stress limit is geometrically increased using hollow structures such as tubes. Fiber 110 may be constructed as a hollow tube (FIGS. 21A, 21B), or the hollow tube may be located only on the outer convex rim of bended fiber 110 (e.g., the large air gap 118 in FIG. 17D). The hollow tube may have a circular, oval, or other cross-section so as to increase the natural tendency to collapse inward (FIG. 21A). When fiber 110 is bent, strain 174 increases on the outer rim of fiber 110 (FIG. 21B). Since the center is hollow, an inward collapse is possible. This collapse means a larger bending radius 105 (176A to 176B in FIG. 21C), which in turn means effectively weaker stress, hence the possibility of smaller bend radiuses (sharper bends) for the same stress breakage values. The tube wall thickness and material are accommodated to the possibility of bending. This method allows tighter bending of fiber 110 without breaking it. Another possible embodiment is to use serrations on the outside of the fiber bend region, as in FIG. 1711, which also effectively reduces the stress values on the outer convex rim.

In embodiments, cladding 115 may be arranged to become asymmetric in cross section upon application of strain 174 at the specified region. The asymmetry may be expressed in a form of cladding 115, a thickness of cladding 115 and/or in the refractive index of cladding 115. The asymmetry may be configured to focus emitted electromagnetic radiation 152.

FIGS. 21A-21C illustrate three type of strains 174. FIG. 21A illustrates an asymmetric cross-section of fiber 110, e.g. an elliptic cross-section, which is deformed from state 170A to state 170B with an increased eccentricity. FIG. 21B illustrates circular cross-section fiber 110 that is deformed on one side from state 170A to state 170B, resulting in a change in strain 174. FIG. 21C illustrates bended fiber 110, in which bending strain is decreased by strain 174 while the bending radius of fiber 110, increases from 176A in state 170A to 176B in state 170B.

FIG. 21D illustrates that a bend of fiber 110 from state 170A to state 170B results in different strains in an inner side 175A of fiber 110 and in an outer side 175B of fiber 110, both sides with respect to the bending. Hence different strains are experienced by sides 175A, 175B, which may result in changes in the refractive indices of the two sides, causing bend emission to occur on either side, depending on fiber type and design.

In embodiments, photoelasticity may be used to change the index of refraction under stress, in order to fine-tune the ability of the waveguide (e.g., fiber 110) to discharge energy asymmetrically. The index of refraction of most materials depends on stress. Typically in glass, when stress is increased the index of refraction increases as well. Photoelasticity is caused by the deformation of the electron shells of atoms and molecules and by the orientation of optically anisotropic molecules or components of such molecules; in polymers, it is caused by the uncoiling and orientation of polymer chains. For a small uniaxial tension or compression, Brewster's law is satisfied, such that the optical path equals the index of refraction times the geometrical, or the stress index multiplied by the stress and the geometrical path.

When a waveguide like an optical fiber is bent, a symmetry break occurs in the stress distribution in the fiber's cross-section. Inner side 175A is compressed and outer side 175B expands, resulting in an increase in tensile stress. Solving the Euler-Bernoulli beam equation implies that pure (convex) bending causes zero stress at the neutral axis (FIG. 21D), tensile stress at convex rim 175B, and compressive stress at concave rim 175A; it also implies that the maximum tensile stress occurs at the convex surface and the maximum compressive stress at the concave surface.

The bend causes a symmetry break between cladding 115 on the inside of the bend, the radiation emitting section, and cladding 115 on the outside of the bend. Typically, the cladding's refraction index is lower than that of core 103 by about 0.5%. A bend in the fiber effectively closes the index gap in compressed side 175A and on the other hand increases the gap on outer side 175B. This makes the light confinement weaker on the inner side making it leakier.

The asymmetry due to the bend guides the discharge 152. By bending the waveguide and the subsequent increase in the index of refraction specifically at inner side of the bend 175A, the direction of the discharge 152 is controlled. The index differential at the boundary between core 103 and cladding 115 is tailored in accordance with the desired bend that causes discharge 152.

FIG. 21E illustrates bended fiber 110 in cross section, with different thicknesses of cladding 115 that result in sector 130B focusing the emitted electromagnetic radiation 152. The deformation of cladding 115, and the subsequent index change, may further assist in focusing radiation 152 by emitting sector 130B.

The cross-section of fiber 110 may be designed to control the stress profile. FIG. 21E, for example, illustrates emitting sector 130B as a concave part of cladding 115 that is structured to serve as a converging lens at emitting sector 130B. In this case, compressive stresses are increased in the concave side, which changes the indices of refraction in this area, so that in effect a lens in the concave side of cladding 115 is obtained.

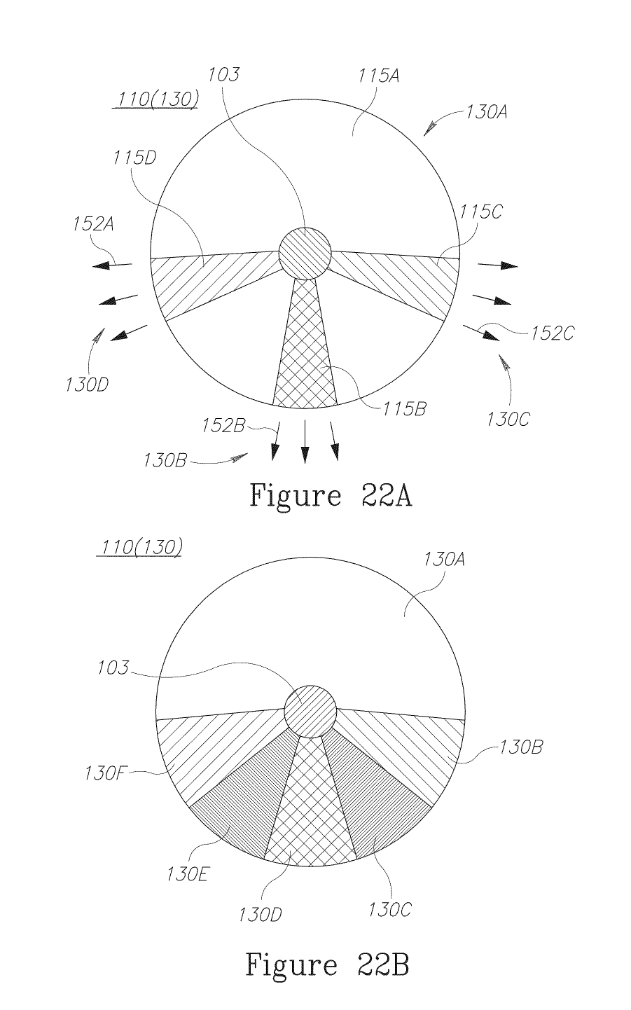

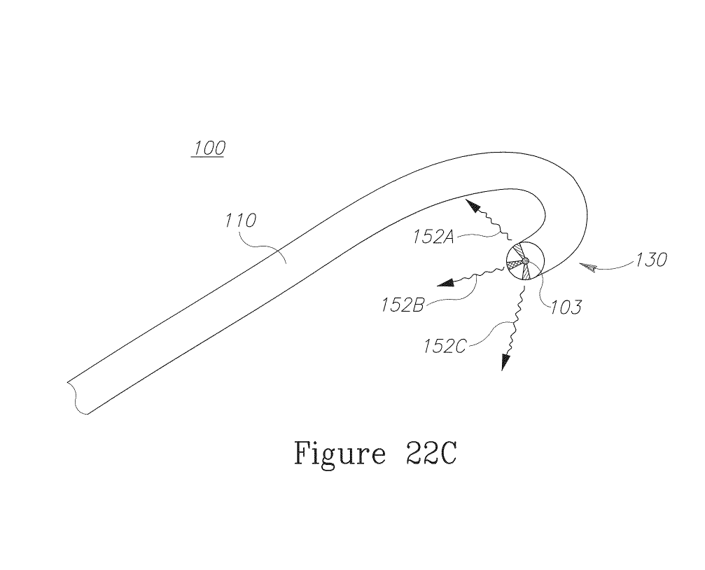

FIGS. 22A and 22C schematically illustrate operable elements 130 on fiber 110 having three emission sectors 130B, 130C and 130D in the emission region and a hook device 100 using this fiber, according to some embodiments of the invention. Emission sectors 130B, 130C and 130D have cladding materials 115B, 115C and 115D respectively, which are different from cladding material 115A in non-emitting sectors 130A and may also be different from each other in the characteristics of emitted radiation 152B, 152C, 152A respectively. For example, central emission sector 130B may be configured to cut tissue, while lateral emission sectors 130C and 130D may be configured to coagulate the sides of the cut by emitting radiation 152C and 152D with specified parameters (e.g., different wavelength ranges, different bending thresholds and different intensities than emitted radiation 152B).

FIG. 22B schematically illustrates operable elements 130 on fiber 110 having multiple emission sectors 130B, 130C, 130D, 130E and 130F in the emission region, according to some embodiments of the invention. The emission sectors may be designed to have different bending thresholds, such that emission intensity from fiber 110 has a specified profile with respect to the circumference of the emission region. For example, central sectors may have the lowest bending thresholds (i.e., the largest threshold bending radii) to emit first and most intense for e.g., a cutting treatment, while peripheral sectors may have higher bending thresholds (i.e., smaller threshold bending radii) to emit last and weaker for less intensive tissue treatment e.g., a coagulation or soldering treatment. In embodiments, the configuration of the emission sectors may be symmetric around central sector 130D.

In addition to providing an emission profile, devices 100 and fibers 110 may be designed to correlate the forces applied thereupon to the intensity of radiation 152, as larger forces produce stronger bending of fibers 110, resulting in more intense radiation. Such designs may enhance the feel provided by device 100 and make the feel more similar to cutting with a regular scalpel mechanically.

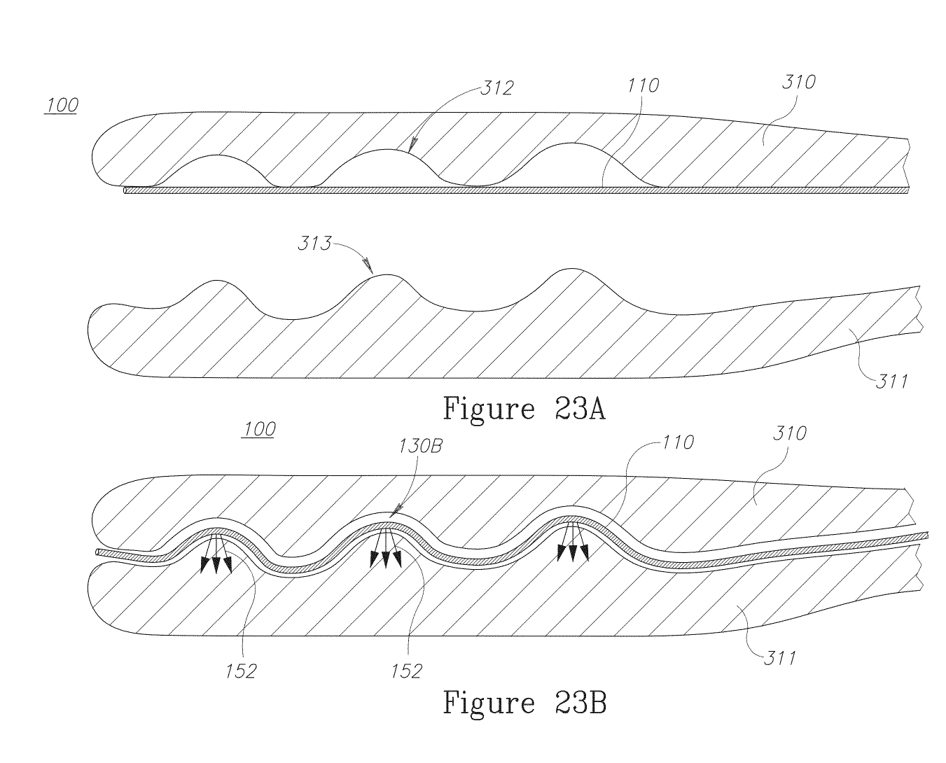

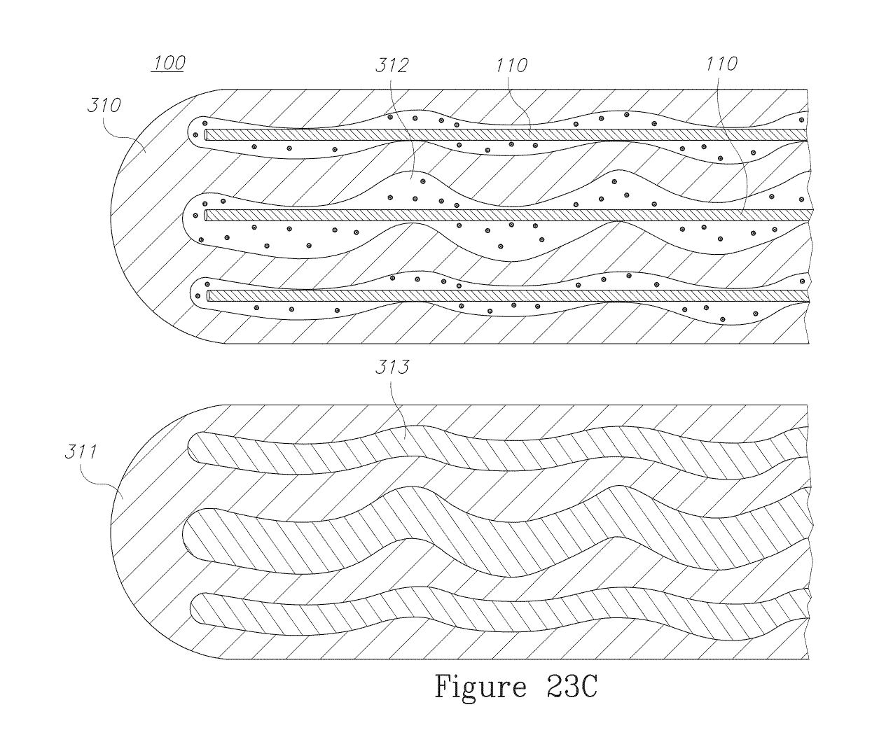

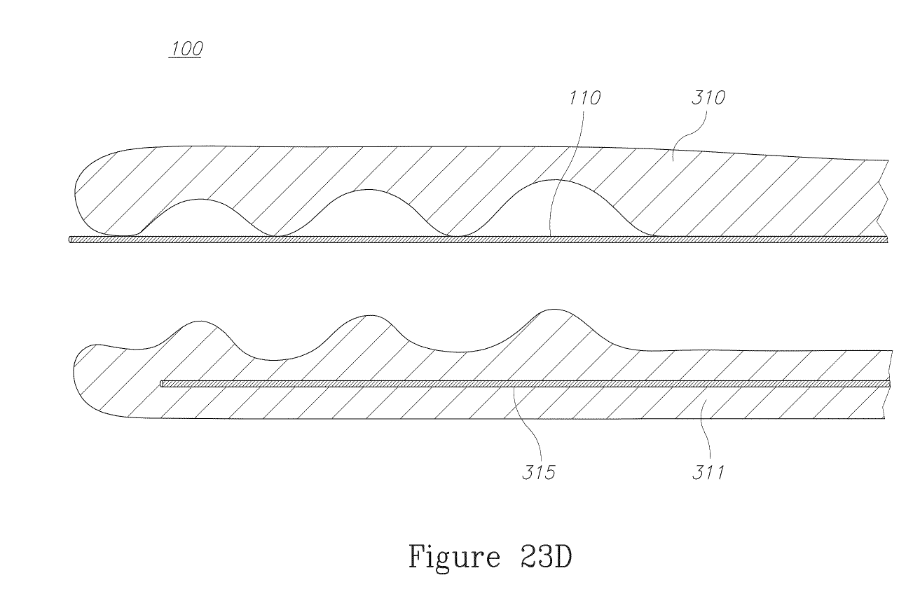

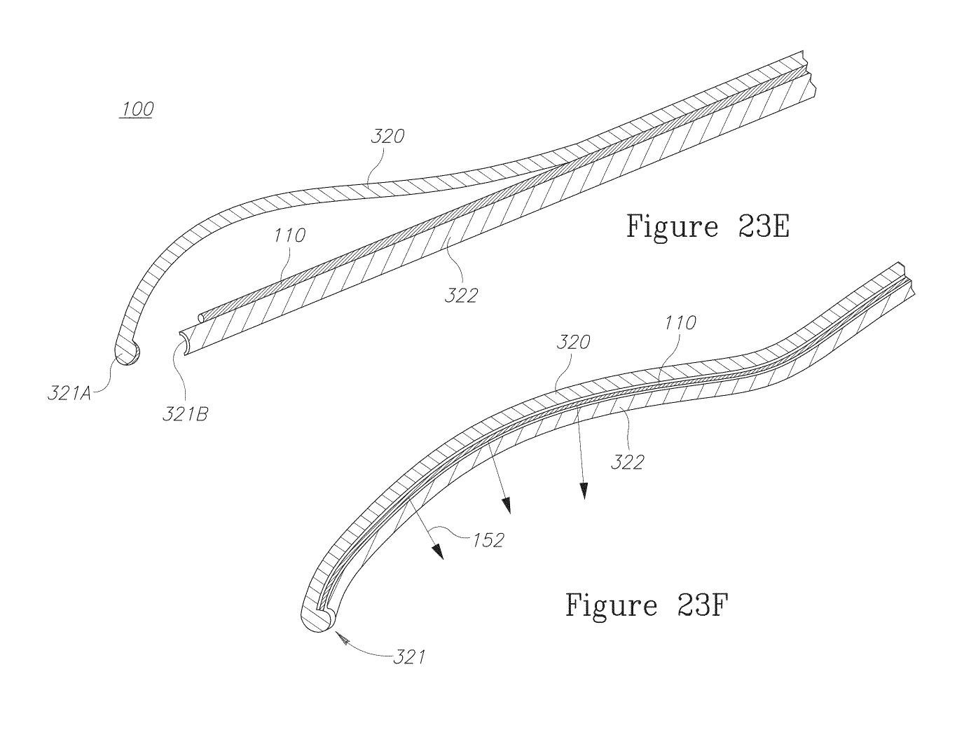

FIGS. 23A-23E schematically illustrate device 100 configured as tweezers, according to some embodiments of the invention. FIGS. 23A and 23B illustrate an open, non-emitting, position and an active position of device 100, respectively. Fiber 110 is integrated within tweezers device 100 in a way that causes bending of fiber 110 upon handling tissue with device 100 to yield radiation emission from operable elements 130 embodied as bended regions 130B which enhances treatment of the handled tissue. For example, fiber 110 may be associated with one arm 310 of tweezers device 100 and fiber bending may occur upon pressing the fiber against a second arm 311 of tweezers device 100. Any of the tweezers' arms may comprise protrusions 313 and/or corresponding recesses 312 to enhance fiber bending upon handling tissue by tweezers 100. Tweezers-like device 100 may comprises surface features designed to control the bending of optical fiber 110 upon tissue contact. FIG. 23C illustrates tweezers device 100 having multiple fibers 110 which may have differing emission characteristics, e.g., configured to apply different effects to the treated tissue.

Tweezers device 100 hence allows mechanical handling while using laser for cutting tissue. The emission may be dependent on the extent of the force applied by the physician through the extent of resulting bending of fiber(s) 110. The closer arms 310, 311 are pressed together, the larger becomes the fiber bending and the emitted radiation.

FIG. 23C schematically illustrates designing the surface area of arms 310, 311, i.e., protrusions 313 and recesses 312, to cause different levels of bending of fibers 110 upon using the tweezers. The surfaces of the arms may be designed to cause different intensities of radiation from fibers 110 (as fibers 110 are bended at different levels) and thus applying different levels of treatment to the handled tissue. Fiber parameters and arm surface parameters may be combined to induce variable treatment levels and effects along and across tweezers device 100. For example, a middle more curvy line in FIG. 23C may lead to more emission from the central fiber 110 and thereby cut tissue, while the lateral less curvy lines may lead only to a soldering effect on the tissue by the lesser bending of lateral fibers 110. FIG. 23D illustrates an embodiment that comprises an additional sensing fiber 315 that may be used to control emitted radiation by fiber 110 by sensing the radiation level emitted therefrom. Some of emitted radiation 152 may enter sensing fiber 315 when the fiber 110 is close to fiber 315 due to the closing of tweezers device 100 and when no tissue obstructs the radiation path. The intensity of the entering radiation may be used to indicate treatment efficiency and provide information on the handled tissue. Any other sensor may be used in place or in addition to additional fiber 315, e.g., a temperature or an impedance sensor.

FIGS. 23E and 23F illustrate an open, non-emitting, position and an active position of device 100, respectively. Fiber 110 is integrated within tweezers device 100 in a way that causes bending of fiber 110 upon handling tissue with device 100 and radiation emission from the bended regions which enhances treatment of the handled tissue. For example, fiber 110 may be associated with one arm 310 of tweezers device 100 and fiber bending may occur upon pressing the fiber against a second arm 311 of tweezers device 100. In the illustrated embodiment, tweezers-like device 100 comprises one arm 320 that is curved and stiff and a second arm 322 which is flexible to which fiber 110 is attached. Arms 320, 322 may comprise reciprocally engaging tips 321A, 321B respectively that engage upon full closure of device 100. Fiber 110 may be arranged to emit at the resulting bending of flexible second arm 322. Multiple fiber embodiments may be designed to provide varying tissue handling effects.

In embodiments, fiber 110 may reach tip 321A of arm 322 and be used additionally as a tip-emitting fiber (“straight shot laser”).

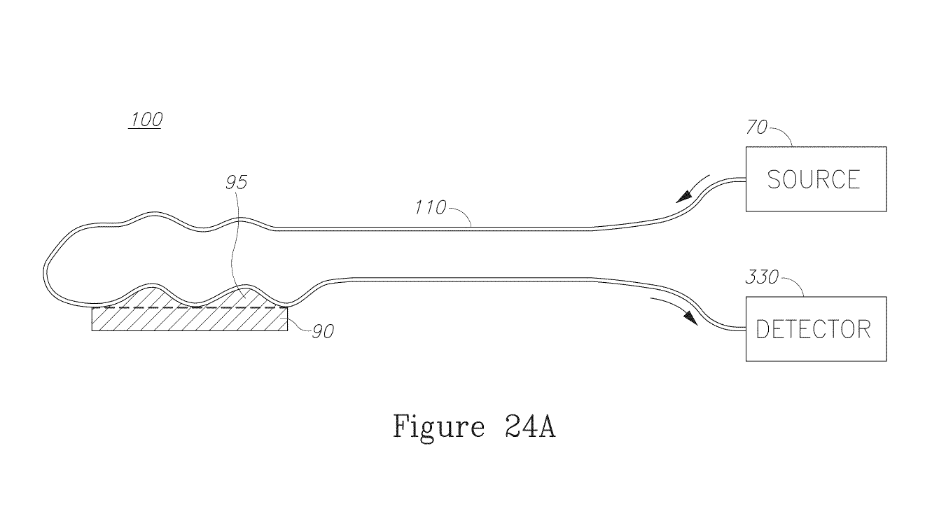

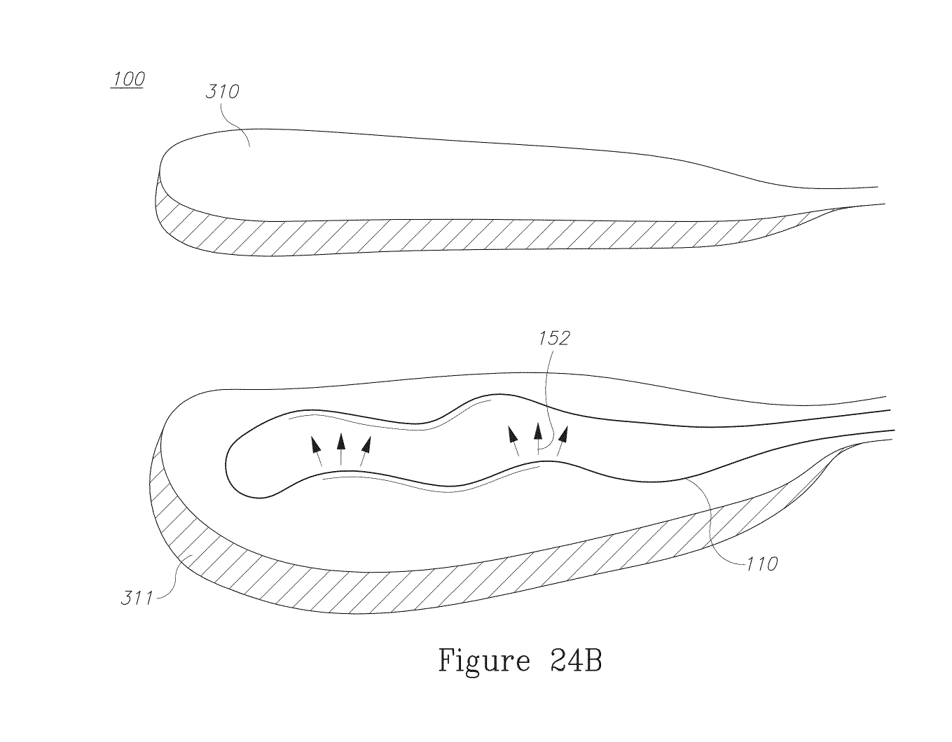

FIGS. 24A and 24B schematically illustrate device 100 with a feedback loop for controlling emitted radiation 152, according to some embodiments of the invention. For example, the feedback loop may be used for sensing data from additional sensing fiber 315 or other sensor illustrated in FIG. 23D. FIG. 24A illustrates radiation source 70 and a radiation detector 330 which measures radiation after emitted radiation 152 was emitted along the fiber, to determine the extent of radiation emission 152 and hence of treatment. Radiation detector may be replaced or enhanced by a temperature sensor. FIG. 24B illustrates tweezers device 100 with fiber 110's emitting regions, from which radiation is emitted. Data from detector 330 may be used to control radiation parameters such as activation, wavelength range and intensity as well as parameters of device 100—e.g., provide feedback to the treating physician, change device 100's electronic parameters etc. Detector 330 may measure changes in bend-emitted radiation 152 via the natural changes that occur to the tissues' index of refraction as it is being coagulated or cut.

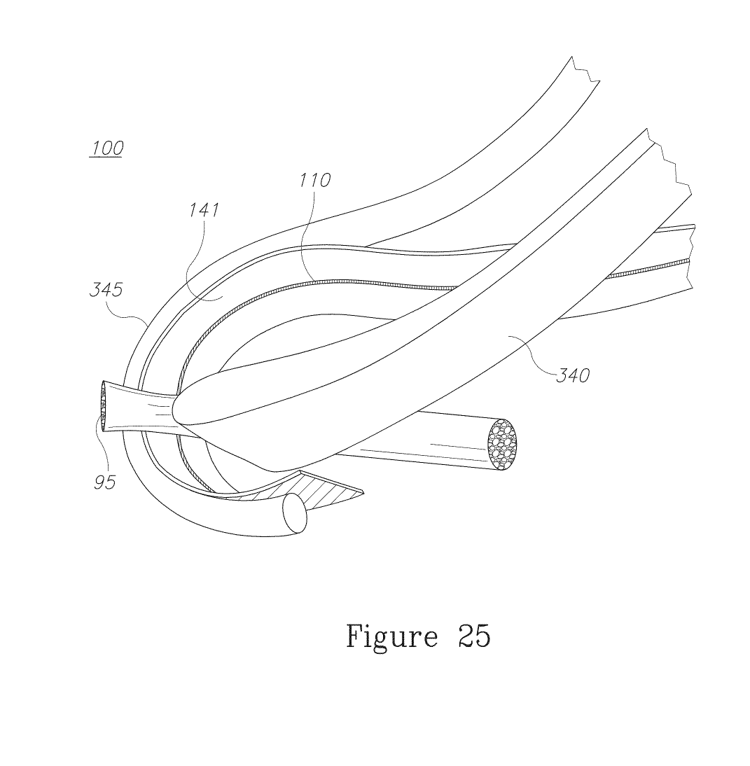

FIG. 25 schematically illustrates device 100 for cutting vessels, according to some embodiments of the invention. Device 100 comprises a fixating arm 340 (e.g., a hook) and a cutting arm that comprises fiber 110 that is optionally supported by supporting structure 141 and a second fixating arm 345 that stabilizes target 95 (such as a blood vessel) and fiber 110's contact thereto. The cutting arm may comprise a single fiber 110, e.g. fiber 110 with multiple emission sectors as illustrated in FIGS. 22A, 22B, or multiple fibers 110 with similar or varying characteristics, optionally attached to supporting structure 141. In embodiments, fixating arms 340 and 345 may be part of the supportive structure, as a variation of tweezers-like device 100. Upon pressing fixating arm 340 against the cutting arm (supported by second fixating arm 345) around target 95 such as a blood vessel, cutting of vessel 95 and immediate soldering of the cut ends of vessel 95 are carried out, e.g. by activation of different treatment effects as described above. Alternatively, the cutting arm with second fixating arm 345 may press vessel 95 against fixating arm 340 and either arm may emit radiation to apply the treatment upon vessel 95.

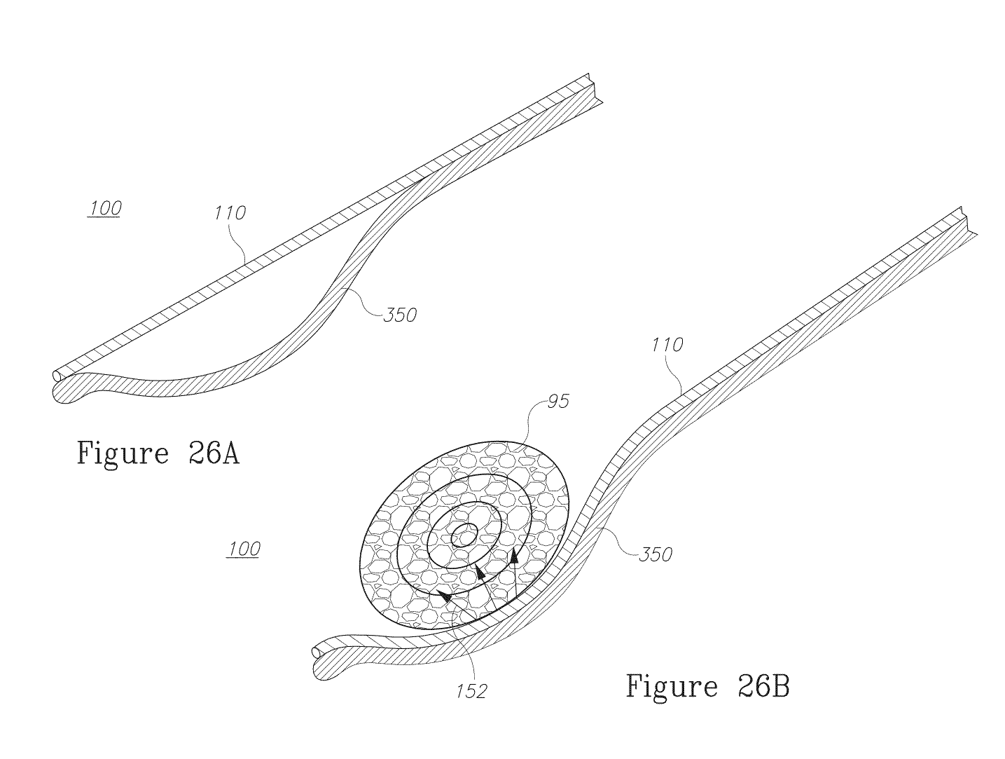

FIGS. 26A and 26B illustrate device 100 for treating target 95 by pressing device 100 upon it, according to some embodiments of the invention. Device 100 may be mechanically configured similarly to tweezers device 100 illustrates in FIGS. 23E and 23F, and emit radiation externally, onto target 95 against which device 100 is pressed. Device 100 may have a single arm 350 that supports fiber 110 and allows bending of fiber 110 upon contact with target 95, in accordance with the applied pressure and in correspondence to the form of target 95. For example, fiber 110 may be loosely or movably attached to the tip of arm 350, fiber 110 may be flexible or device 100 may be designed to provide for some lengthening of fiber 110 upon contact with target 95.

The illustrated tweezers with arms 310 and 311, 320 and 322, 340 and 345 may be embodiments of supportive structure 141 described above.

Embodiments of the invention provide device 100 as a vessel sealing tip 100 for surgical forceps which allows, via operable elements 130 implemented e.g., in fiber 110, both sealing a vessel section and cutting therethrough without extracting the tip out of the body or exchanging the tip. Either a single action yields the sealing and the cutting, or two or more tip actions may be carried out sequentially to perform the sealing and cutting operations. In addition, the tip may be used for cutting through tissue. Embodiments of the tip may utilize any energy source via operable elements 130, in particular optical laser energy but also RF or ultrasound energy. The different effects (sealing, cutting) may be achieved by varying the emitted energy spatially, by manipulating the vessel prior or during energy delivery, by changing a configuration of the tip during operation and by combining tensile forces or ablation at appropriate locations of the vessel.

FIGS. 27A-27C are high level schematic illustrations of a vessel sealing tip 100 for surgical forceps 92 according to some embodiments of the invention.

Vessel sealing tip 100 may comprise an energy delivery element 110 such as at least one optical element 110 arranged to deliver, upon actuation, electromagnetic radiation 152 to a vessel 90 to cut vessel 90 at a cutting region 96B (FIG. 27B) or to yield a vessel welding effect in a specified sealing section 96A of vessel 90 (FIG. 27C), and to cut vessel 90 at a cutting location 96B within specified sealing section 96A. For example, at least one optical element 110 may comprise at least one optical fiber 110 arranged to deliver electromagnetic radiation such as laser energy. In case of cutting region 96B, radiated energy 152 may also seal edges of the cut vessel during the cutting.

Energy delivery element 110 may be attached to any one of two jaws 101 (101A, 101B) of forceps tip 100, or may also be a free element, at least on a part of the length thereof (see below).

In cases of energy delivery element 110 being an optical fiber, fiber 110 may emit radiation 152 (FIG. 27B) that yields a vessel sealing effect and radiation 152 that yields a vessel cutting effect. Radiation characteristics may be temporally varied in a controlled manner or may be designed in advance with respect to one or more vessel types. Radiation 152 may further be used to ablate the vessel wall prior to sealing and/or cutting vessel 110. Radiation 152 may further be used to cut tissue during the advancement of tip 100; for example, fiber 110 may continue beyond the illustrated extension to the very tip of either jaw 101, to their external sides or may extend beyond tip 100 itself (e.g., form a loop ahead of tip 100).

Fiber cross section may be designed according to the principles outlined in FIG. 22A, 22B, in which fiber 110 may be arranged to emit at least two radiation types 152A, 152B at at least two corresponding zones (130A, 130B, FIG. 27C) of fiber 110. The radiation types may differ in at least one of their intensity, spectral range, spatial and/or temporal patterns. Emission zones 130A, 130B may comprise, in cross section, different corresponding fiber sectors 115A, 115B made of different cladding materials or having different refractive indices. In embodiments, emission zones 130A, 130B may have different cross-sections and thus different spatial energy density profiles. In certain embodiments, fiber 110 may comprise a solid core optical fiber (having core 103), a hollow fiber or a photonic crystal fiber (such as a holey fiber, a Bragg fiber or any other micro-structured fiber). In certain embodiments, fiber 110 may comprise a metallic waveguide. The different fiber sections may be differently micro-structured or have a different spatial arrangement of core 103 and cladding (e.g., core 103 may be asymmetrically positioned within the cladding). Fiber 110 may be single-mode or multi-mode. Beam polarization may also be used to differentiate radiation types 152A, 152B and control the emitted energy density spatial distribution.

In certain embodiments, at least one jaw 101 of the forceps may comprise at least one protrusion 94A (FIG. 27C) arranged to constrict vessel 90 prior to the actuation of energy delivery element 110 (such as at least one optical element 110, a RF source, an ultrasound source etc.). Protrusion 94A protrudes from a surface 94B of jaw 101 and constricts vessel 90 at the region of energy deliver to reduce the local thickness of vessel 90 and to provide more spatial variability in possible energy delivery directions. Energy delivery element 110 may be positioned fully or partially within protrusion 94A; for example, at least one optical element 110 may be set within at least one protrusion 94A.

Certain embodiments of the invention comprise a tip 100 with at least two jaws 101 for surgical forceps 92. At least one of jaws 101 may comprise at least one protrusion 94A positioned to contact tissue held by tip 100 and deliver both pressure and external energy to the tissue. The pressure may be a tip holding force (the force applied to the forceps and thereby transferred to the tip's jaws), concentrated by at least one protrusion 94A. The external energy may be any of electromagnetic (e.g., optical, RF), electrical and ultrasound energy, or a combination thereof. At least one protrusion 94A may comprise one or more thin element that concentrates applied forces onto a small section of vessel 90. At least one protrusion 94A may comprise an abrasive or an ablative element that reduces vessel wall thickness or even cuts the vessel, in addition to constricting the vessel.

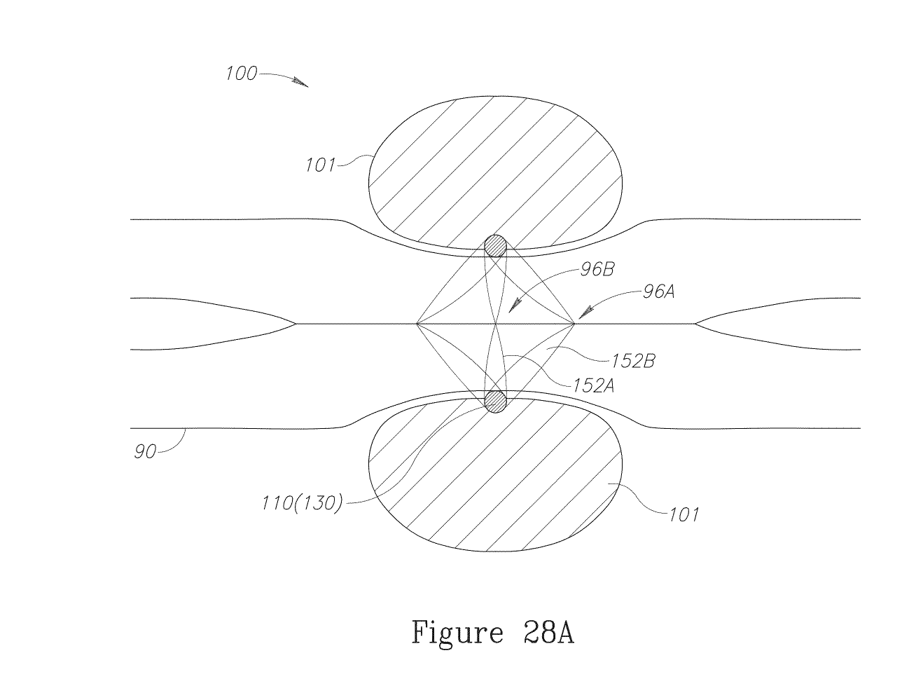

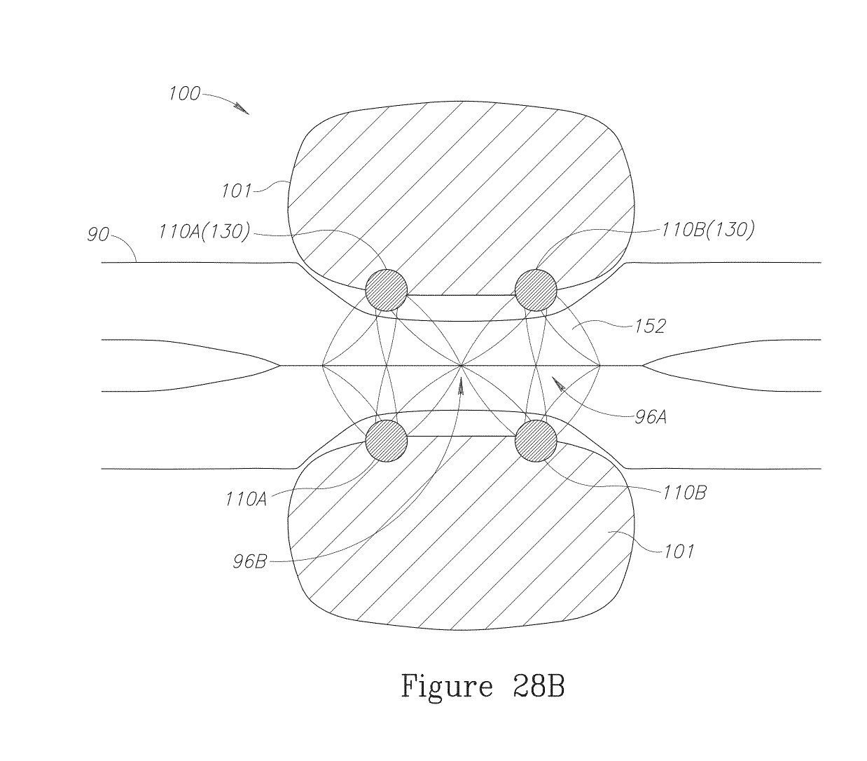

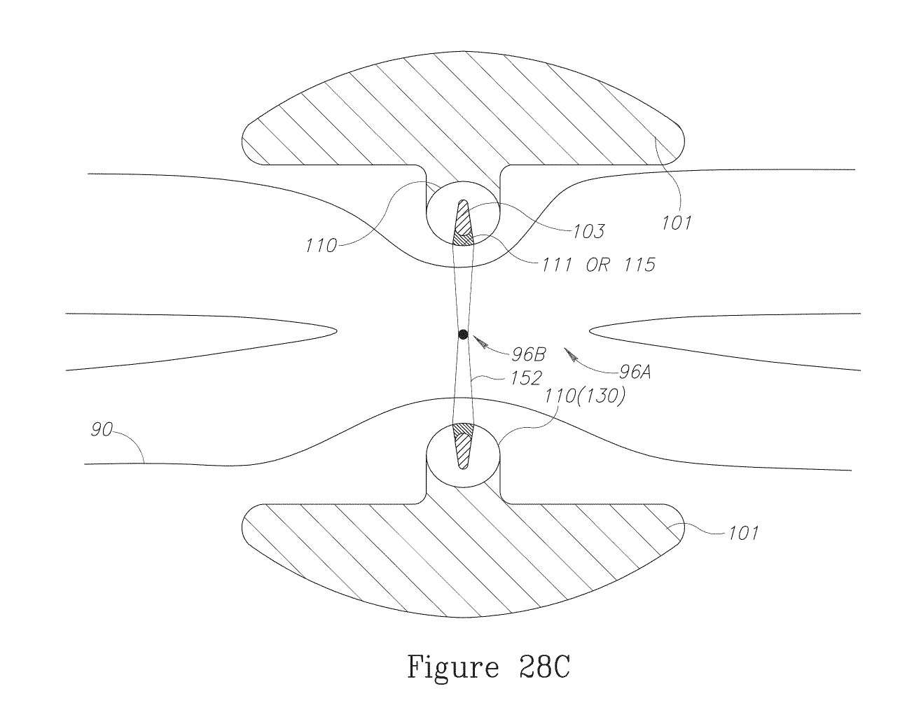

FIGS. 28A-28C are high level schematic illustrations of vessel sealing tip 100 for surgical forceps 92 having focusing elements 111, according to some embodiments of the invention. Energy delivery elements 110 may comprise focusing elements 111 (FIG. 28C) arranged to focus any type of delivered energy (e.g., optical energy, RF, ultrasound, electrical energy, etc.). For example, optical elements 110 may comprise at least one focusing element 111, such as a lens 111 or a sector 115 of the cladding arranged to focus emitted radiation 152. In the non-limiting example illustrated in FIG. 28C, a combination of asymmetric core 103 and focusing element 111 may be arranged to yield the welding and/or cutting of vessel 90 (depending on the delivered radiation and tip manipulation). Vessel 90 may be seen in the context of the forceps devices as obstruction 95 to which energy is delivered and/or as a vessel occluded by obstruction 95. The focusing of the emitted radiation may be in a cross-sectional plane of optical element 110. In certain embodiments, focusing elements 111 may focus different types of radiation 152A, 152B at different regions of vessel 90, e.g., radiation 152B may be focused to produce a sealing effect at sealing region 96A, and radiation 152A may be focused to cut vessel 90 at cutting region 96B. Focusing elements 111 may be embedded in or attached to forceps jaws 101. Focusing elements 111 may be multiply associated with at least one of jaws 101, as illustrated in FIG. 28B. The focusing elements may be embedded in multiple fibers 110A, 110B and be arranged to jointly apply the sealing and cutting to regions 96A, 96B of vessel 90 respectively. Particularly, at least two focusing elements 111 may be positioned on each jaw 101 and arranged to yield a specified extension of specified sealing section 96A, which is broader than sealing section 96A produced by a single focusing element 111 or solely by optical element 110.

In certain embodiments, energy delivery element 110 may be arranged to reduce a vessel wall thickness prior to the welding. For example, optical element 110 may operate in an ablative mode to reduce vessel wall thickness prior of holding vessel 90 sealing it and cutting through vessel 90. The reduction of wall thickness allows energy to be delivered to the internal walls of vessel 90 without causing thermal damage to the external wall of vessel 90. Furthermore, reducing the wall thickness may reduce the wall resistance to mechanical pressure and thus allow a more effective application of pressure to vessel 90, e.g., by protrusions 94A (FIG. 27C), to create a more effective gripping of vessel 90 by forceps 92 and a better sealing effect.

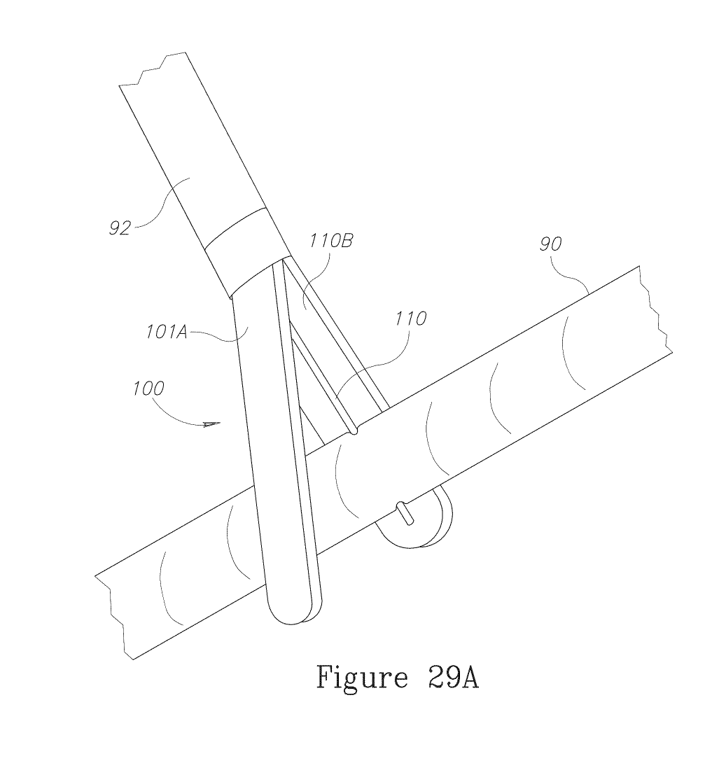

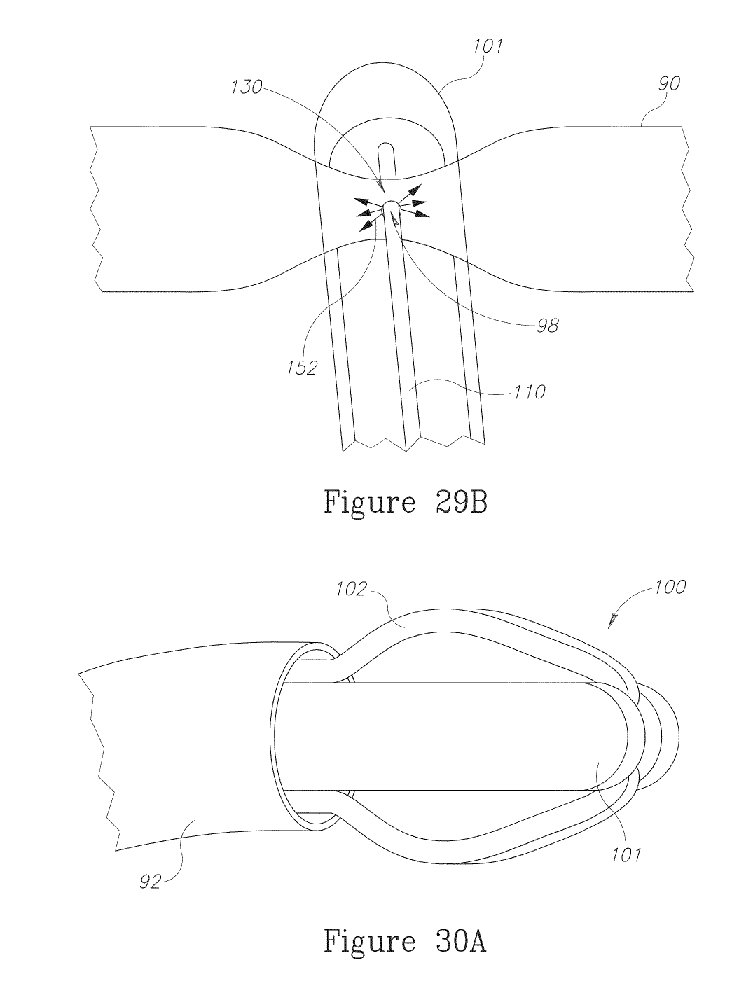

FIGS. 29A and 29B are high level schematic illustrations of vessel sealing tip 100 for surgical forceps 92 having vessel piercing elements 110, according to some embodiments of the invention. Energy delivery element 130 may comprise at least one optical element 110 comprising at least one optical fiber 110 arranged to penetrate a lumen of vessel 90, piercing thereby a hole 98 in vessel 90, prior to the actuation thereof and emission of energy 152 from fiber 110. In certain embodiments, penetrating the vessel lumen enables more efficient sealing and/or cutting of vessel 90. Delivering energy from the interior of vessel 90 allows treating its inner layers directly, without having to apply high pressure on the vessel wall in order to flatten vessel 90. In certain embodiments, penetrating and flattening may be applied simultaneously or sequentially to reciprocally enhance the sealing effect.

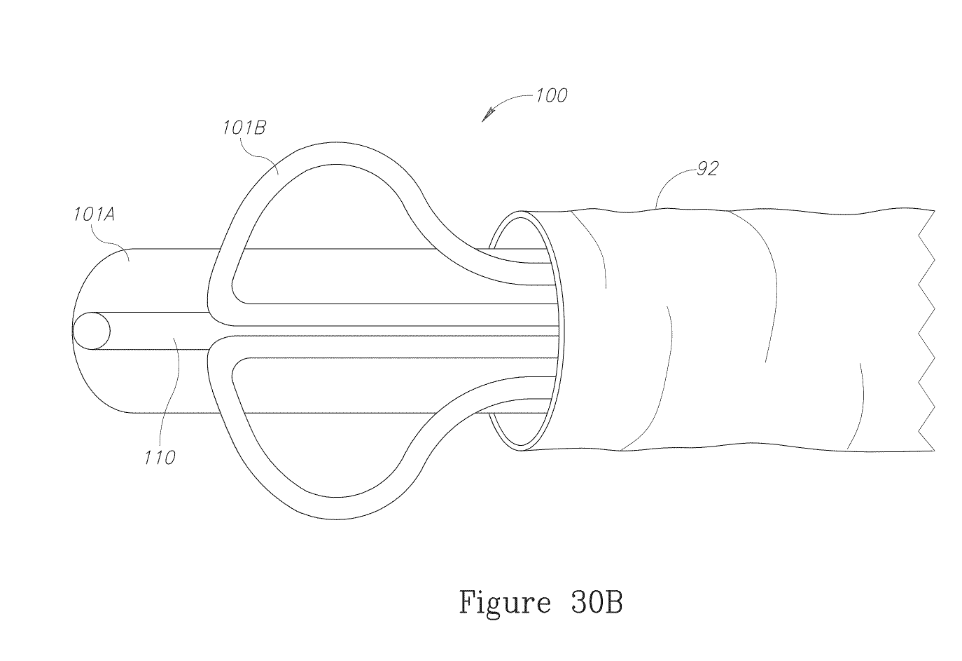

FIGS. 30A and 30B are high level schematic illustrations of vessel sealing tip 100 for surgical forceps 92 having transversely expanding elements 102, according to some embodiments of the invention. At least one jaw 101 of tip 100 may comprise transversely expandable element 102 arranged to yield a specified extension of specified sealing section 96A. Mechanically pressing a wider section of vessel 90 increases the potential sealing section of vessel 90 and hence may improve sealing and vessel manipulation conditions. Transversely expanding elements 102 may be retracted in a tool delivery channel and expand only in situ, upon using tip 100. In certain embodiments, transversely expanding elements 102 are controllably expandable, e.g., by a user of forceps 92 or responsive to applied pressure on respective jaw 101 or sensed resistance of vessel 90. In certain embodiments, transversely expanding elements 102 may mechanically extend specified sealing section 96 during the welding. The welding (e.g., by radiation 152B aimed at sealing section 96) may be combined with transverse forces applied to vessel 90 at the sealing section and aimed to expand the treated section. A reduced resistance of vessel 90 due to the energy radiation and/or the mechanical extending may thus be exploited to expand the sealing section. In certain embodiments (FIG. 30B), at least one of jaws 101 may be designed as a transversely expanding element. For example, one jaw 101A may be a regular jaw and the other jaw 101B may be expandable to broaden the sealing region of vessel 90. Expandable jaw 101B may be made of two or more parts and tip 100 may comprise means to separate the parts to further enhance the stretching effect on vessel 90.

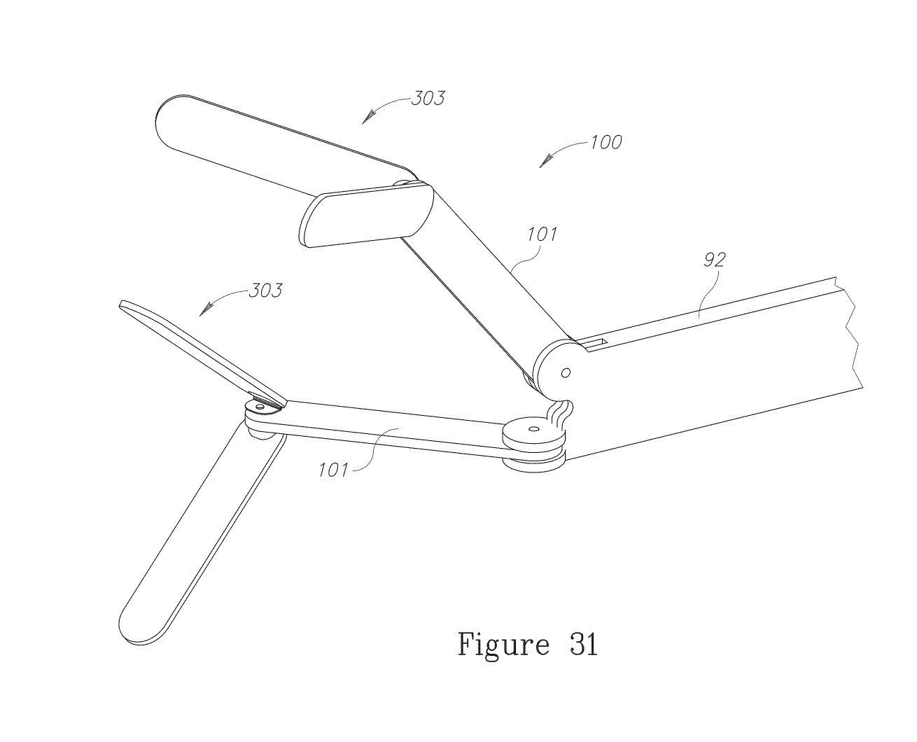

FIG. 31 is a high level schematic illustration of vessel sealing tip 100 for surgical forceps 92 enabling extension of the vessel sealing region, according to some embodiments of the invention. In certain embodiments, one or both jaws 101 may be hingedly attached to forceps 92 and may be controllably pivotally movable to stretch vessel 90, e.g., during sealing or cutting thereof. One or both jaws 101 may comprise a forceps element 303 arranged to hold and/or pull a respective vessel section to generate the stretching effect of the vessel, which expands the sealing region. Jaws 101 may be moveable along different spatial directions, to yield an additional twist of vessel 90, selected to further enhance the stretching of the sealing region. Any of the above mentioned movements and actions may be combined with energy delivery to enhance the sealing and/or cutting effect. Accordingly, control of any of these movements may be carried out by a user or responsive to sensed forces in tip 100. Jaws 101 may by controlled by mechanical compliance to exerted forces.

In certain embodiments, vessel sealing tip 100 for surgical forceps 92 may comprise at least one transversely expandable element 102 or 303 arranged to yield a specified extension of a specified section of vessel 90 and energy delivery element 110 arranged to deliver external energy, upon actuation, to vessel 90 to yield a vessel welding effect in a specified sealing section of vessel 90 and to cut vessel 90 within the specified sealing section. The external energy may be at least one of optical, electrical and ultrasound energy. Tip 100 may thus open up and create a seal larger than half width of tool (e.g., tip 100) or just separate regions of cut and seal. The specified sealing section may be mechanically extended during the welding with or without additional energy delivery. In embodiments, tip 100 may comprise two transversely expandable elements 103, each arranged to yield a specified extension of the specified section of vessel 90 in a different plane.

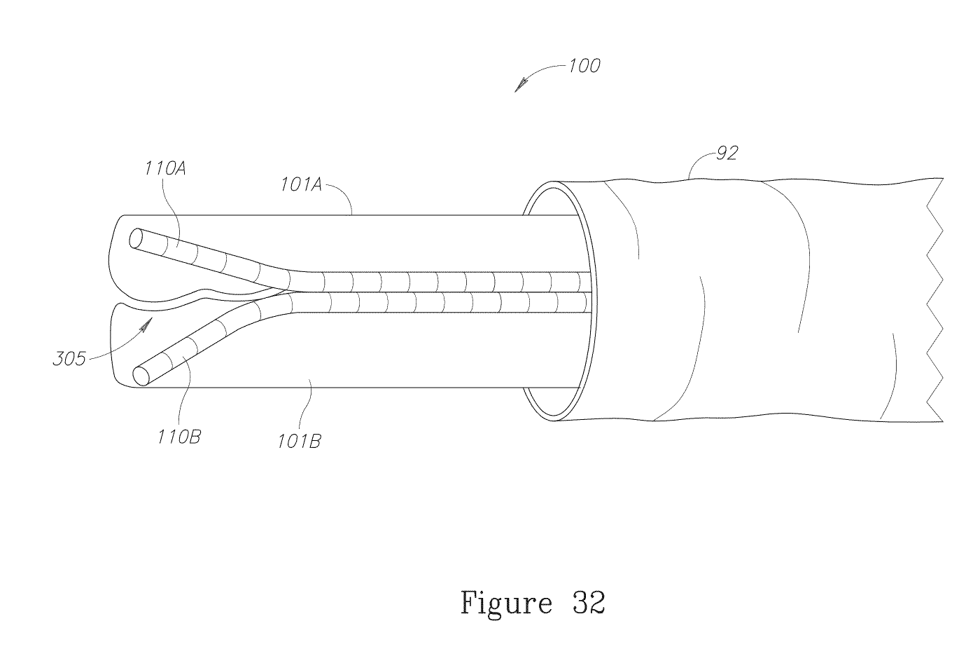

FIG. 32 is a high level schematic illustration of vessel sealing tip 100 for surgical forceps 92 with variable intensity treatment, according to some embodiments of the invention. In certain embodiments, each jaw 101A, 101B of the forceps may comprise at least one optical fiber 110A, 110B respectively, positioned at a distance from an edge 305 of the respective jaw, wherein the distance may vary along the jaws. For example, the varying distance with respect to treating edge 305 of the jaws may diminish from a tip to a base of jaws 101A, 101B to yield the welding effect at the jaw tip and perform the cutting between the tip and the base of the jaw. In certain embodiments, the sealing and cutting effects are thus spatially differentiated along the jaws instead or in addition to the spatial differentiation across the jaws illustrated previously (cf., e.g., FIG. 28A). In certain embodiments, one or more fibers 110A, 110B may have longitudinally varying characteristics that generate radiation 152 of different characteristics along the fiber. For example, radiation 152 designed for sealing may be applied at the jaw tips where the distance to vessel 90 is also the largest, and radiation 152 designed for cutting may be applied at the jaw bases where the distance to vessel 90 is also the smallest. Hence jaws 101 and energy delivery elements 110 may be designed to jointly differentiate welding and cutting effects.

In certain embodiments, vessel sealing tip 100 may be constructed from non-metallic materials to allow use of tip 100 simultaneously with MRI imaging. For example, tip 100 may be made of plastic and energy may be delivered via optical fibers.

In certain embodiments, vessel sealing tip 100 may comprise at least one wave guide (not shown) arranged to deliver, upon actuation, electromagnetic radiation to the vessel to yield a vessel welding effect in a specified sealing section of the vessel and to cut the vessel within the specified sealing section. In certain embodiments, at least one jaw of the forceps may comprise at least one protrusion arranged to constrict the vessel prior to the actuation of the at least one wave guide.

In certain embodiments, vessel sealing tip 100 for surgical forceps 92 may be configured to be applied for any of the following treatments: Sealing blood vessels, arteries, veins; Sealing biliary ducts; Sealing urinary tract; Sealing reproductive tract; Sealing airways; Sealing in the GI tract; Sealing the dura; Treating septums (nasal, atrial, etc.); Sealing organs such as lung, liver, spleen, heart, stomach, pancreas, uterus, bladder, kidney etc. While the above description mainly referred to treating vessels 90, tip 100 for surgical forceps 92 may be configured for treating any other type of tissue, as well as to carry out further surgical tasks, such as cutting or ablating tissue.

In a non-limiting example, vessel sealing tip 100 may be configured to apply pressures in the at least a part of the range 20-400 PSI. The outer diameter of fiber(s) 110 may be between 0.05-2 mm and fiber(s) 110 may be arranged to deliver power levels between e.g. 1 W-100 W. Tip 100 may be configured to have a jaw 101 length between 2-50 mm, a jaw 101 width between 0.5-10 mm, and a ridge width of at least one protrusion 95A between 0.1-5 mm. The dimensions of jaws 101 may be configured with respect to the specific use of tip 100, as illustrated in the examples above. For example, larger tips 100 may be designed to seal larger or stiffer vessels 90.

Table 1 is a non-limiting exemplary overview of possible tip characteristics for various applications of tip 100.

TABLE 1
Tip parameters for various applications

| Vessel type (Sealing | Anatomical | Jaw | Jaw | Working |
|---|---|---|---|---|
| operation, unless | size | length | width | area length |
| otherwise indicated) | (mm) | (mm) | (mm) | (mm) |
| Blood vessels, arteries, | <1 to 10 | 20 and up | 2 to 10 | 17 and up |
| veins | | | | |
| Extremely large blood | up to 25 | 40 to 60 | 2 to 10 | 35 and up |
| vessel, aorta, aneu- | | | | |
| risms, etc. | | | | |
| Biliary ducts | 5-10 | 20 and up | 2 to 10 | 8 and up |
| Urinary tract | up to 10 | 20 and up | 2 to 10 | 17 and up |
| Reproductive tract | Fallopian | 20 and up | 2 to 10 | 17 and up |
| | tubes up to | | | |
| | 2, general | | | |
| | tissue much | | | |
| | more | | | |
| Airways | | 20 and up | 2 to 10 | 17 and up |
| GI tract | | 30 and up | 2 to 10 | 25 and up |
| Dura | | 20 and up | 2 to 10 | 15 and up |
| Septum (nasal, atrial, | | 3 and up | 1 to 10 | 2 and up |
| etc.) | | | | |
| Operating on organs | | 20 and up | 2 to 10 | 17-70 |
| such as lung, liver, | | | | |
| spleen, heart, stomach, | | | | |
| pancreas, uterus, | | | | |
| bladder, kidney, etc. | | | | |
| Neurological opera- | | 3 and up | 2 to 10 | 3 and up |
| tions | | | | |
| Kidney operations | | 20 and up | 2 to 10 | 17 and up |

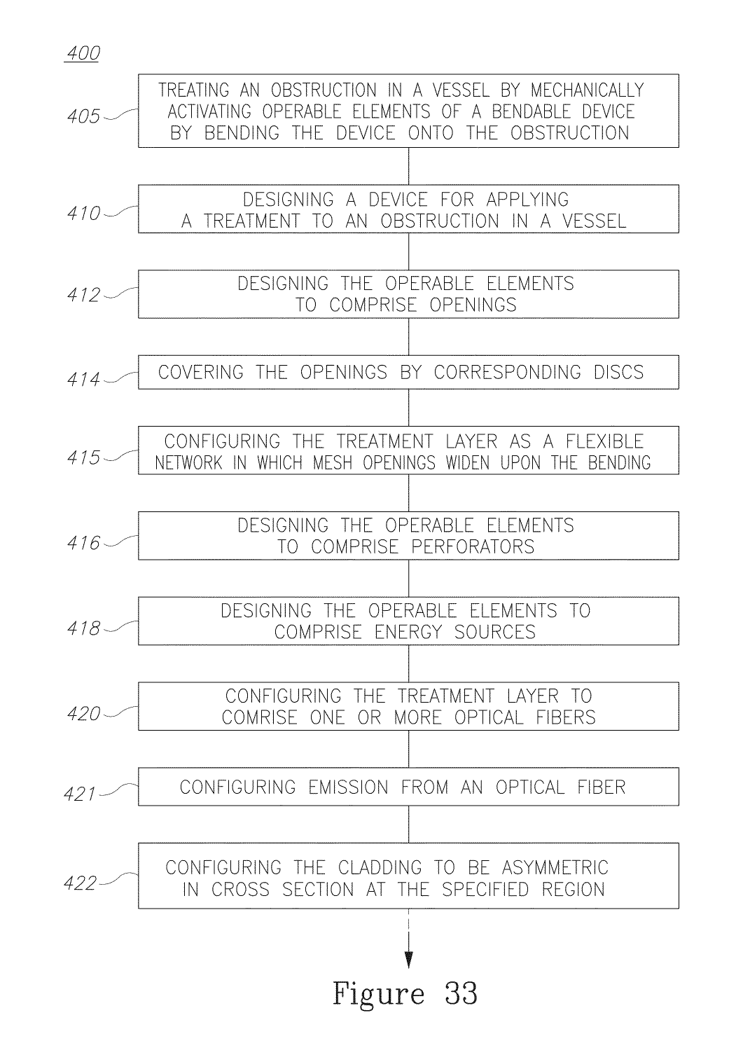

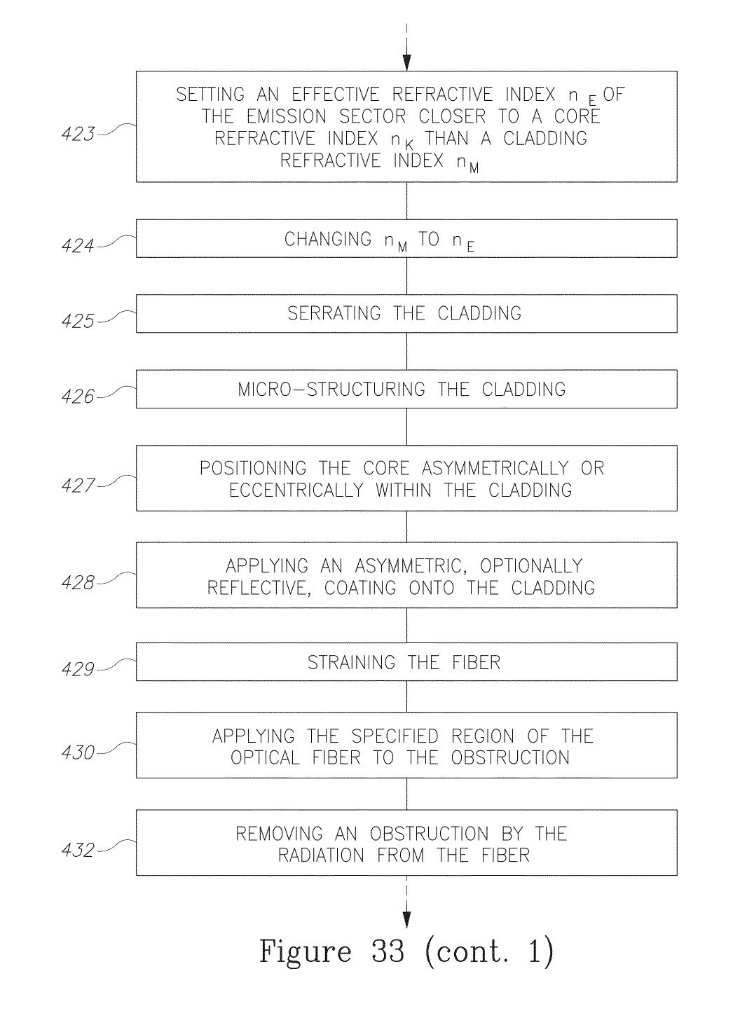

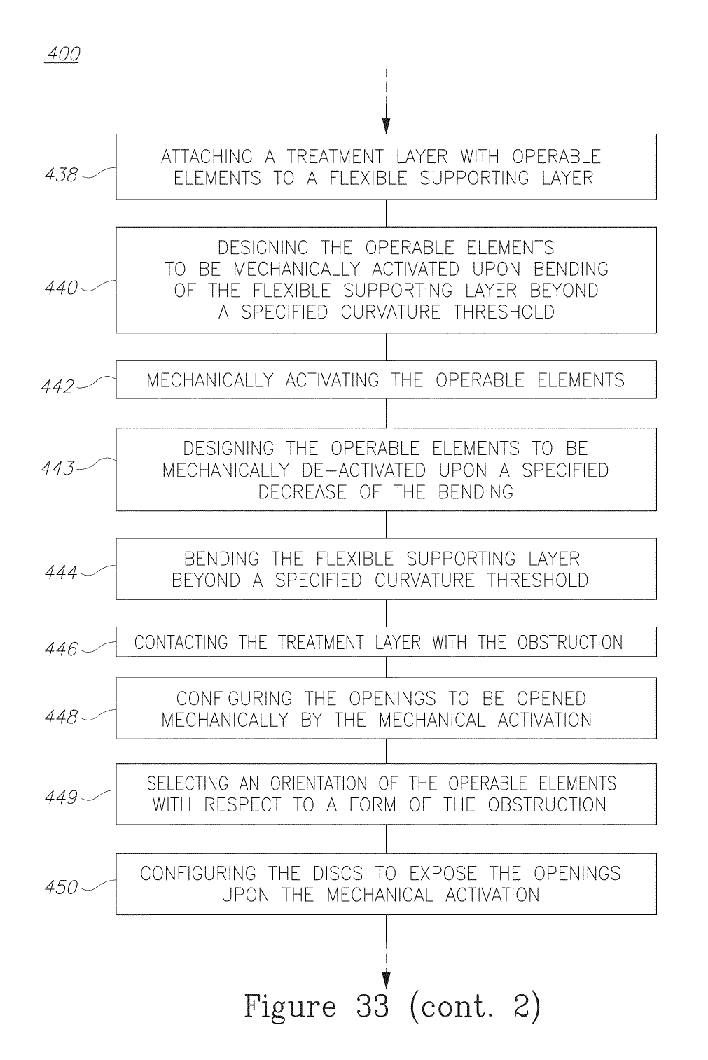

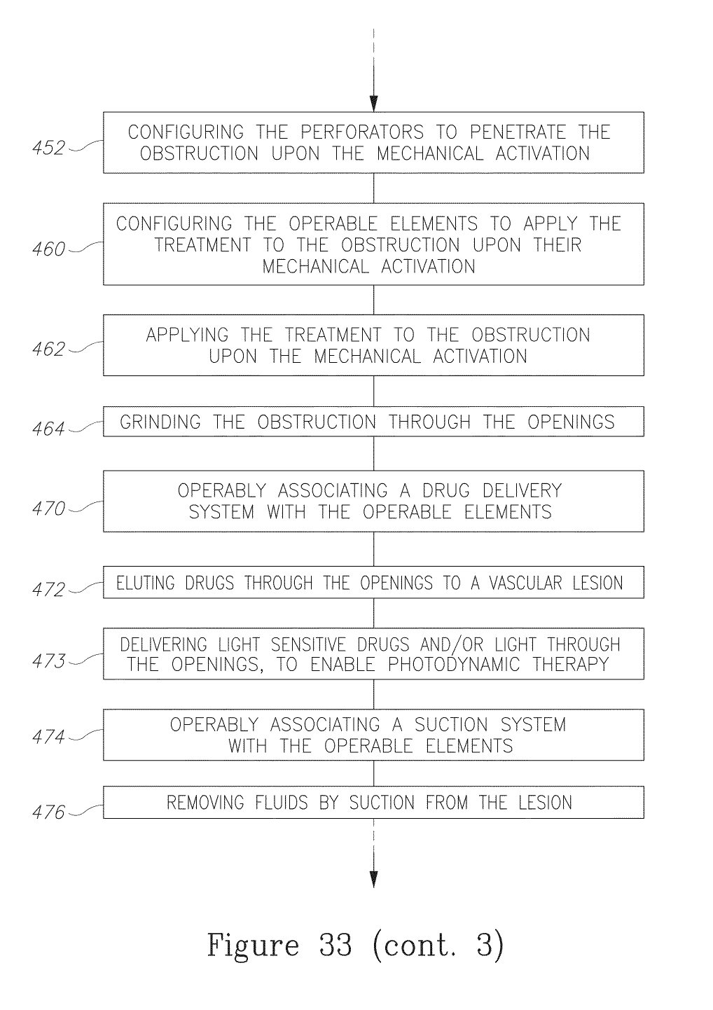

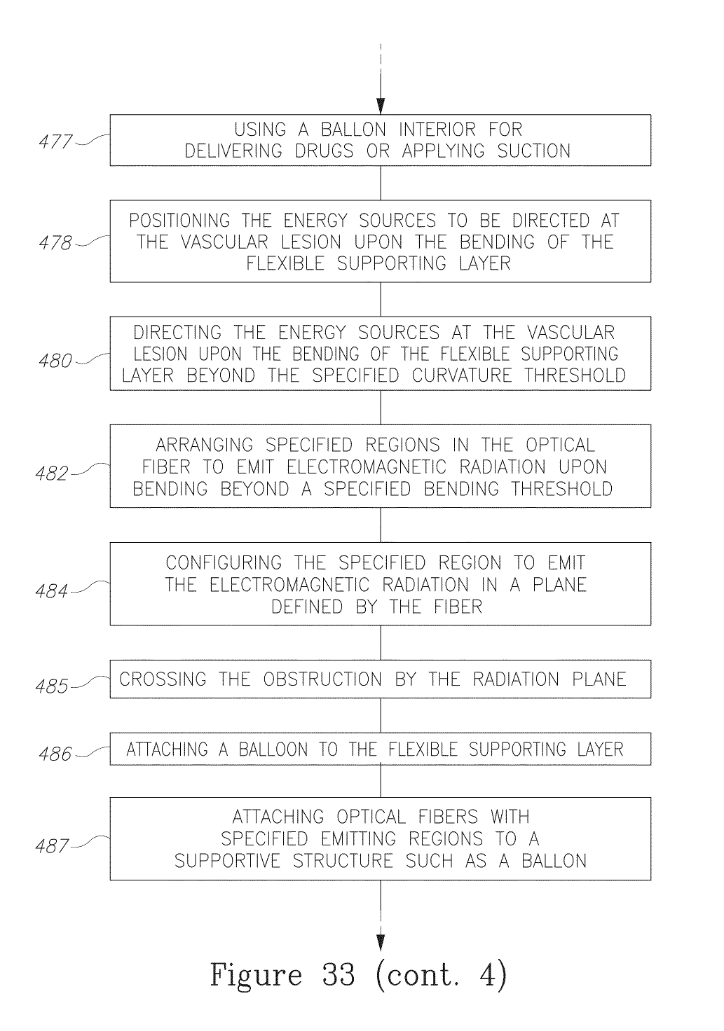

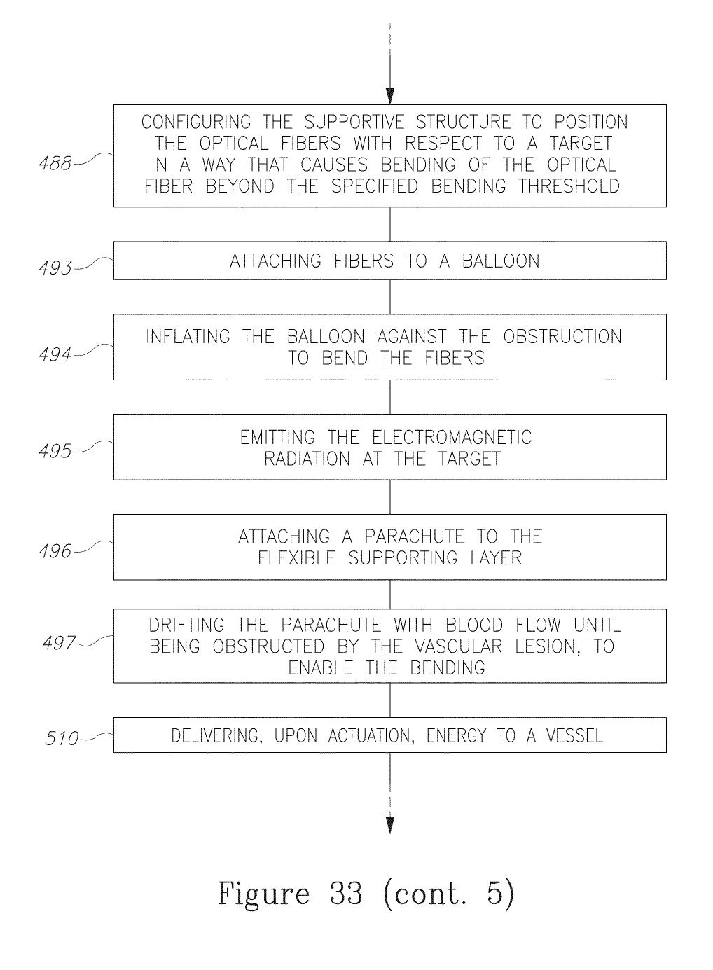

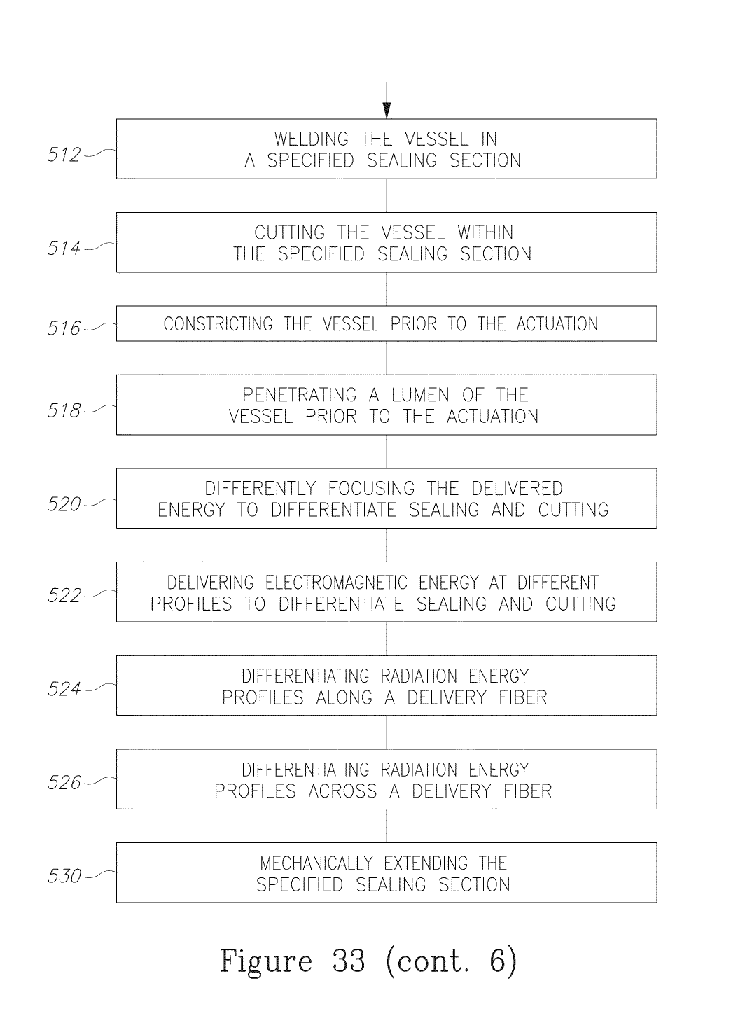

FIG. 33 is a high level flowchart illustrating a method 400, according to some embodiments of the invention. Method 400 comprises stages for treating an obstruction in a vessel, for configuring emission from an optical fiber and removing an obstruction therewith and for vessel sealing, according to some embodiments of the

Method 400 may comprise designing a device for applying a treatment to an obstruction (stage 410) comprises the following stages: attaching a treatment layer comprising a plurality of operable elements to a flexible supporting layer (stage 438); designing (stage 440) the operable elements to be mechanically activated (stage 442) upon bending of the flexible supporting layer beyond a specified curvature threshold (stage 444) due to contact of the treatment layer with the obstruction (such as a vascular lesion) (stage 446); and configuring the operable elements (stage 460) to apply the treatment (stage 462) to the obstruction upon their mechanical activation.

Method 400 may further comprise designing the operable elements to be mechanically de-activated upon a specified decrease of the bending (stage 443), due to the treatment of the obstruction.

Method 400 may further comprise designing the operable elements to comprise openings (stage 412) and configuring the openings to be opened mechanically by the mechanical activation (stage 448).

Method 400 may further comprise selecting an orientation of the operable elements with respect to a form of the obstruction (stage 449) for example to apply a spatially differentiated treatment. Curvature thresholds may be selected according to the orientation of the operable elements or vice versa. Curvature thresholds may vary for different orientations on obstruction 95.

Method 400 may further comprise covering the openings by corresponding discs (stage 414), and configuring the discs to expose the openings upon the mechanical activation (stage 450).

Method 400 may further comprise configuring the treatment layer as a flexible network in which mesh openings widen upon the bending (stage 415). The treatment may be applied utilizing the wider mesh openings to treat the lesion specifically.

Method 400 may further comprise designing the operable elements to comprise perforators (stage 416), and configuring the perforators to penetrate the obstruction (e.g. a vascular lesion) upon the mechanical activation (stage 452).

Method 400 may further comprise operably associating a drug delivery system with the operable elements (stage 470) and eluting drugs to the lesion thereby (stage 472). Method 400 may further comprise delivering light sensitive drugs and/or light through the openings, to enable photodynamic therapy (stage 473).

Method 400 may further comprise operably associating a suction system with the operable elements (stage 474) and removing fluids by suction from the lesion thereby (stage 476).

Method 400 may further comprise designing the operable elements to comprise energy sources (418), and positioning the energy sources (stage 478) to be directed (480) at the obstruction upon the bending of the flexible supporting layer beyond the specified curvature threshold (444).

Method 400 may further comprise attaching a balloon to the flexible supporting layer (stage 486) to enable the bending (stage 444) by inflating the balloon (stage 292).

Method 400 may further comprise attaching a parachute to the flexible supporting layer (stage 496) to enable the bending (stage 444) by drifting the parachute with flow (e.g. blood flow) until being obstructed by the vascular lesion (stage 497).

In embodiments, method 400 comprises treating an obstruction (stage 405) by mechanically activating operable elements of a bendable device (stage 442) by bending the device onto the obstruction (stage 444).

For example, treating a vascular lesion (stage 405) comprises eluting drugs into the lesion (stage 472) through openings, exposed (stage 448) or perforated (stage 452) by the operable elements. In another example, treating the vascular lesion (stage 205) comprises removing fluids by suction from the lesion (stage 476) through openings, exposed (stage 448) or perforated (stage 452) by the operable elements.

In embodiments, treating the vascular lesion (stage 405) comprises directing the energy sources at the vascular lesion upon the bending of the flexible supporting layer beyond the specified curvature threshold (stage 480).

Contacting the vascular lesion may be carried out by inflating a balloon attached (stage 486) to the flexible supporting layer or by drifting a parachute attached to the flexible supporting layer (stage 296) with blood flow until being obstructed by the vascular lesion, to enable the bending (stage 297).

Method 400 may further comprise configuring emission from an optical fiber (stage 421) and removing an obstruction therewith (stage 432), according to some embodiments of the invention.

Method 400 comprises arranging at least one specified region in the optical fiber to emit transferred electromagnetic radiation from a core through a cladding of the optical fiber upon bending of the optical fiber at the at least one specified region beyond a specified bending threshold (stage 482).

Method 400 may further comprise configuring the cladding to be asymmetric in cross section at the at least one specified region (stage 422) and to comprise at least one emission sector and at least one non-emitting sector. Method 400 may further comprise setting an effective refractive index n_{E} of the at least one emission sector closer to a core refractive index n_{K} than a cladding refractive index n_{M} (stage 423), to yield transmission through the at least one emission sector upon bending of the optical fiber at the at least one specified region beyond a specified bending threshold.

Setting the effective refractive index n_{E} (stage 423) may be carried out by at least one of: changing n_{M} to n_{E} (stage 424); serrating the cladding (stage 425); micro-structuring the cladding (stage 426); optionally positioning the core asymmetrically or eccentrically within the cladding (stage 427); applying an asymmetric coating onto the cladding (stage 428); and straining the fiber (stage 429), e.g., mechanically, thermally, or using radiation (e.g. ultraviolet light).

Method 400 may comprise configuring the at least one specified region to emit the electromagnetic radiation in a plane defined by the fiber (stage 484) and removing the obstruction (stage 432) by crossing the obstruction by the plane (stage 485).

Method 400 may further comprise attaching at least one optical fiber with the at least one specified region to a supportive structure (stage 487) and configuring the supportive structure to position the at least one optical fiber with respect to a target in a way that causes bending of the at least one optical fiber beyond the specified bending threshold thereof over the target (stage 488), to emit the electromagnetic radiation at the target (stage 495).

Removing the obstruction (stage 432) may be carried out by applying to the obstruction at least one specified region of at least one optical fiber arranged to emit transferred electromagnetic radiation from a core through a cladding of the optical fiber upon bending of the optical fiber at the at least one specified region beyond a specified bending threshold (stage 430).

The at least one optical fiber may be applied to the obstruction (stage 430) by inflating a balloon (stage 494) to which the at least one optical fiber is attached (stage 493) against the obstruction to yield bending of the at least one optical fiber at the at least one specified region beyond a specified bending threshold.

In embodiments, device 100 and method 400 are used to assist in medical procedures, specifically by soft tissue cutting utilizing fiber-optic bends for laser energy discharge. The natural curvature in clinical settings is used to induce safe, controlled and predetermined laser discharge from the fiber's side for various purposes like soft tissue, cartilage and bone cutting, ablation and coagulation. Furthermore the controlled discharge may be used for other purposes without limitation such as sensing, material processing and other applications.

Device 100 and method 400 may be used for cutting and/or removing polyps, lesions, soft tissue sarcomas, benign and malignant tumors and other soft tissue applications. In another embodiment it is used for moderate tissue damage treatments like atrial fibrillation, denervation or nerve inhibition and/or excitation and drug activation through photodynamic therapy. Device 100 and method 400 may be used to provide means of transporting of light energy to the desired location, adjusted to the specific geometry and emission of energy to the tissue to allow safe ablation/coagulation/cutting of the tissue in a precise and controlled manner. Device 100 and method 400 may be used in different parts of the animal or human body, such as: the colon, ileum, cecum, esophagus, stomach and all other parts of the digestive system; the urethra, urinary bladder, ureter, kidney or any other part of the urinary system also the vagina, cervix, uterus, ovaries, prostate gland, penis and all other parts of the reproductive system; the nasal and oral cavities, the epiglottis, trachea, bronchus, lungs and all other parts of the respiratory system; the brain, spinal cord and all other parts of the nervous system; as well as all parts of the circulatory system, veins, arteries the heart etc. It may also operate on parts of the skin or dermatology related procedures, plastic and general surgery procedures.

In embodiments, device 100 and method 400 may be used to treat atherosclerosis by laser atherectomy by delivering the laser energy specifically to the calcified lesion by using its topography to induce the tight bends in the fiber, which in turn causes the beam to “leak” out of the fiber and be transmitted to the lesion. Thus, device 100 and method 400 take advantage of the geometry of the lesion to target the discharge exclusively at the treatment site.

The following are some non-limiting examples for bending thresholds with respect to various obstructions. It should be noted, as illustrated below, that generally there are two dimensions related to the bending, or curvature radius. One is in the radius of vessel 90 (in a plane perpendicular to the vessel) and the other is related to the longitudinal way along vessel 90 (in a longitudinal cross section of vessel 90). As a result, fiber 110 may be asymmetric and have different dimensions in different directions, or be oriented in different ways along device 100 (and with respect to vessel 90). Bending or curvature thresholds may differ in different directions, as in the longitudinal axis the reference radius (that of vessel 90 without any obstruction 95) is very large, while in the cross sectional axis the reference radius (without obstruction 95) is much smaller. Hence, different emission parameters may be defined for these directions and may be used to apply coarser of finer treatments, possibly simultaneously in different regions or sequentially and complementary in one region of obstruction 95.

In non-limiting examples, the following may be typical parameters for the bending threshold in cases of different obstructions. In case of coronary lesions having a radius between 0.1 mm and 2 mm, the specified bending threshold may be about two times the lesion radius, or up to five times the lesion radius to achieve a more thorough removal of the lesion. Fiber 110 and device 100 may be adapted or selected according to the specific lesion that is to be treated therewith.

In case of vascular lesions having a radius between 1 mm and 10 mm, the specified bending threshold may be about two times the lesion radius, or up to five times the lesion radius to achieve a more thorough removal of the lesion. Fiber 110 and device 100 may be adapted or selected according to the specific lesion that is to be treated therewith.

In case of airway obstruction having a radius between 0.5 mm and 5 mm for small obstructions and having a radius between 5 mm and 20 mm for large obstructions, the specified bending threshold may be about two times the obstruction radius, or up to five times the obstruction radius to achieve a more thorough removal of the obstruction. Fiber 110 and device 100 may be adapted or selected according to the specific obstruction that is to be treated therewith.

In case of gastrointestinal obstructions having a radius between 10 mm and 100 mm, the specified bending threshold may be about two times the obstruction radius, or up to five times the obstruction radius to achieve a more thorough removal of the obstruction. Fiber 110 and device 100 may be adapted or selected according to the specific obstruction that is to be treated therewith.

In case of obstructions in water or sewage pipes, having a radius between 50 mm and 500 mm, the specified bending threshold may be about two times the obstruction radius, or up to five times the obstruction radius to achieve a more thorough removal of the obstruction. Fiber 110 and device 100 may be adapted or selected according to the specific obstruction that is to be treated therewith.

Device 100 may be part of an arterial catheter system which includes a flexible elongate member or catheter with an outer surface, a distal region adapted to enter an artery and a proximal region extending from a patient's vessel, permitting control outside the patient's body by a physician. At the distal region of the catheter is the atherosclerosis treating assembly which may include suction and/or drug administration surface and a balloon expansion unit.

Method 400 may further comprise vessel sealing by delivering, upon actuation, energy to a vessel (stage 510) to yield a vessel welding effect in a specified sealing section of the vessel and to cut the vessel within the specified sealing section. Method 400 may comprise welding the vessel in a specified sealing section (stage 512) and cutting the vessel within the specified sealing section (stage 514). In certain embodiments, the welding and the cutting may be carried out by a single actuation. The delivered energy may comprise at least one of optical, electrical and ultrasound energy

For example, the delivered energy may be electromagnetic radiation and method 400 may further comprise creating the welding and cutting by differently focusing the delivered electromagnetic radiation on the specified sealing section and on the cutting location, respectively, to differentiate sealing and cutting (stage 520).

In another example, the delivered energy may be electromagnetic radiation method 400 may further comprise using at least one optical fiber arranged to emit the electromagnetic radiation at at least two radiation profiles, one corresponding to welding 512 and another corresponding to cutting 514 the vessel. Generally, certain embodiments may comprise delivering electromagnetic energy at different profiles to differentiate sealing and cutting (stage 522). For example, radiation energy profiles may be differentiated along a delivery fiber (stage 524), across a delivery fiber (stage 526) or by a combination thereof and in respect to the positioning of the delivery fibers in jaws of a forceps tip arranged to perform method 400.

In certain embodiments, method 400 may further comprise constricting the vessel prior to the actuation (stage 516). The constriction may be arranged to yield more effective sealing and/or cutting by reducing the vessel diameter and increasing the usable spatial variability of energy delivery.

In certain embodiments, method 400 may further comprise penetrating a lumen of the vessel prior to the actuation (stage 518). Penetrating the vessel enables sealing the vessel from within and thereby applying the delivered energy efficiently and in a controllable manner to seal and cut the vessel.

In certain embodiments, method 400 may further comprise mechanically extending the specified sealing section (stage 530). The extending may be carried out prior, during or after sealing the vessel to broaden the sealing section to allow more effective cutting and healing of the cutting location.

In the above description, an embodiment is an example or implementation of the invention. The various appearances of “one embodiment”, “an embodiment”, “certain embodiments” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their used in the specific embodiment alone.

Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A device for applying a treatment to an obstruction, the device comprising a flexible treatment layer that comprises a plurality of operable elements configured to be activated to apply the treatment to the obstruction upon bending of the flexible treatment layer beyond a specified curvature threshold and to be de-activated upon a specified decrease of the bending.

2. The device of claim 1, wherein the operable elements are energy sources configured to deliver energy to the obstruction and positioned to be directed upon the bending of the flexible supporting layer beyond the specified curvature threshold at the obstruction.

3. The device of claim 1, configured as a balloon with the flexible supporting layer being part of or externally attached to a skin of the balloon, wherein the bending of the flexible supporting layer is carried out by inflating the balloon against the obstruction.

4. The device of claim 1, wherein the obstruction is a vascular lesion and the treatment layer is configured to treat the vascular lesion.

5. The device of claim 1, wherein the operable elements configured to be activated due to contact thereof with the obstruction and de-activated upon reduction of the bending resulting from the treatment of the obstruction.

6. The device of claim 1, wherein the treatment layer comprises at least one optical fiber comprising a core having a refractive index and a cladding having a refractive index, wherein the operable elements comprise at least one specified region of the fiber's cladding that is arranged to emit electromagnetic radiation from the core upon bending the optical fiber at the at least one specified region beyond a specified bending threshold.

7. The device of claim 6, wherein the cladding is asymmetric in cross section at the at least one specified region and comprises at least one emission sector and at least one non-emitting sector.

8. The device of claim 6, wherein the at least one emission sector comprises at least two emission sectors that differ in their specified bending threshold for emission.

9. The device of claim 6, wherein the cladding comprises, at the at least one specified region, at least one circle segment having an arc defining the at least one emission sector and further emitting the electromagnetic radiation at a plane.

10. The device of claim 9, wherein the at least one optical fiber is formed as a snare arranged to emit the electromagnetic radiation in a plane defined by the snare.

11. The device of claim 1, configured as a vessel sealing tip for surgical forceps, wherein the treatment layer comprises at least one optical fiber having the operable elements which are arranged to deliver, upon actuation, electromagnetic radiation to a vessel to yield a vessel welding effect in a specified sealing section of the vessel and to cut the vessel within the specified sealing section.

12. The device of claim 11, wherein at least one jaw of the forceps comprises at least one protrusion arranged to constrict the vessel prior to the actuation of the operable elements.

13. The device of claim 12, wherein the at least one protrusion is configured to contact tissue held by the tip and deliver both pressure and external energy to the tissue, wherein the pressure is a tip holding force concentrated by the at least one protrusion and the external energy delivered by the operable elements is at least one of optical, electrical and ultrasound energy

14. The device of claim 1, wherein the treatment layer comprises at least one optical fiber arranged to emit at least two radiation types at at least two corresponding zones of the fiber, the radiation types differing in at least one of: intensity, spectral range and temporal pattern.

15. A method of designing a device for applying a treatment to an obstruction, the method comprising:
attaching a treatment layer comprising a plurality of operable elements to a flexible supporting layer;
designing the operable elements to be activated upon bending of the flexible supporting layer beyond a specified curvature threshold; and
configuring the operable elements to apply the treatment to the obstruction upon their activation.

16. The method of claim 15, further comprising designing the operable elements to be activated due to contact thereof with the obstruction and de-activated upon reduction the bending resulting from the treatment of the obstruction.

17. The method of claim 15, further comprising integrating at least one optical fiber into the treatment layer and configuring the operable elements to comprise at least one specified region of a fiber's cladding that is arranged to emit electromagnetic radiation from a fiber's core upon bending the optical fiber at the at least one specified region beyond a specified bending threshold.

18. The method of claim 17, further comprising configuring the cladding to be asymmetric in cross section at the at least one specified region and to comprise at least one emission sector and at least one non-emitting sector, and setting an effective refractive index n_{E} of the at least one emission sector closer to a core refractive index n_{K} than a cladding refractive index n_{M}, to yield transmission through the at least one emission sector upon bending of the optical fiber at the at least one specified region beyond the specified bending threshold.

19. The method of claim 17, further comprising configuring the treatment layer as a vessel sealing tip for surgical forceps, and configuring the operable elements to deliver, upon actuation, electromagnetic radiation to a vessel to yield a vessel welding effect in a specified sealing section of the vessel and to cut the vessel within the specified sealing section.

20. A method of treating a vessel or a vessel obstruction by applying thereto energy from a plurality of operable elements within a treatment layer attached to a flexible supporting layer, wherein the operable elements are configured to be activated upon bending of the flexible supporting layer beyond a specified curvature threshold to apply the energy.
